(19) United States
(12) Patent Application Publication
(10) Pub. No.: US 2021/0137886 A1
(43) Pub. Date: May 13, 2021

(54) INHIBITORS OF METALLO-BETA-LACTAMASES

(71) Applicant: Oxford University Innovation Limited, Oxford (GB)

(72) Inventors: Johan GISING, Oxford (Oxfordshire) (GB); Stefan LINDSTROM, Oxford (Oxfordshire) (GB); Dmitry ANTONOV, Oxford (Oxfordshire) (GB); Peter BRANDT, Oxford (Oxfordshire) (GB); Anna Karin BELFRAGE, Oxford (Oxfordshire) (GB); Jürgen BREM, Oxford (Oxfordshire) (GB); Christopher J. SCHOFIELD, Oxford (Oxfordshire) (GB)

(21) Appl. No.: 16/616,513

(22) PCT Filed: May 25, 2018
(86) PCT No.: PCT/GB2018/051446

§ 371 Date: Nov. 25, 2019

(30) Foreign Application Priority Data
May 26, 2017 (GB) 1708451.8

(51) Int. Cl. A61K 31/407; A61P 31/04; C07D 495/04; C07D 487/04; A61K 31/41; A61K 31/4439; A61K 31/454; A61K 31/4245; A61K 31/4162

(52) U.S. Cl. CPC A61K 31/407; A61P 31/04; C07D 495/04; C07D 487/04; A61K 31/4162; A61K 31/4439; A61K 31/454; A61K 31/4245; A61K 31/41

(57) ABSTRACT

The present invention relates to certain compounds that function as inhibitors of bacterial metallo-beta-lactamases. The present invention also relates to processes for the preparation of these compounds, to pharmaceutical compositions comprising them, and to their use in the treatment of a bacterial infection.

### INTRODUCTION

The present invention relates to compounds that function as inhibitors of metallo-beta-lactamases. The present invention also relates to processes for the preparation of these compounds, to pharmaceutical compositions comprising them, and to their use in the treatment of bacterial infections.

### BACKGROUND OF THE INVENTION

Infections caused by pathogenic bacteria are common worldwide, and thus antibacterial medicines to treat such infections are highly sought. Currently, β-lactam antibacterials (BLAs) are amongst the most widely used antibacterial treatments.¹ However, the efficacy of BLAs is increasingly threatened by bacterial resistance, most importantly by the widespread dissemination of β-lactamases, which catalyse the hydrolysis and inactivation of BLA.²

In combination with a suitable penicillin, Class A β-lactamase inhibitors (BLIs) have been components of highly successful medicines (e.g. as in Augmentin). However, the zinc ion dependent Class B metallo-β-lactamases (MBLs, or carbapenemases), are structurally and mechanistically distinct from Class A, C and D serine β-lactamases (SBLs).³ There is therefore a need for effective inhibitors of MBLs.

MBLs are particularly concerning because they hydrolyse most known BLAs, including the so called ‘last resort’ BLAs, such as some carbapenems, and confer resistance to BLAs in many pathogens. No clinically useful MBL inhibitors (MBLIs) are presently available.⁴

Though the problem of BLA resistance is most pronounced in developing countries, the number of cases of antimicrobial resistance (AMR) including Carbapenem-resistant Enterobacteriaceae (CRE) is substantially increasing worldwide.⁵ It is notable that the estimates in these reports may under-represent the actual problem of BLA resistance, due to a lack of broad surveillance programs in some countries (many countries have not allocated, or do not have the resources for surveillance programs). A recent report shows NDM-1 is the most relevant MBL in the United Kingdom.⁶ Similar reports are also appearing worldwide.

Thus, there remains a need for new treatments to combat MBL mediated antibacterial resistance.

The present invention was devised with the foregoing in mind.

### SUMMARY OF THE INVENTION

In one aspect, the present invention provides a compound as defined herein, or a pharmaceutically acceptable salt or solvate thereof.

In another aspect, the present invention provides a compound as defined herein, or a pharmaceutically acceptable salt or solvate thereof, for use in the treatment of a bacterial infection.

In another aspect, the present invention provides a compound as defined herein, or a pharmaceutically acceptable salt or solvate thereof, in combination with a suitable antibacterial agent, for use in the treatment of a bacterial infection.

In another aspect, the present invention provides a pharmaceutical composition as defined herein which comprises a compound as defined herein, or a pharmaceutically acceptable salt or solvate thereof, and one or more pharmaceutically acceptable excipients.

In another aspect, the present invention provides a compound as defined herein, or a pharmaceutically acceptable salt or solvate thereof, or a pharmaceutical composition as defined herein, for use in the treatment of bacterial infections.

In another aspect, the present invention provides a compound as defined herein, or a pharmaceutically acceptable salt or solvate thereof, or a pharmaceutical composition as defined herein, for use in the production of a metallo-beta-lactamase inhibitory effect.

In another aspect, the present invention provides a method of inhibiting a bacterial metallo-beta-lactamase in vitro or in vivo, said method comprising contacting a cell with an effective amount of a compound as defined herein, or a pharmaceutically acceptable salt or solvate thereof.

In another aspect, the present invention provides a method of treating a bacterial infection in a patient or animal in need of such treatment, said method comprising administering to said patient a therapeutically effective amount of a compound as defined herein, or a pharmaceutically acceptable salt or solvate thereof, or a pharmaceutical composition as defined herein, in combination with a suitable antibacterial agent.

In another aspect, the present invention provides the use of a compound, as defined herein, in combination with a suitable antibacterial agent, for the treatment of a bacterial infection.

In another aspect, the present invention provides the use of a compound, as defined herein, for the inhibition of a metallo-beta-lactamase

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups and analogues thereof. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkylene” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). The term heterocyclyl includes both monovalent species and divalent species. Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4th Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:

a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzofuranyl, benzothiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In one aspect, the present invention relates to a compound of formula I or II, or a pharmaceutically acceptable salt or solvate thereof, as shown below:

[chemical structure]

wherein

A₁ is selected from C, N, O, S, S(O) or S(O)₂;

A₂ is selected from C, N, O, S, S(O) or S(O)₂;

A₃ is selected from C, N, O, S, S(O) or S(O)₂;

with the proviso that either:
(i) all of A₁, A₂ and A₃ are C;
(ii) one of A₁, A₂ and A₃ is selected from N, O, S, S(O) or S(O)₂ and the others are C; or
(iii) two of A₁, A₂ and A₃ are N and the other is C;

R₁ is selected from hydrogen, (1-4C)alkyl, (1-4C)alkylaryl or aryl, wherein each (1-4C)alkyl or aryl is optionally substituted by one or more substituent groups selected from oxo, halo, cyano, nitro, hydroxy, carboxy, NR^{1A}R^{1B} or (1-4C)alkoxy, wherein R^{1A} and R^{1B} are each independently selected from hydrogen or (1-2C)alkyl;

R₂ is selected from hydrogen or:
(i) —C(O)OH;
(ii) —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
(iii) —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
(iv) —C(O)NR_{2D}NR_{2B}R_{2E}; wherein R_{2D} is selected from hydrogen or (1-6C)alkyl and R_{2B} and R_{2C} are as defined above;
(v) tetrazolyl;
(vi) triazolyl;
(vii) —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
(viii) trifluoromethylketone;
(ix) cyano;
(x) —[C(O)]ᵥS(NR_{2X})(O)NR_{2B}R_{2C} (where v is 0 or 1), wherein R_{2X} is selected from hydrogen, (1-6C)alkyl, C(O)R_{X}, C(O)OR_{X}, aryl, aryl-(1-2C)alkyl, heteroaryl, heteroaryl-(1-2C)alkyl, heterocyclyl or heterocyclyl-(1-2C)alkyl, wherein R_{X} is selected from (1-6C)alkyl, aryl or heteroaryl, each of which is optionally substituted with one or more R_{A}, and wherein R_{2B} and R_{2C} are as defined above;
(xi) —[C(O)]_{w}N(R_{2B})S(NR_{2X})(O)R_{X} (where w is 0 or 1), wherein R_{2B}, R_{2X} and R_{X} are each as defined above; or
(xii) —S(O)_{y}NR_{2B}R_{2C} (wherein y is 1 or 2), and wherein R_{2B} and R_{2C} are as defined above,

and wherein R^{A} is selected from oxo, halo, cyano, nitro or a group of the formula:

—Y²—X²—Z²

wherein
Y² is absent or a linker group of the formula —[CR^{A1}R^{A2}]ₘ— in which m is an integer selected from 1, 2, 3 or 4, and R^{A1} and R^{A2} are each independently selected from hydrogen or (1-2C)alkyl;
X² is absent or —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —S—, —SO—, —SO₂—, —S(O)₂N(R^{A3})—, or —N(R^{A3})SO₂— wherein R^{A3} is selected from hydrogen or methyl; and
Z² is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z² is optionally further substituted by one or more substituent groups independently selected from oxo, halo, cyano, nitro, hydroxy, carboxy, NR^{A4}R^{A5}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, heterocyclyl, heterocyclyloxy, heterocyclyl-(1-2C)alkyl, heteroaryl, heteroaryloxy, heteroaryl-(1-2C)alkyl, C(O)NR^{A4}R^{A5}, NR^{A4}C(O)R^{A5}, NR^{A4}S(O)₂R^{A5} and S(O)₂NR^{A4}R^{A5}; wherein R^{A4} and R^{A5} are each independently selected from hydrogen, (1-4C)alkyl or (3-6C)cycloalkyl or (3-6C)cycloalkyl(1-2C)alkyl; or R^{A4} and R^{A5} can be linked such that, together with the nitrogen atom to which they are attached, they form a 4-6 membered heterocyclic ring;
   and wherein any alkyl, aryl, heterocyclyl or heteroaryl group present in a substituent group on Z² is optionally further substituted by halo, cyano, nitro, hydroxy, caboxy, NR^{A6}R^{A7}, (1-2C)alkoxy, or (1-2C)alkyl; wherein R^{A6} and R^{A7} are selected from hydrogen or (1-2C)alkyl;

R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};

R^{B} is oxo, halo, cyano, nitro, hydroxy or a group:

—Y³—X³—Z³

wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
   and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl;
or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
   m is 0 or 1;

R₄ is selected from halo, cyano, nitro, hydroxy or a group

—Y⁴—X⁴—Z⁴

wherein
Y⁴ is absent or a linker group of the formula —[CR^{4A}R^{4B}]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, —N(R^{4D})SO₂— or ═N—O— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring;
   and wherein any alkyl, aryl, heterocyclyl or heteroaryl group present in a substituent group on Z⁴ is optionally further substituted by oxo, halo, cyano, nitro, hydroxy, carboxy, NR^{4G}R^{4H}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4G}, OC(O)R^{4H}, C(O)NR^{4G}R^{4H}, NR^{4G}C(O)R^{4H}, NR^{4G}S(O)₂R^{4H} and S(O)₂NR^{4G}R^{4H}; wherein R^{4G} and R^{4H} are each independently selected from hydrogen, (1-4C)alkyl or (3-6C)cycloalkyl or (3-6C)cycloalkyl(1-2C)alkyl; or R^{4G} and R^{4H} can be linked such that, together with the nitrogen atom to which they are attached, they form a 4-7 membered heterocyclic ring;
or R^{4C} and Z⁴ can be linked such that, together with the nitrogen atom to which they are attached, they form a 4-7 membered heterocyclic ring, which is optionally substituted by oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, or C(O)NR^{4E}R^{4F}, NR^{4E}C(O)R^{4F}, NR^{4E}S(O)₂R^{4F} and S(O)₂NR^{4E}R^{4F};
   n is 0 or 1 and R₅ is selected from halo, cyano, nitro, hydroxy or a group

—Y⁵—X⁵—Z⁵

wherein:
Y⁵ is absent or a linker group of the formula —[CR^{5A}R^{5B}]_{q}— in which q is an integer selected from 1 or 2 and R^{5A} and R^{5B} are each independently selected from hydrogen or methyl;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, nitro, hydroxy, carboxy, NR^{5E}R^{5F} or (1-2C)alkoxy; wherein R^{5E} and R^{5F} are each independently selected from hydrogen or (1-2C)alkyl; or
   n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, nitro, hydroxy or a group

—Y⁶—X⁶—Z⁶

wherein:
Y⁶ is absent or a linker group of the formula —[CR^{6A}R^{6B}]_{q}— in which q is an integer selected from 1 or 2 and R^{6A} and R^{6B} are each independently selected from hydrogen or methyl;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or (1-4C)alkyl.

In another aspect, the present invention provides a compound of formula I, or a pharmaceutically acceptable salt or solvate thereof, of Formula I:

[chemical structure]

wherein

A₁ is selected from C, N, O, S, SO or S(O)₂;

A₂ is selected from C, N, O, S, SO or S(O)₂;

A₃ is selected from C, N, O, S, SO or S(O)₂;

with the proviso that either:
(i) all of A₁, A₂ and A₃ are C;
(ii) one of A₁, A₂ and A₃ is selected from N, O, S or S(O)₂ and the others are C; or
(iii) two of A₁, A₂ and A₃ are N and the other is C; R₁ is selected from hydrogen, (1-4C)alkyl or aryl, wherein each (1-4C)alkyl or aryl is optionally substituted by one or more substituent groups selected from oxo, halo, cyano, nitro, hydroxy, carboxy, NR^{1A}R^{1B} or (1-4C)alkoxy, wherein R^{1A} and R^{1B} are each independently selected from hydrogen or (1-2C)alkyl;

R₂ is selected from:
(i) —C(O)OH;
(ii) —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
(iii) —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
(iv) —C(O)NR_{2D}NR_{2B}R_{2E}; wherein R_{2D} is selected from hydrogen or (1-6C)alkyl and R_{2B} and R_{2C} are as defined above;
(v) tetrazolyl;
(vi) triazolyl;
(vii) —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
(viii) trifluoromethylketone;
(ix) cyano;
(x) —[C(O)]ᵥS(NR_{2X})(O)NR_{2B}R_{2C} (where v is 0 or 1), wherein R_{2X} is selected from hydrogen, (1-6C)alkyl, C(O)R_{X}, C(O)OR_{X}, aryl, aryl-(1-2C)alkyl, heteroaryl, heteroaryl-(1-2C)alkyl, heterocyclyl or heterocyclyl-(1-2C)alkyl, wherein R_{X} is selected from (1-6C)alkyl, aryl or heteroaryl, each of which is optionally substituted with one or more R_{A}, and wherein R_{2B} and R_{2C} are as defined above;
(xi) —[C(O)]_{w}N(R_{2B})S(NR_{2X})(O)R_{X} (where w is 0 or 1), wherein R_{2B}, R_{2X} and R_{X} are each as defined above; or
(xii) —S(O)_{y}NR_{2B}R_{2C} (wherein y is 1 or 2), and wherein R_{2B} and R_{2C} are as defined above,
and wherein R^{A} is selected from oxo, halo, cyano, nitro or a group of the formula:

—Y²—X²—Z²

wherein
Y² is absent or a linker group of the formula —[CR^{A1}R^{A2}]ₘ— in which m is an integer selected from 1, 2, 3 or 4, and R^{A1} and R^{A2} are each independently selected from hydrogen or (1-2C)alkyl;
X² is absent or —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —S—, —SO—, —SO₂—, —S(O)₂N(R^{A3})—, or —N(R^{A3})SO₂— wherein R^{A3} is selected from hydrogen or methyl; and
Z² is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z² is optionally further substituted by one or more substituent groups independently selected from oxo, halo, cyano, nitro, hydroxy, carboxy, NR^{A4}R^{A5}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, heterocyclyl, heterocyclyloxy, heterocyclyl-(1-2C)alkyl, heteroaryl, heteroaryloxy, heteroaryl-(1-2C)alkyl, C(O)NR^{A4}R^{A5}, NR^{A4}C(O)R^{A5}, NR^{A4}S(O)₂R^{A5} and S(O)₂NR^{A4}R^{A5}; wherein R^{A4} and R^{A5} are each independently selected from hydrogen, (1-4C)alkyl or (3-6C)cycloalkyl or (3-6C)cycloalkyl(1-2C)alkyl; or R^{A4} and R^{A5} can be linked such that, together with the nitrogen atom to which they are attached, they form a 4-6 membered heterocyclic ring;
   and wherein any alkyl, aryl, heterocyclyl or heteroaryl group present in a substituent group on Z² is optionally further substituted by halo, cyano, nitro, hydroxy, caboxy, NR^{A6}R^{A7}, (1-2C)alkoxy, or (1-2C)alkyl; wherein R^{A6} and R^{A7} are selected from hydrogen or (1-2C)alkyl;

R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};

R^{B} is halo, cyano, nitro, hydroxy or a group:

—Y³—X³—Z³

wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
   and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl;
or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
   m is 0 or 1;

R₄ is selected from halo, cyano, nitro, hydroxy or a group

—Y⁴—X⁴—Z⁴

wherein
Y⁴ is absent or a linker group of the formula —[CR^{4A}R^{4B}]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring;
   and wherein any alkyl, aryl, heterocyclyl or heteroaryl group present in a substituent group on Z⁴ is optionally further substituted by halo, cyano, nitro, hydroxy, caboxy, NR^{4G}R^{4H}, (1-2C)alkoxy, or (1-2C)alkyl; wherein R^{4G} and R^{4H} are selected from hydrogen or (1-2C)alkyl;
or R^{4C} and Z⁴ can be linked such that, together with the nitrogen atom to which they are attached, they form a 4-7 membered heterocyclic ring, which is optionally substituted by oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, or C(O)NR^{4E}R^{4F}, NR^{4E}C(O)R^{4F}, NR^{4E}S(O)₂R^{4F} and S(O)₂NR_{4E}R^{4F};
   n is 0 or 1 and R₅ is selected from halo, cyano, nitro, hydroxy or a group

—Y⁵—X⁵—Z⁵

wherein:
Y⁵ is absent or a linker group of the formula —[CR^{5A}R^{5B}]_{q}— in which q is an integer selected from 1 or 2 and R^{5A} and R^{5B} are each independently selected from hydrogen or methyl;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
   Z⁵ is hydrogen or (1-4C)alkyl which is optionally further substituted by one or more substituent groups independently selected from oxo, halo, cyano, nitro, hydroxy, carboxy, NR^{5E}R^{5F} or (1-2C)alkoxy; wherein R^{5E} and R^{5F} are each independently selected from hydrogen or (1-2C)alkyl; or
      n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, nitro, hydroxy or a group

—Y⁶—X⁶—Z⁶

wherein:
Y⁶ is absent or a linker group of the formula —[CR^{6A}R^{6B}]_{q}— in which q is an integer selected from 1 or 2 and R^{6A} and R^{6B} are each independently selected from hydrogen or methyl;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or (1-4C)alkyl.

Particular compounds of the invention include, for example, compounds of the formula I or II, or pharmaceutically acceptable salts and/or solvates thereof, wherein, unless otherwise stated, each of A₁, A₂, A₃, R₁, R₂, R₃, R₄ and R₅ and any associated substituent groups has any of the meanings defined hereinbefore or in any of paragraphs (1) to (63) hereinafter:—
(1) A₁ is selected from C, N, O or S;
(2) A₁ is selected from C, N or O;
(3) A₁ is selected from C or N;
(4) A₁ is C;
(5) A₁ is N;
(6) A₁ is O;
(7) A₁ is S;
(8) A₁ is S(O)₂;
(9) A₂ is selected from C, N, O or S;
(10) A₂ is selected from C, N or O;
(11) A₂ is selected from C or N;
(12) A₂ is C;
(13) A₂ is N;
(14) A₂ is O;
(15) A₂ is S;
(16) A₂ is S(O)₂;
(17) A₃ is selected from C, N, S or S(O)₂;
(18) A₃ is selected from C, N, O or S;
(19) A₃ is selected from C or N;
(20) A₃ is C;
(21) A₃ is N;
(22) A₃ is O;
(23) A₃ is S;
(24) A₃ is S(O)₂;
(25) all of A₁, A₂ and A₃ are C or one of A₁, A₂ and A₃ is selected from N, O, S or S(O)₂ and the others are C; or two of A₁, A₂ and A₃ are N and the other is C;
(26) all of A₁, A₂ and A₃ are C or one of A₁, A₂ and A₃ is selected from N, O, S or S(O)₂ and the others are C;
(27) R₁ is selected from hydrogen or (1-4C)alkyl, wherein each (1-4C)alkyl is optionally substituted by one or more substituent groups selected from oxo, halo, cyano, nitro, hydroxy, carboxy, NR^{1A}R^{1B} or (1-4C)alkoxy, wherein R^{1A} and R^{1B} are each independently selected from hydrogen or (1-2C)alkyl;
(28) R₁ is selected from hydrogen or (1-4C)alkyl which is optionally substituted by one or more substituent groups selected from oxo, halo, cyano, carboxy, NR^{1A}R^{1B} or (1-4C)alkoxy, wherein R^{1A} and R^{1B} are each independently selected from hydrogen or (1-2C)alkyl;
(29) R₁ is selected from hydrogen, (1-4C)alkyl or phenyl, wherein each (1-4C)alkyl or phenyl is optionally substituted by one or more substituent groups selected from oxo, halo, cyano, NR^{1A}R^{1B} or (1-2C)alkoxy, wherein R^{1A} and R^{1B} are each independently selected from hydrogen or methyl;
(30) R₁ is selected from hydrogen or (1-4C)alkyl which is optionally substituted by one or more substituent groups selected from oxo, halo, cyano, NR^{1A}R^{1B} or (1-2C)alkoxy, wherein R^{1A} and R^{1B} are each independently selected from hydrogen or methyl;
(31) R₁ is selected from hydrogen, (1-4C)alkyl or phenyl, wherein each (1-4C)alkyl or phenyl is optionally substituted by one or more substituent groups selected from oxo, halo, or (1-2C)alkoxy;
(32) R₁ is selected from hydrogen or (1-4C)alkyl which is optionally substituted by one or more substituent groups selected from oxo, halo, or (1-2C)alkoxy;
(33) R₁ is selected from hydrogen or (1-4C)alkyl which is optionally substituted by one or more substituent groups selected from oxo, fluoro or chloro;
(34) R₁ is selected from hydrogen or (1-4C)alkyl which is optionally substituted by one or more fluoro groups;
(35) R₁ is selected from hydrogen or (1-2C)alkyl;
(36) R₁ is hydrogen;
(37) R₂ is selected from hydrogen or:
   (i) —C(O)OH;
   (ii) —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   (iii) —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   (iv) —C(O)NR_{2D}NR_{2B}R_{2C}; wherein R_{2D} is selected from hydrogen or (1-6C)alkyl and R_{2B} and R_{2C} are as defined above;
   (v) tetrazolyl;
   (vi) triazolyl;
   (vii) —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
   (viii) trifluoromethylketone;
   (ix) cyano;
   (x) —[C(O)]ᵥS(NR_{2X})(O)NR_{2B}R_{2C} (where v is 0 or 1), wherein R_{2X} is selected from hydrogen, (1-6C)alkyl, C(O)R_{X}, C(O)OR_{X}, aryl, aryl-(1-2C)alkyl, heteroaryl, heteroaryl-(1-2C)alkyl, heterocyclyl or heterocyclyl-(1-2C)alkyl, wherein R_{X} is selected from (1-6C)alkyl, aryl or heteroaryl, each of which is optionally substituted with one or more R_{A}, and wherein R_{2B} and R_{2C} are as defined above;
   (xi) —[C(O)]_{w}N(R_{2B})S(NR_{2X})(O)R_{X} (where w is 0 or 1), wherein R_{2B}, R_{2X} and R_{X} are each as defined above; or
   (xii) —S(O)_{y}NR_{2B}R_{2C} (wherein y is 1 or 2), and wherein R_{2B} and R_{2C} are as defined above,
   and wherein R^{A} is selected from halo, cyano, nitro or a group of the formula:

—Y²—X²—Z²
wherein
Y² is absent or a linker group of the formula —[CR^{A1}R^{A2}]ₘ— in which m is an integer selected from 1 or 2, and R^{A1} and R^{A2} are each independently selected from hydrogen or (1-2C)alkyl;
X² is absent or —O—, —C(O)—, —C(O)O—, —OC(O)—, —N(R^{A3})—, —N(R^{A3})—C(O)—, —N(R^{A3})—C(O)O—, —C(O)—N(R^{A3})—, —N(R^{A3})C(O)N(R^{A3})—, —S(O)₂N(R^{A3})—, or —N(R^{A3})SO₂— wherein R^{A3} is selected from hydrogen or methyl; and
Z² is hydrogen, (1-6C)alkyl, aryl, (3-6C)cycloalkyl, (3-6C)cycloalkenyl, heteroaryl or heterocyclyl;
and wherein Z² is optionally further substituted by one or more substituent groups independently selected from oxo, halo, cyano, nitro, hydroxy, carboxy, NR^{A4}R^{A5}, (1-4C)alkoxy, (1-4C)alkyl, (3-8C)cycloalkyl, (1-4C)alkanoyl, (1-4C)alkylsulphonyl, C(O)NR^{A4}R^{A5}, NR^{A4}C(O)R^{A5}, NR^{A4}S(O)₂R^{A5} and S(O)₂NR^{A4}R^{A5}; wherein R^{A4} and R^{A5} are each independently selected from hydrogen, or (1-4C)alkyl; or R^{A4} and R^{A5} can be linked such that, together with the nitrogen atom to which they are attached, they form a 4-6 membered heterocyclic ring;
(38) R₂ is selected from hydrogen or:
   (i) —C(O)OH;
   (ii) —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   (iii) —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   (iv) —C(O)NR_{2D}NR_{2B}R_{2C}; wherein R_{2D} is selected from hydrogen or (1-6C)alkyl and R_{2B} and R_{2C} are as defined above;
   (v) tetrazolyl;
   (vi) triazolyl;
   (vii) —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
   (viii) trifluoromethylketone;
   (ix) cyano;
   (x) —[C(O)]ᵥS(NR_{2X})(O)NR_{2B}R_{2C} (where v is 0 or 1), wherein R_{2X} is selected from hydrogen or (1-6C)alkyl, and wherein R_{2B} and R_{2C} are as defined above;
   (xi) —[C(O)]_{w}N(R_{2B})S(NR_{2X})(O)R_{X} (where w is 0 or 1), wherein R_{2B}, R_{2X} and R_{X} are each as defined above; or
   (xii) —S(O)NR_{2B}R_{2C}, and wherein R_{2B} and R_{2C} are as defined above, and wherein R^{A} is selected from halo, cyano, nitro or a group of the formula:

—X²—Z²
wherein
X² is absent or —C(O)—, —N(R^{A3})—C(O)—, —C(O)—N(R^{A3})—, —SO₂—, wherein R^{A3} is selected from hydrogen or methyl; and
Z² is hydrogen, (1-6C)alkyl, aryl, (3-6C)cycloalkyl, heteroaryl or heterocyclyl;
and wherein Z² is optionally further substituted by one or more substituent groups independently selected from oxo, halo, cyano, hydroxy, NR^{A4}R^{A5}, (1-4C)alkoxy or (1-4C)alkyl, wherein R^{A4} and R^{A5} are each independently selected from hydrogen, or (1-2C)alkyl;
(39) R₂ is selected from hydrogen or:
   (i) —C(O)OH;
   (ii) —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   (iii) —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
   (iv) —C(O)NR_{2D}NR_{2B}R_{2C}; wherein R_{2D} is selected from hydrogen or (1-6C)alkyl and R_{2B} and R_{2C} are as defined above;
   (v) tetrazolyl;
   (vi)triazolyl;
   (vii) —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
   (viii) trifluoromethylketone;
(40) R₂ is selected from:
   (i) —C(O)OH;
   (ii) —C(O)NR_{2B}R_{2C}; wherein R_{2B} and R_{2C} are each independently selected from hydrogen, (1-6C)alkyl, (3-8C)cycloalkyl, aryl, heteroaryl or heterocyclyl, each of which is optionally substituted by one or more substituent groups R^{A};
   (iii) —C(O)NR_{2D}NR_{2B}R_{2C}; wherein R_{2D} is selected from hydrogen or (1-2C)alkyl and R_{2B} and R_{2C} are as defined above;
   (iv) tetrazolyl;
   (v) —B(OR_{2F})(OR_{2G}), wherein R_{2F} and R_{2G} are each independently selected from hydrogen, (1-4C)alkyl or R_{2F} and R_{2G} are linked such that, together with the B and O atoms, they form a 5 or 6-membered heterocyclic ring, which is optionally substituted by (1-2C)alkyl;
   and wherein R^{A} is selected from halo, cyano, or a group of the formula:

—X²—Z²
wherein
X² is absent or —C(O)—, —SO₂—; and
Z² is hydrogen, (1-6C)alkyl, aryl, or heteroaryl;
and wherein Z² is optionally further substituted by one or more substituent groups independently selected from halo, hydroxy, NR^{A4}R^{A5}, (1-4C)alkoxy or (1-4C)alkyl, wherein R^{A4} and R^{A5} are each independently selected from hydrogen, or (1-2C)alkyl;
(41) R₂ is selected from:
   (i) —C(O)OH;
   (ii) —C(O)NR_{2B}R_{2C}; wherein R_{2B} and R_{2C} are each independently selected from hydrogen, (1-6C)alkyl, (3-8C)cycloalkyl, aryl or heteroaryl, each of which is optionally substituted by one or more substituent groups R^{A};
   (iii) —C(O)NR_{2D}NR_{2B}R_{2C}; wherein R_{2D} is selected from hydrogen or (1-2C)alkyl and R_{2B} and R_{2C} are as defined above;
   (iv) tetrazolyl;
   (v) —B(OR_{2F})(OR_{2G}), wherein R_{2F} and R_{2G} are each independently selected from hydrogen, (1-4C)alkyl or R_{2F} and R_{2G} are linked such that, together with the B and O atoms, they form a 5 or 6-membered heterocyclic ring, which is optionally substituted by (1-2C)alkyl;
   and wherein R^{A} is selected from halo, cyano, or a group of the formula:

—X²—Z²
wherein
X² is absent or —C(O)—, —SO₂—; and
Z² is hydrogen, (1-6C)alkyl, aryl, or heteroaryl;
and wherein Z² is optionally further substituted by one or more substituent groups independently selected from halo, hydroxy, NR^{A4}R^{A5}, (1-4C)alkoxy or (1-4C)alkyl, wherein R^{A4} and R^{A5} are each independently selected from hydrogen, or (1-2C)alkyl;
(42) R₂ is selected from:
   (i) —C(O)OH;
   (ii) —C(O)NR_{2B}R_{2C}; wherein R_{2B} and R_{2C} are each independently selected from hydrogen, (1-6C)alkyl, aryl or heteroaryl, each of which is optionally substituted by one or more substituent groups R^{A};
   (iii)tetrazolyl;
   and wherein R^{A} is selected from halo, cyano, or a group of the formula:

—X²—Z²
wherein
X² is absent or —C(O)—, —SO₂—; and
Z² is hydrogen, (1-4C)alkyl, phenyl, or a 5- or 6-membered heteroaryl;
and wherein Z² is optionally further substituted by one or more substituent groups independently selected from halo, hydroxyl or (1-4C)alkyl;
(43) R₂ is selected from:
   (i) —C(O)OH;
   (ii) —C(O)NR_{2B}R_{2C}; wherein R_{2B} and R_{2C} are each independently selected from hydrogen, (1-6C)alkyl, aryl or heteroaryl, each of which is optionally substituted by one or more substituent groups R^{A};
   (iii) tetrazolyl;
   and wherein R^{A} is selected from halo, cyano or SO₂CH₃;
(44) R₂ is —C(O)OH or tetrazolyl;
(45) R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
   R^{B} is oxo, halo, cyano, nitro, hydroxy or a group:

—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
   and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl;
(46) R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
   R^{B} is oxo, halo, cyano, nitro, hydroxy or a group:

—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
(47) R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
   R^{B} is oxo, halo, cyano, nitro, hydroxy or a group:

—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
   and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
(48) R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
   R^{B} is oxo, halo, cyano, nitro, hydroxy or a group:

—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalky (1-2C)alkyl;
(49) R₃ is selected from hydrogen, halo, cyano, hydroxyl, aryl, (1-6C)alkyl, (2-6C)alkenyl, heteroaryl or heterocyclyl, wherein said aryl, heteroaryl or heterocyclyl is optionally substituted by one or more R^{B};
   R^{B} is oxo, halo, cyano, nitro, hydroxy or a group:

—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl;
(50) m is 0;
(51) m is 1;
(52) R₄ is selected from halo, cyano, nitro, hydroxy or a group

—Y⁴—X⁴—Z⁴
wherein
Y⁴ is absent or a linker group of the formula —[CR^{4A}R^{4B}]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring;
and wherein any alkyl, aryl, heterocyclyl or heteroaryl group present in a substituent group on Z⁴ is optionally further substituted by;
(i) halo, cyano, nitro, hydroxy, caboxy, NR^{4G}R^{4H}, (1-2C)alkoxy, or (1-2C)alkyl; wherein R^{4G} and R^{4H} are selected from hydrogen or (1-2C)alkyl; or
(ii) oxo, halo, cyano, nitro, hydroxy, carboxy, NR^{4G}R^{4H}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4G}, OC(O)R^{4H}, C(O)NR^{4G}R^{4H}, NR^{4G}C(O)R^{4H}, NR^{4G}S(O)₂R^{4H} and S(O)₂NR^{4G}R^{4H}; wherein R^{4G} and R^{4H} are each independently selected from hydrogen, (1-4C)alkyl or (3-6C)cycloalkyl or (3-6C)cycloalkyl(1-2C)alkyl;
(53) R₄ is selected from halo, cyano, nitro, hydroxy or a group

—Y⁴—X⁴—Z⁴
wherein
Y⁴ is absent or a linker group of the formula —[CR^{4A}R^{4B}]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring;
   and wherein any alkyl, aryl, heterocyclyl or heteroaryl group present in a substituent group on Z⁴ is optionally further substituted by; oxo, halo, cyano, nitro, hydroxy, carboxy, NR^{4G}R^{4H}, (1-4C)alkoxy, (1-4C)alkyl, (3-8C)cycloalkyl, (1-4C)alkanoyl, (1-4C)alkylsulphonyl, Si[(1-4C)alkyl]₃, C(O)OR^{4G}, OC(O)R^{4H}, C(O)NR^{4G}R^{4H}, NR^{4G}C(O)R^{4H}, NR^{4G}S(O)₂R^{4H} and S(O)₂NR^{4G}R^{4H}; wherein R^{4G} and R^{4H} are each independently selected from hydrogen, (1-4C)alkyl or (3-6C)cycloalkyl or (3-6C)cycloalkyl(1-2C)alkyl;
(54) R₄ is selected from halo, cyano, nitro, hydroxy or a group

—Y⁴—X⁴—Z⁴
wherein
Y⁴ is absent or a linker group of the formula —[CR^{4A}R^{4B}]ₚ— in which p is an integer selected from 1 or 2, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl;
and wherein any alkyl, aryl, heterocyclyl or heteroaryl group present in a substituent group on Z⁴ is optionally further substituted by; oxo, halo, cyano, hydroxy, carboxy, NR^{4G}R^{4H}, (1-4C)alkoxy, (1-4C)alkyl, (3-8C)cycloalkyl, (1-4C)alkanoyl, (1-4C)alkylsulphonyl, Si[(1-4C)alkyl]₃, C(O)OR^{4G}, OC(O)R^{4H}, C(O)NR^{4G}R^{4H}, NR^{4G}C(O)R^{4H}, NR^{4G}S(O)₂R^{4H} and S(O)₂NR^{4G}R^{4H}; wherein R^{4G} and R^{4H} are each independently selected from hydrogen or (1-2C)alkyl;
(55) R₄ is selected from halo, cyano, nitro, hydroxy or a group

—Y⁴—X⁴—Z⁴
wherein
Y⁴ is absent or a linker group of the formula —[CR^{4A}R^{4B}]ₚ— in which p is an integer selected from 1 or 2, and R^{4A} and R^{4B} are each independently selected from hydrogen or methyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, phenyl, (3-6C)cycloalkyl, 5 or 6-membered heteroaryl or a 4 to 7-membered heterocyclyl; and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, phenyl, phenyloxy, phenyl-(1-2C)alkyl, 4 to 7-membered heterocyclyl, 4 to 7-membered heterocyclyloxy, 4 to 7-membered heterocyclyl-(1-2C)alkyl, 5 or 6-membered heteroaryl, 4 to 7-membered heteroaryloxy, 5 or 6-membered heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl;
(56) R₄ is selected from halo, cyano, hydroxy or a group

—Y⁴—X⁴—Z⁴
wherein
Y⁴ is absent or a linker group of the formula —[CR^{4A}R^{4B}]ₚ— in which p is an integer selected from 1 or 2, and R^{4A} and R^{4B} are each independently selected from hydrogen or methyl;
X⁴ is absent or —O—, —C(O)—, —C(O)O—, —OC(O)—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, phenyl, (3-6C)cycloalkyl, 5 or 6-membered heteroaryl or a 4 to 7-membered heterocyclyl; and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, phenyl, 4 to 7-membered heterocyclyl, 5 or 6-membered heteroaryl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen or (1-4C)alkyl;
(57) n is 0;
(58) n is 1;
(59) n is 2;
(60) n is 0 or 1 and R₅ is selected from halo, cyano, hydroxy or a group

—Y⁵—X⁵—Z⁵
wherein:
Y⁵ is absent or a linker group of the formula —[CR^{5A}R^{5B}]_{q}— in which q is an integer selected from 1 or 2 and R^{5A} and R^{5B} are each independently selected from hydrogen or methyl;
X⁵ is absent or —O—, —C(O)—, —C(O)O—, —OC(O)—, —CH(OR^{5C})—, —N(R^{5C})—, —N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, hydroxy, carboxy, NR^{5E}R^{5F} or (1-2C)alkoxy; wherein R^{5E} and R^{5F} are each independently selected from hydrogen or (1-2C)alkyl; or
n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, hydroxy or a group

—Y⁶—X⁶—Z⁶
wherein:
Y⁶ is absent or a linker group of the formula —[CR^{6A}R^{6B}]_{q}— in which q is an integer selected from 1 or 2 and R^{6A} and R^{6B} are each independently selected from hydrogen or methyl;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or (1-2C)alkyl.
(61) n is 0 or 1 and R₅ is selected from halo, cyano, hydroxy or a group

—Y⁵—X⁵—Z⁵
wherein:
Y⁵ is absent or a linker group of the formula —[CR^{5A}R^{5B}]_{q}— in which q is 1 and R^{5A} and R^{5B} are each independently selected from hydrogen or methyl;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, hydroxy, carboxy, NR^{5E}R^{5F} or (1-2C)alkoxy; wherein R^{5E} and R^{5F} are each independently selected from hydrogen or methyl; or
n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, hydroxy or a group

—Y⁶—X⁶—Z⁶
wherein:
Y⁶ is absent or a linker group of the formula —[CR^{6A}R^{6B}]_{q}— in which q is 1 and R^{6A} and R^{6B} are each independently selected from hydrogen or methyl;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or methyl.
(62) n is 0 or 1 and R₅ is selected from halo, cyano, hydroxy or a group

—Y⁵—X⁵—Z⁵
wherein:
Y⁵ is absent;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, hydroxy, carboxy, NR^{5E}R^{5F} or (1-2C)alkoxy; wherein R^{5E} and R^{5F} are each independently selected from hydrogen or methyl; or
n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, hydroxy or a group

—Y⁶—X⁶—Z⁶
wherein:
Y⁶ is absent;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or methyl.
(63) n is 0 or 1 and R₅ is selected from halo, cyano, hydroxy or a group

—Y⁵—X⁵—Z⁵
wherein:
Y⁵ is absent;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, hydroxy, carboxy, NR^{5E}R^{5F} or (1-2C)alkoxy; wherein R^{5E} and R^{5F} are each independently selected from hydrogen or methyl;

Suitably, a heteroaryl or heterocyclyl group as defined herein is a monocyclic or bicyclic heteroaryl or heterocyclyl group comprising one, two or three heteroatoms selected from N, O or S. More suitably, a heteroaryl or heterocyclyl group as defined herein is a monocyclic heteroaryl or heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5-, 6- or 7-membered heterocyclyl ring comprising one, two or three heteroatoms selected from N, O or S. Most suitably, a heterocyclyl group is a 5-, 6- or 7-membered ring comprising one, two or three heteroatoms selected from N, O or S [e.g. morpholinyl (e.g. 4-morpholinyl), pyridinyl, piperazinyl, homopiperazinyl or pyrrolidinonyl].

Suitably an aryl group is phenyl.

Suitably, A₁ is as defined in any one of paragraphs (1) to (8) above. In an embodiment, A₁ is as defined in paragraph (3).

Suitably, A₂ is as defined in any one of paragraphs (9) to (16) above. In an embodiment, A₁ is as defined in paragraph (11).

Suitably, A₃ is as defined in any one of paragraphs (17) to (24) above. In an embodiment, A₃ is as defined in paragraph (17) or (19).

Suitably, R₁ is as defined in any one of paragraphs (27) to (36) above. More suitably, R₁ is as defined in paragraphs (35) or (36). Most suitably, R₁ is H.

Suitably, R₂ is as defined in any one of paragraphs (37) to (44) above. More suitably, R₂ is as defined in any one of paragraphs (43) or (44). Most suitably, R₂ is C(O)OH or tetrazolyl.

Suitably, R₃ is as defined in any one of paragraphs (45) to (49) above. More suitably, R₃ is as defined in any one of paragraphs (48) or (49). Most suitably, R₃ is as defined in paragraph (49). In an embodiment, R₃ is as defined in any one of paragraphs (45) to (49) above, with the proviso that R₃ is not hydrogen.

Suitably, m is 0 or 1. In an embodiment, m is 1.

Suitably, R₄ is as defined in any one of paragraphs (52) to (56) above. More suitably, R₄ is as defined in any one of paragraphs (55) or (56).

Suitably, n is 0 or 2. In an embodiment, n is 0.

Suitably, n and R₅ are as defined in any one of paragraphs (60) to (63) above. More suitably, n and R₅ are as defined in any one of paragraphs (61) or (62). Most suitably, n and R₅ are as defined in paragraph (62).

In a particular group of compounds of the invention, R₁ is H, i.e. the compounds have the structural formula Ia (a sub-definition of formula I) shown below:

[chemical structure]

wherein A₁, A₂, A₃, R₂, R₃, m, R₄, n and R₅ each have any one of the meanings defined herein; or a pharmaceutically acceptable salt, hydrate and/or solvate thereof.

In an embodiment of the compounds of formula Ia:

A₁ is as defined in any one of paragraphs (1) to (8) above;

A₂ is as defined in any one of paragraphs (9) to (16) above;

A₃ is as defined in any one of paragraphs (17) to (24) above;

R₂ is as defined in any one of paragraphs (37) to (44) above;

R₃ is as defined in any one of paragraphs (45) to (49) above;

m is 0 or 1;

R₄ is as defined in any one of paragraphs (52) to (56) above; and

n and R₅ are as defined in any one of paragraphs (60) to (63) above.

In another embodiment of the compounds of formula Ia:

A₁ is as defined in paragraph (1) or (3) above;

A₂ is as defined in paragraph (11);

A₃ is as defined in paragraph (17) or (19) above;

R₂ is as defined in paragraph (44) above;

R₃ is as defined in paragraph (49) above;

m is 0 or 1;

R₄ is as defined in paragraph (55) or (56) above; and

n and R₅ are as defined in paragraph (62) above.

In another embodiment of the compounds of formula Ia:

A₁ is as defined in paragraph (1) above;

A₂ is as defined in paragraph (11);

A₃ is as defined in paragraph (19) above;

R₂ is as defined in paragraph (44) above;

R₃ is as defined in paragraph (49) above;

m is 0 or 1;

R₄ is as defined in paragraph (56) above; and

n and R₅ are as defined in paragraph (62) above.

In a particular group of compounds of the Formula Ia, R₂ is C(O)OH or tetrazolyl, and A₁, A₂, A₃, R₃, m, R₄, n and R₅ each have any one of the meanings defined herein; or a pharmaceutically acceptable salt, hydrate and/or solvate thereof.

In a particular group of compounds of the Formula Ia, wherein R₂, R₃, m, R₄, n and R₅ each have any one of the meanings defined herein and A₁, A₂ and A₃ are selected from one of the following options:

(i) A₁ is C, A₂ is C and A₃ is C;

(ii) A₁ is N, A₂ is C and A₃ is C;

(iii) A₁ is S, A₂ is C and A₃ is C;

(iv) A₁ is O, A₂ is C and A₃ is C;

(v) A₁ is C, A₂ is C and A₃ is S;

(vi) A₁ is C, A₂ is C and A₃ is S(O)₂;

(vii) A₁ is N, A₂ is N and A₃ is C;

(viii) A₁ is N, A₂ is C and A₃ is N.

A particular group of compounds of the invention have one of the following structural formulae;

[chemical structure]

wherein R₂, R₃, m, R₄, n and R₅ each have any one of the meanings defined herein; or a pharmaceutically acceptable salt, hydrate and/or solvate thereof.

In an embodiment of the compounds of formulae (III), (IV), (V), (VI), (VII):

R₂ is as defined in any one of paragraphs (37) to (44) above;

R₃ is as defined in any one of paragraphs (45) to (49) above;

m is 0 or 1;

R₄ is as defined in any one of paragraphs (52) to (56) above; and

n and R₅ are as defined in any one of paragraphs (60) to (63) above.

In an embodiment of the compounds of formulae (III), (IV), (V), (VI) or (VII):

R₂ is as defined in paragraph (44) above;

R₃ is as defined in paragraph (49) above;

m is 0 or 1;

R₄ is as defined in paragraph (55) or (56) above; and

n and R₅ are as defined in paragraph (62) above.

In an embodiment of the compounds of formulae (III), (IV), (V), (VI) or (VII):

R₂ is as defined in paragraph (44) above;

R₃ is as defined in paragraph (49) above;

m is 0 or 1;

R₄ is as defined in paragraph (56) above; and

n and R₅ are as defined in paragraph (62) above.

In an embodiment of the compounds of formulae (III), (IV), (V), (VI) or (VII), R₂ is C(O)OH or tetrazolyl, and A₁, A₂, A₃, R₃, m, R₄, n and R₅ each have any one of the meanings defined herein; or a pharmaceutically acceptable salt, hydrate and/or solvate thereof.

In an embodiment of the compounds of formula (VIII):

R₂ is as defined in any one of paragraphs (37) to (44) above;

R₃ is as defined in any one of paragraphs (45) to (49) above;

R₄ is as defined in any one of paragraphs (52) to (56) above; and

R₅ is as defined in any one of paragraphs (60) to (63) above.

In an embodiment of the compounds of formula (VIII):

R₂ is as defined in paragraph (44) above;

R₃ is as defined in paragraph (49) above;

R₄ is as defined in paragraph (55) or (56) above; and

R₅ is as defined in paragraph (62) above.

In an embodiment of the compounds of formula (VIII):

R₂ is as defined in paragraph (44) above;

R₃ is as defined in paragraph (49) above;

R₄ is as defined in paragraph (56) above; and

R₅ is as defined in paragraph (62) above.

In an embodiment of the compounds of formula (VIII), R₂ is C(O)OH or tetrazolyl, and A₁, A₂, R₃, R₄ and R₅ each have any one of the meanings defined herein; or a pharmaceutically acceptable salt, hydrate and/or solvate thereof.

In another embodiment, the compounds are of formula (II) defined herein.

In another embodiment, the compounds are of formula (III) defined herein.

In another embodiment, the compounds are of formula (IV) defined herein.

In another embodiment, the compounds are of formula (V) defined herein.

In another embodiment, the compounds are of formula (VI) defined herein.

In another embodiment, the compounds are of formula (VII) defined herein.

In another embodiment, the compounds are of formula (VIII) defined herein.

Particular compounds of the present invention include any of the compounds exemplified in the present application, or a pharmaceutically acceptable salt or solvate thereof, and, in particular, any of the following:

[chemical structure]

3-Bromo-6-(3,5-dichlorophenyl)-4H-thieno[3,2-b]pyrrole-5-carboxylic acid;

[chemical structure]

3-Cyclopropyl-6-(3,5-dichlorophenyl)-4H-thieno[3,2-b]pyrrole-5-carboxylic acid;

[chemical structure]

3-Bromo-4H-thieno[3,2-b]pyrrole-5-carboxylic acid;

[chemical structure]

3-Bromo-6-(4-fluorophenyl)-4H-thieno-[3,2-b]pyrrole-5-carboxylic acid;

[chemical structure]

3,6-Bis(4-fluorophenyl)-4H-thieno-[3,2-b]pyrrole-5-carboxylic acid;

[chemical structure]

3-Isopropenyl-6-(4-fluorophenyl)-4H-thieno-[3,2-b]pyrrole-5-carboxylic acid;

[chemical structure]

3-Isopropyl-4H-thieno[3,2-b]pyrrole-5-carboxylic acid;

[chemical structure]

3-Cyclopropyl-4H-thieno[3,2-b]pyrrole-5-carboxylic acid;

[chemical structure]

6-(3-Acetamidophenyl)-3-bromo-4H-thieno[3,2-b]pyrrole-5-carboxylic acid;

[chemical structure]

3-Isopropyl-6-(4-fluorophenyl)-4H-thieno-[3,2-b]pyrrole-5-carboxylic acid;

[chemical structure]

3-Cyclopropyl-6-(4-fluorophenyl)-4H-thieno-[3,2-b]pyrrole-5-carboxylic acid;

[chemical structure]

3-bromo-6-(4-sulfamoylphenyl)-4H-thieno-[3,2-b]pyrrole-5-carboxylic acid;

[chemical structure]

3-Cyano-6-(4-fluorophenyl)-4H-thieno-[3,2-b]pyrrole-5-carboxylic acid;

[chemical structure]

6-(4-fluorophenyl)-4H-thieno-[3,2-b]pyrrole-3,5-dicarboxylic acid;

[chemical structure]

4-(3,5-dichlorophenyl)-6H-thieno[2,3-b]pyrrole-5-carboxylic acid;

Further compounds of the present invention include any one of the following, or a pharmaceutically acceptable salt or solvate thereof:

[chemical structure]

3-(1-Acetamidoethyl)-6-(3,5-dichlorophenyl)-4H-thieno[3,2-b]pyrrole-5-carboxylic acid;

[chemical structure]

6-(3-Chloro-4-((methylsulfonyl)methyl)phenyl)-3-isopropyl-4H-thieno[3,2-b]pyrrole-5-carboxylic acid;

[chemical structure]

3-Bromo-6-(3,5-dichlorophenyl)-5-(1H-tetrazol-5-yl)-4H-thieno[3,2-b]pyrrole;

[chemical structure]

6-(3,5-Dichlorophenyl)-3-isopropyl-5-(1H-tetrazol-5-yl)-4H-thieno[3,2-b]pyrrole;

[chemical structure]

6-(3,5-Dichlorophenyl)-3-methoxy-4H-thieno[3,2-b]pyrrole-5-carboxylic acid;

[chemical structure]

6-(3,5-Dichlorophenyl)-3-phenethoxy-4H-thieno[3,2-b]pyrrole-5-carboxylic acid;

[chemical structure]

3-acetyl-6-(3,5-Dichlorophenyl)-4H-thieno[3,2-b]pyrrole-5-carboxylic acid;

[chemical structure]

6-(3,5-Dichlorophenyl)-3-(1-hydroxyethyl)-4H-thieno[3,2-b]pyrrole-5-carboxylic acid;

[chemical structure]

6-(3,5-Dichlorophenyl)-3-(1-isopropoxyethyl)-4H-thieno[3,2-b]pyrrole-5-carboxylic acid;

[chemical structure]

3-(1-(Benzyloxy)ethyl)-6-(3,5-dichlorophenyl)-4H-thieno[3,2-b]pyrrole-5-carboxylic acid;

[chemical structure]

6-(3,5-Dichlorophenyl)-3-(1-(dimethylamino)ethyl)-4H-thieno[3,2-b]pyrrole-5-carboxylic acid;

[chemical structure]

3-(1-Acetamidovinyl)-6-(3,5-dichlorophenyl)-4H-thieno[3,2-b]pyrrole-5-carboxylic acid;

[chemical structure]

3-(1-Acetamidoethyl)-6-(3,5-dichlorophenyl)-4H-thieno[3,2-b]pyrrole-5-carboxylic acid;

[chemical structure]

6-(3,5-Dichlorophenyl)-3-(1-(methylsulfonamido)ethyl)-4H-thieno[3,2-b]pyrrole-5-carboxylic acid;

[chemical structure]

6-(3,5-Dichlorophenyl)-3-vinyl-4H-thieno[3,2-b]pyrrole-5-carboxylic acid;

[chemical structure]

6-(3,5-Dichlorophenyl)-3-(methylsulfonyl)-4H-thieno[3,2-b]pyrrole-5-carboxylic acid;

[chemical structure]

3-(Azetidin-3-ylsulfonyl)-6-(3,5-dichlorophenyl)-4H-thieno[3,2-b]pyrrole-5-carboxylic acid;

[chemical structure]

6-(4-Fluorophenyl)-3-(piperidin-4-yl)-4H-thieno[3,2-b]pyrrole-5-carboxylic acid;

[chemical structure]

6-(4-Fluorophenyl)-3-(1,2,3,6-tetrahydropyridin-4-yl)-4H-thieno[3,2-b]pyrrole-5-carboxylic acid;

[chemical structure]

3-(1-(tert-Butoxycarbonyl)-1,2,3,6-tetrahydropyridin-4-yl)-6-(4-fluorophenyl)-4H-thieno[3,2-b]pyrrole-5-carboxylic acid;

[chemical structure]

6-(4-Fluorophenyl)-3-(1H-tetrazol-5-yl)-4H-thieno[3,2-b]pyrrole-5-carboxylic acid;

[chemical structure]

3-Bromo-6-(3-((tert-butoxycarbonyl)amino)phenyl)-4H-thieno[3,2-b]pyrrole-5-carboxylic acid;

[chemical structure]

6-(3-Aminophenyl)-3-bromo-4H-thieno[3,2-b]pyrrole-5-carboxylic acid hydrochloride;

[chemical structure]

6-(1H-Benzo[d]imidazol-7-yl)-3-bromo-4H-thieno[3,2-b]pyrrole-5-carboxylic acid;

[chemical structure]

6-(1H-Benzo[d]imidazol-7-yl)-3-isopropyl-4H-thieno[3,2-b]pyrrole-5-carboxylic acid;

[chemical structure]

6-(2-Amino-1H-benzo[d]imidazol-7-yl)-3-bromo-4H-thieno[3,2-b]pyrrole-5-carboxylic acid;

[chemical structure]

6-(2-Amino-1H-benzo[d]imidazol-7-yl)-3-isopropyl-4H-thieno[3,2-b]pyrrole-5-carboxylic acid;

[chemical structure]

6-(2-Amino-1H-benzo[d]imidazol-7-yl)-3-(1-(methylsulfonamido)ethyl)-4H-thieno[3,2-b]pyrrole-5-carboxylic acid;

[chemical structure]

6-(3,5-Dichlorophenyl)-3-(piperidin-4-yloxy)-4H-thieno[3,2-b]pyrrole-5-carboxylic acid;

[chemical structure]

4-(3-Chloro-4-((methylsulfonyl)methyl)phenyl)-6H-thieno[2,3-b]pyrrole-5-carboxylic acid;

[chemical structure]

4-(3,5-Dichlorophenyl)-2-isopropyl-6H-thieno[2,3-b]pyrrole-5-carboxylic acid;

[chemical structure]

4-(4-(N,N-Dimethylsulfamoyl)phenyl)-2-isobutyl-6H-thieno[2,3-b]pyrrole-5-carboxylic acid;

[chemical structure]

4-(3,5-Dichlorophenyl)-6H-thieno[2,3-b]pyrrole-5-carboxylic acid 1,1-dioxide;

[chemical structure]

3-Bromo-4H-furo[3,2-b]pyrrole-5-carboxylic acid;

[chemical structure]

3-Bromo-6-(3,5-dichlorophenyl)-4H-furo[3,2-b]pyrrole-5-carboxylic acid;

[chemical structure]

3-Cyclopropyl-6-(3,5-dichlorophenyl)-4H-furo[3,2-b]pyrrole-5-carboxylic acid;

[chemical structure]

6-(3,5-Dichlorophenyl)-3-(prop-1-en-2-yl)-4H-furo[3,2-b]pyrrole-5-carboxylic acid;

[chemical structure]

6-(3,5-Dichlorophenyl)-3-isopropyl-4H-furo[3,2-b]pyrrole-5-carboxylic acid;

[chemical structure]

3-Cyclopropyl-1,4-dihydropyrrolo[3,2-c]pyrazole-5-carboxylic acid;

[chemical structure]

3-Cyclopropyl-1-((2-(trimethylsilyl)ethoxy)methyl)-1,4-dihydropyrrolo[3,2-c]pyrazole-5-carboxylic acid;

[chemical structure]

3-Cyclopropyl-6-(3,5-dichlorophenyl)-1-((2-(trimethylsilyl)ethoxy)methyl)-1,4-dihydropyrrolo[3,2-c]pyrazole-5-carboxylic acid;

[chemical structure]

3-Cyclopropyl-6-(3,5-dichlorophenyl)-1,4-dihydropyrrolo[3,2-c]pyrazole-5-carboxylic acid;

[chemical structure]

3-Cyclopropyl-1-(N,N-dimethylsulfamoyl)-1,4-dihydropyrrolo[3,2-c]pyrazole-5-carboxylic acid;

[chemical structure]

3-Cyclopropyl-6-(3,5-dichlorophenyl)-1-(N,N-dimethylsulfamoyl)-1,4-dihydropyrrolo[3,2-c]pyrazole-5-carboxylic acid;

[chemical structure]

6-(3-Chloro-4-((methylsulfonyl)methyl)phenyl)-3-cyclopropyl-1-(N,N-dimethylsulfamoyl)-1,4-dihydropyrrolo[3,2-c]pyrazole-5-carboxylic acid;

[chemical structure]

(E)-3-(piperidin-4-yl)-6-styryl-3,4-dihydropyrrolo[2,3-d]imidazole-5-carboxylic acid;

[chemical structure]

3-(2-chlorobenzyl)-5,8-dihydro-1H,6H-pyrano[3′,4′:4,5]thieno[3,2-b]pyrrole-2-carboxylic acid;

[chemical structure]

3-(3-chlorophenyl)-8-isopropyl-1,8-dihydropyrrolo[2,3-b]indole-2-carboxylic acid;

[chemical structure]

3-(3,5-dichlorophenyl)-4-methyl-1,4-dihydropyrrolo[3,2-b]indole-2-carboxylic acid;

[chemical structure]

7-(2-aminoethoxy)-3-(3,5-dichlorophenyl)-1H-benzofuro[3,2-b]pyrrole-2-carboxylic acid;

[chemical structure]

6-(3,5-dichlorophenyl)-3-ethyl-2-methyl-4H-furo[3,2-b]pyrrole-5-carboxylic acid;

[chemical structure]

3,6-bis(3-chloro-4-((methylsulfonyl)methyl)phenyl)-4H-thieno[3,2-b]pyrrole-5-carboxylic acid;

[chemical structure]

6-(3-chloro-4-((methylsulfonyl)methyl)phenyl)-3-cyclopropyl-4H-thieno[3,2-b]pyrrole-5-carboxylic acid;

[chemical structure]

(E)-6-(3-chloro-4-((methylsulfonyl)methyl)phenyl)-3-(1-((2-(dimethylamino)ethoxy)imino)ethyl)-4H-thieno[3,2-b]pyrrole-5-carboxylic acid;

[chemical structure]

6-(3-chloro-4-((methylsulfonyl)methyl)phenyl)-4H-thieno[3,2-b]pyrrole-5-carboxylic acid;

[chemical structure]

3-acetyl-6-(3,5-dichlorophenyl)-4H-thieno[3,2-b]pyrrole-5-carboxylic acid;

[chemical structure]

6-acetyl-3-(3,5-dichlorophenyl)-2-methyl-4H-thieno[3,2-b]pyrrole-5-carboxylic acid

[chemical structure]

6-acetyl-3-(3,5-dichlorophenyl)-4H-thieno[3,2-b]pyrrole-5-carboxylic acid;

[chemical structure]

6-(3,5-dichlorophenyl)-3-(1H-tetrazol-5-yl)-4H-thieno[3,2-b]pyrrole-5-carboxylic acid;

[chemical structure]

3-(1-(tert-butoxycarbonyl)-1,2,3,6-tetrahydropyridin-4-yl)-6-(3,5-dichlorophenyl)-4H-thieno[3,2-b]pyrrole-5-carboxylic acid;

[chemical structure]

3-(1-(tert-butoxycarbonyl)piperidin-4-yl)-6-(3,5-dichlorophenyl)-4H-thieno[3,2-b]pyrrole-5-carboxylic acid;

[chemical structure]

6-(3,5-dichlorophenyl)-3-(5-(hydroxymethyl)-1,2,4-oxadiazol-3-yl)-4H-thieno[3,2-b]pyrrole-5-carboxylic acid

[chemical structure]

6-(3,5-dichlorophenyl)-3-(1-(methylsulfonyl)piperidin-4-yl)-4H-thieno[3,2-b]pyrrole-5-carboxylic acid;

[chemical structure]

6-(3,5-dichlorophenyl)-3-(piperidin-4-yl)-4H-thieno[3,2-b]pyrrole;

[chemical structure]

2-benzyl-6-(3-chloro-4-((methylsulfonyl)methyl)phenyl)-3-isopropyl-1-methyl-1,2-dihydropyrrolo[3,2-c]pyrazole-5-carboxylic acid;

[chemical structure]

3-isopropyl-2-methyl-6-(4-((methylsulfonyl)methyl)phenyl)-2,4-di hydropyrrolo[3,2-c]pyrazole-5-carboxylic acid;

[chemical structure]

3-isopropyl-1-methyl-6-(4-((methylsulfonyl)methyl)phenyl)-1,4-di hydropyrrolo[3,2-c]pyrazole-5-carboxylic acid;

[chemical structure]

6-(3-chloro-4-((methylsulfonyl)methyl)phenyl)-3-isopropyl-1,2-dimethyl-1,2-dihydropyrrolo[3,2-c]pyrazole-5-carboxylic acid;

[chemical structure]

6-(3-chloro-4-((methylsulfonyl)methyl)phenyl)-3-isopropyl-1-methyl-1,4-dihydropyrrolo[3,2-c]pyrazole-5-carboxylic acid;

[chemical structure]

6-(3-chloro-4-((methylsulfonyl)methyl)phenyl)-3-isopropyl-2-methyl-2,4-dihydropyrrolo[3,2-c]pyrazole-5-carboxylic acid;

[chemical structure]

2-benzyl-6-(3-chloro-4-((methylsulfonyl)methyl)phenyl)-3-cyclopropyl-2,4-dihydropyrrolo[3,2-c]pyrazole-5-carboxylic acid;

[chemical structure]

3-cyclopropyl-6-(4-((methylsulfonyl)methyl)phenyl)-2,4-di hydropyrrolo[3,2-c]pyrazole-5-carboxylic acid;

[chemical structure]

6-(3-chloro-4-((methylsulfonyl)methyl)phenyl)-3-cyclopropyl-1,4-dimethyl-1,4-dihydropyrrolo[3,2-c]pyrazole-5-carboxylic acid;

[chemical structure]

1-benzyl-3-cyclopropyl-1,4-dihydropyrrolo[3,2-c]pyrazole-5-carboxylic acid;

[chemical structure]

3-cyclopropyl-6-(4-fluorophenyl)-1,4-dihydropyrrolo[3,2-c]pyrazole-5-carboxylic acid;

[chemical structure]

3-cyclopropyl-6-(4-fluorophenyl)-2-methyl-2,4-di hydropyrrolo[3,2-c]pyrazole-5-carboxylic acid; or

[chemical structure]

3-cyclopropyl-6-(4-fluorophenyl)-1-methyl-1,4-dihydropyrrolo[3,2-c]pyrazole-5-carboxylic acid.

The various functional groups and substituents making up the compounds of the formula I are typically chosen such that the molecular weight of the compound of the formula I does not exceed 1000. More usually, the molecular weight of the compound will be less than 900, for example less than 800, or less than 700, or less than 650, or less than 600. More preferably, the molecular weight is less than 550 and, for example, is 500 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn-Ingold-Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For example, H may be in any isotopic form, including ²H(D), and ³H (T); C may be in any isotopic form, including ¹²C, ¹³C, and ¹⁴C; N may be in any isotopic form, including ⁷N and ⁸N (i.e. nitrogen-14 and nitrogen-15); and O may be in any isotopic form, including ¹⁶O and ¹⁸O; and the like.

It is also to be understood that certain compounds of the formula I may exist in solvated as well as unsolvated forms such as, for example, hydrated forms. It is to be understood that the invention encompasses all such solvated forms that possess antiproliferative activity.

It is also to be understood that certain compounds of the formula I may exhibit polymorphism, and that the invention encompasses all such forms that possess antiproliferative activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

[chemical structure]

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula I may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula I and in-vivo cleavable amide derivatives that may be formed at a carboxy group or an amino group in a compound of the formula I.

Accordingly, the present invention includes those compounds of the formula I as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human, animal or bacterial body by way of metabolism of a precursor compound, that is a compound of the formula I may be a synthetically-produced compound or a metabolically-produced compound.

A suitable pharmaceutically acceptable pro-drug of a compound of the formula I is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C₁₋₆ alkyl esters such as methyl, ethyl and tert-butyl, C₁₋₆alkoxymethyl esters such as methoxymethyl esters, C₁₋₆alkanoyloxymethyl esters such as pivaloyloxymethyl esters, 3-phthalidyl esters, C₃₋₈cycloalkylcarbonyloxy-C₁₋₆alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆alkoxycarbonyloxy-C₁₋₆ alkyl esters such as methoxycarbonyloxymethyl and 1-methoxycarbonyloxyethyl esters.

A suitable pharmaceutically acceptable pro-drug of a compound of the formula I that possesses a hydroxy group is, for example, an in vivo cleavable ester or ether thereof. An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula I that possesses a carboxy group is, for example, an in vivo cleavable amide thereof, for example an amide formed with an amine such as ammonia, a C₁₋₄alkylamine such as methylamine, a (C₁₋₄alkyl)₂amine such as dimethylamine, N-ethyl-N-methylamine or diethylamine, a C₁₋₄alkoxy-C₂₋₄ alkylamine such as 2-methoxyethylamine, a phenyl-C₁₋₄alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C₁₋₁₀alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl.

The in vivo effects of a compound of the formula I may be exerted in part by one or more metabolites that are formed within the human or animal body after administration of a compound of the formula I. As stated hereinbefore, the in vivo effects of a compound of the formula I may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of formula I will vary depending on the nature of A₁, A₂, A₃, A₄, R₁, R₂, R₃, R₄, R₅, R₆, R₇ and any substituent groups associated therewith. Suitable processes for their preparation are described further in the accompanying Examples.

Once a compound of formula I has been synthesised by any one of the processes defined herein, the processes may then further comprise the additional steps of:

removing any protecting groups present;

(ii) converting the compound formula I into another compound of formula I;
(iii) forming a pharmaceutically acceptable salt, hydrate or solvate thereof; and/or
(iv) forming a prodrug thereof.

An example of (ii) above is when a compound of formula I is synthesised and then one or more of the groups R₁, R₂, R₃, R₄ and R₅ may be further reacted to change the nature of the group and provide an alternative compound of formula I. For example, the compound can be reacted to covert R₁ into a substituent group other than hydrogen.

The resultant compounds of formula I can be isolated and purified using techniques well known in the art.

### Biological Activity

The enzyme and in-vitro cell-based assays described in accompanying Example section, or elsewhere in the literature, may be used to measure the pharmacological effects of the compounds of the present invention.

Although the pharmacological properties of the compounds of formula I vary with structural change, as expected, the compounds of the invention were found to be active in these enzyme assays.

The compounds of the invention demonstrate a pIC₅₀ of 4 or more in the enzyme assays described herein, with preferred compounds of the invention demonstrating an pIC₅₀ of 4.5 or more and the most preferred compounds of the invention demonstrating an pIC₅₀ of 5 or more.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier. For example, solid oral forms may contain, together with the active compound, diluents, such as, for example, lactose, dextrose, saccharose, cellulose, corn starch or potato starch; lubricants, such as, for example, silica, talc, stearic acid, magnesium or calcium stearate, and/or polyethylene glycols; binding agents; such as, for example, starches, arabic gums, gelatin, methylcellulose, carboxymethylcellulose or polyvinyl pyrrolidone; disaggregating agents, such as, for example, starch, alginic acid, alginates or sodium starch glycolate; effervescing mixtures; dyestuffs; sweeteners; wetting agents, such as, for example, lecithin, polysorbates, laurylsulphates; and, in general, non toxic and pharmacologically inactive substances used in pharmaceutical formulations. Such pharmaceutical compositions may be manufactured in by conventional methods known in the art, such as, for example, by mixing, granulating, tableting, sugar coating, or film coating processes.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing). Suitably, oral or parenteral administration is preferred. Most suitably, oral administration is preferred.

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the condition, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The compounds of the present invention are inhibitors of metallo-beta-lactamases (MBLs). Many bacteria have developed resistance to β-lactam antibacterials (BLAs) and one of the main resistance mechanisms is the hydrolysis of BLAs by MBLs. Thus, the inhibition of bacterial MBLs by the compounds of the present invention can significantly enhance the activity of BLAs, when administered with a compound of the present invention.

The present invention provides compounds that function as inhibitors of metallo-beta-lactamases.

The present invention therefore provides a method of inhibiting bacterial metallo-beta-lactamase activity in vitro or in vivo, said method comprising contacting a cell with an effective amount of a compound, or a pharmaceutically acceptable salt, hydrate or solvate thereof, or a pharmaceutical composition as defined herein.

The present invention also provides a method for the prevention or treatment of bacterial infection in a patient or animal in need of such treatment, said method comprising administering to said patient a therapeutically effective amount of a compound, or a pharmaceutically acceptable salt, hydrate or solvate thereof, or a pharmaceutical composition as defined herein, in combination with a suitable antibacterial agent.

In a preferred embodiment, the antibacterial agent is a β-lactam antibacterial agent, or analogue thereof. Non limiting examples of suitable β-lactam antibacterial agents include carbapenems (e.g. meropenem, faropenem, imipenem, ertapenem, doripenem, panipenem/betamipron and biapenem as well as razupenem, tebipenem, lenapenem and tomopenem), ureidopenicillins (e.g. piperacillin), carbacephems (e.g. loracarbef) and cephalosporins (e.g. cefpodoxime, ceftazidime, cefotaxime, ceftriaxone, ceftobiprole, and ceftaroline). Specific examples of suitable β-lactam antibacterial agents include, for example, temocillin, piperacillin, cefpodoxime, ceftazidime, cefotaxime, ceftriaxone, meropenem, faropenem, imipenem, loracarbef, ceftobiprole and ceftaroline.

The present invention also provides a method of inhibiting bacterial infection, in vitro or in vivo, said method comprising contacting a cell with an effective amount of a compound, or a pharmaceutically acceptable salt, hydrate or solvate thereof, as defined herein, in combination with a suitable antibacterial agent.

The present invention also provides a compound, or a pharmaceutically acceptable salt, hydrate or solvate thereof, or a pharmaceutical composition as defined herein for use in therapy.

The present invention also provides a compound, or a pharmaceutically acceptable salt, hydrate or solvate thereof, or a pharmaceutical composition as defined herein for use in the treatment of a bacterial infection. In one embodiment, the treatment may be prophylactic (i.e. intended to prevent disease).

The present invention provides a compound, or a pharmaceutically acceptable salt, hydrate or solvate thereof, as defined herein for use in the inhibition of metallo-beta-lactamase activity.

Furthermore, the present invention provides a compound, or a pharmaceutically acceptable salt, hydrate or solvate thereof, as defined herein for use in the treatment of a disease or disorder in which metallo-beta-lactamase activity is implicated.

The present invention also provides a kit of parts comprising a compound, or a pharmaceutically acceptable salt, hydrate or solvate thereof, or a pharmaceutical composition as defined herein, and a BLA and/or a BLA linked to a formula (I) compound.

The term “bacterial infection” will be understood to refer to the invasion of bodily tissue by any pathogenic microorganisms that proliferate, resulting in tissue injury that can progress to disease. Suitably, the pathogenic microorganism is a bacteria.

The bacterial infection may be caused by Gram-negative or Gram-positive bacteria. For example, the bacterial infection may be caused by bacteria from one or more of the following families; Clostridium, Pseudomonas, Escherichia, Klebsiella, Enterococcus, Enterobacter, Serratia, Stenotrophomonas, Aeromonas, Morganella, Yersinia, Salmonella, Proteus, Pasteurella, Haemophilus, Citrobacter, Burkholderia, Brucella, Moraxella, Mycobacterium, Streptococcus or Staphylococcus. Particular examples include Clostridium, Pseudomonas, Escherichia, Klebsiella, Enterococcus, Enterobacter, Streptococcus and Staphylococcus. The bacterial infection may, for example, be caused by one or more bacteria selected from Moraxella catarrhalis, Brucella abortus, Burkholderia cepacia, Citrobacter species, Escherichia coli, Haemophilus Pneumonia, Klebsiella Pneumonia, Pasteurella multocida, Proteus mirabilis, Salmonella typhimurium, Clostridium difficile, Yersinia enterocolitica Mycobacterium tuberculosis, Staphylococcus aureus, group B streptococci, Streptococcus Pneumonia, and Streptococcus pyogenes, e.g. from E. coli and K. pneumoniae.

It will be understood by a person skilled in the art that the patient in need thereof is suitably a human, but may also include, but is not limited to, primates (e.g. monkeys), commercially farmed animals (e.g. horses, cows, sheep or pigs) and domestic pets (e.g. dogs, cats, guinea pigs, rabbits, hamsters or gerbils). Thus the patient in need thereof may be any mammal that is capable of being infected by a bacterium.

### Routes of Administration

The compounds of the present invention, or pharmaceutical compositions comprising these compounds, may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Diagnostic Uses

The compounds of the present invention, or pharmaceutical compositions comprising these compounds in combination with a suitable antibacterial agent, may also be used in methods for the detection of metallo-beta-lactamases. It will be appreciated that the compounds of formula (I) may be modified to enable various types of assays known is the literature, such as those using spectroscopic such as fluorescence or luminescence based methods. Thus, in one variation a sample containing bacteria which is suspected of expressing MBLs can be cultured (a) in the presence of a beta-lactam antibiotic agent; and (b) in the presence of the antibiotic combination of the invention. If the bacteria are seen to grow under conditions (a), this suggests that a beta-lactamase, able to hydrolyse the antibiotic agent, is causing resistance of the bacteria to the antibiotic agent. However, if the bacteria do not grow under condition (b), i.e. in the presence of compound of the present invention and a suitable antibacterial agent, then the beta-lactamases present have been inhibited. Such a result suggests that the beta-lactamases are metallo-beta-lactamases. The method can be used to determine whether bacteria express metallo-beta-lactamase enzymes.

### EXAMPLES

### Abbreviations

BLA β-Lactam antibacterials
ca. circa (about)

### DCM Dichloromethane

DIAD Diisopropyl azodicarboxylate

### DMF Dimethylformamide

### DMSO Dimethylsulfoxide

dppf 1,1′-Bis(diphenylphosphino)ferrocene
EDC 1-Ethyl-3-(3-dimethylaminopropyl)carbodiimide
ESI Electrospray ionization

### HOBt Hydroxybenzotriazole

HPLC High performance liquid chromatography

### IMP-1 Imipenemase-1

LCMS Liquid chromatography-mass spectrometry
MBL Metallo-beta-lactamase
MIC Minimum inhibitory concentration
MS Molecular sieves

### m/z Mass/charge

NDM-1 New Delhi Metallo-beta-lactamase-1

### NIS N-Iodosuccinimide

### NMR Nuclear Magnetic Resonance

PTSA p-Toluenesulfonic acid
ppm parts per million
RT Retention time
rt Room temperature
SCX-2 Strong cation exchange (Si-Propylsulfonic acid)

### TEA Triethylamine

### THF Tetrahydrofuran

### UV Ultraviolet

VIM Veronese metallo-β-lactamase
NDM New Delhi metallo-β-lactamase

### IMP-1 Imipenemase-1

### Materials and Methods

Standard experimental procedures were followed for synthesis; some of these are defined below.

Chemicals and solvents were from commonly used suppliers and were used without further purification. Silica gel 60 F254 analytical thin layer chromatography (TLC) plates were from Merck (Darmstadt, Germany) and visualized under UV light and/or with potassium permanganate stain. Chromatographic purifications were performed using Merck Geduran 60 silica (40-63 μm) or prepacked SNAP columns using a Biotage SP1 Purification system (Uppsala, Sweden). Microwave assisted reactions were performed using a Biotage Initiator™ microwave synthesizer in sealed vials. Deuterated solvents were obtained from Cambridge Isotopes, Sigma-Aldrich, Goss Scientific Instruments Ltd. and Apollo Scientific Ltd. All ¹H and ¹³C NMR spectra were recorded using a Bruker spectrometer. All chemical shifts are given in ppm relative to the solvent peak, and coupling constants (J) are reported in Hz. High Resolution (HR) mass spectrometry data (m/z) were obtained from a Bruker MicroTOF instrument using an ESI source and Time of Flight (TOF) analyzer. Low Resolution (LR) mass spectrometry data (m/z) were obtained from a Waters LCT Premier instrument using an ESI source and Time of Flight (TOF) analyzer or an Agilent Mass Spectrometer with a multimode source attached to an Agilent HPLC. Melting points were obtained using an automatic melting point apparatus.

### LCMS Analytical Methods

### Analytical Method A

LCMS was performed using an Agilent Mass Spectrometer with a multimode source. Analysis was performed using either a Phenomenex or a Waters C18 column and the samples were monitored at 254 nm.

### Example 1—Preparation of 3-bromo-6-(3,5-dichlorophenyl)-4H-thieno[3,2-b]pyrrole-5-carboxylic acid

### (i) Synthesis of (3,4-dibromothiophen-2-yl)(3,5-dichlorophenyl)methanone

[chemical structure]

3,4-Dibromothiophene (3.50 g, 14.5 mmol) was dissolved in dry DCM (50 mL) followed by addition of 3,5-dichlorobenzoyl chloride (4.09 g, 19.5 mmol). The reaction mixture was cooled to 0° C. before the addition of AlCl₃ (5.02 g, 37.7 mmol) in three portions. The cold bath was removed and the reaction mixture was stirred at room temperature for 3 hours before slow addition of water (25 mL) while cooling. The mixture was diluted with DCM (50 mL) and washed with 1 M NaOH aq. (2×150 mL) and Brine (2×150 mL). The organic layer was dried with MgSO₄, filtered and concentrated at reduced pressure. The residue was taken up in DCM (15 mL) and iso-hexane (5 mL) was added. The solid was filtered off and washed with iso-hexane (3 mL) to give 2.99 g white solid of the title product (50% yield). ¹H NMR (400 MHz, Chloroform-d) δ 7.70 (s, 1H), 7.66 (d, J=1.9 Hz, 2H), 7.59 (t, J=1.9 Hz, 1H). ¹³C NMR (101 MHz, CDCl₃) δ 184.92, 139.36, 136.07, 135.63, 133.03, 129.43, 128.05, 119.44, 117.38

### (ii) Synthesis of ethyl 3-bromo-6-(3,5-dichlorophenyl)-4H-thieno-[3,2-b]pyrrole-5-carboxylate

[chemical structure]

(3,4-Dibromothiophen-2-yl)(3,5-dichlorophenyl)methanone (1.00 g, 2.41 mmol) and ethyl isocyanoacetate (300 mg, 2.65 mmol) were mixed with dry THF (20 mL) and added dropwise to a suspension of NaH (120 mg, 60% in mineral oil, 3.00 mmol) in anhydrous THF (5.0 mL) over 35 minutes. The reaction mixture was stirred at room temperature for 1 hour before removal of THF under a flow of nitrogen gas. DMSO (10 mL) was added to the dark red oil followed by addition of CuI (50 mg, 0.26 mmol) and cesium carbonate (1.50 g, 4.82 mmol). The reaction mixture was stirred overnight at 80° C. under nitrogen atmosphere. The reaction mixture was acidified with 1N HCl to pH=1 and the product extracted to DCM (3×30 mL). The combined organic extracts was washed with brine (50 mL), dried over magnesium sulfate, filtrated and concentrated by rotary evaporation. The residue was recrystallized and filtrated to give 573 mg of the titled product as pale brown solid. The filtrate was purified by silica gel flash chromatography (5 to 20% EtOAc in isohexane) to give the titled product as white powder 167 mg. Total yield 73%, 740 mg. ¹H NMR (400 MHz, DMSO-d₆) δ 12.75 (br s, 1H), 7.73 (s, 1H), 7.64-7.58 (m, 3H), 4.24 (q, J=7.1 Hz, 2H), 1.23 (t, J=7.1 Hz, 3H); ¹³C NMR (101 MHz, DMSO-d₆): δ 160.89, 137.80, 135.99, 134.56, 127.88, 127.61, 126.09, 124.72, 122.80, 121.67, 94.40, 61.34, 14.10.

### Synthesis of 3-bromo-6-(3,5-dichlorophenyl)-4H-thieno[3,2-b]pyrrole-5-carboxylic acid

[chemical structure]

Ethyl 3-bromo-6-(3,5-dichlorophenyl)-4H-thieno[3,2-b]pyrrole-5-carboxylate (12 mg, 0.03 mmol) was suspended in EtOH (1.5 mL). 1M NaOH aq. (0.3 mL) was added and the mixture was allowed to stir at 80° C. for 2 hours. The mixture was acidified with 1M HCl aq. to pH 1, followed by extraction of the product to DCM (3×5 mL). The organic layers were dried with MgSO₄, filtered and concentrated by rotary evaporation. Purification by silica column chromatography (EtOAc:isohexane:HCOOH, 1:2:0.01) gave the title compound as white solid (10 mg, 89% yield). HRMS [M−H⁺]: calcd. 387.8591, found 387.8601. ¹H NMR (400 MHz, CDCl₃:MeOD, 95:5) δ 7.55 (d, J=1.9 Hz, 2H), 7.31 (t, J=1.9 Hz, 1H), 7.25 (s, 1H). ¹³C NMR (101 MHz, CDCl₃:MeOD, 95:5) δ 162.60, 137.45, 136.31, 134.65, 127.89, 127.57, 126.07, 124.78, 122.55, 110.13, 94.56.

### Example 2—Preparation of 3-cyclopropyl-6-(3,5-dichlorophenyl)-4H-thieno[3,2-b]pyrrole-5-carboxylic acid

### (i) Synthesis of ethyl 3-cyclopropyl-6-(3,5-dichlorophenyl)-4H-thieno[3,2-b]pyrrole-5-carboxylate

[chemical structure]

Ethyl 3-bromo-6-(3,5-dichlorophenyl)-4H-thieno[3,2-b]pyrrole-5-carboxylate (12 mg, 0.03 mmol), cyclopropylboronic acid (3.4 mg, 0.04 mmol), tricyclohexylphosphine (2.4 mg, 0.01 mmol), Pd(OAc)₂ (1 mg, 0.004 mmol) and K₃PO₄ (21 mg, 0.10 mmol) were dissolved in toluene (2 mL) and water (0.8 mL) and stirred at 85° C. overnight. The solvent was concentrated by rotary evaporation and the residue was purified by silica gel column chromatography (pentane:EtOAc, 20:1 to 10:1) to give the title compound as a pale yellow semi solid (6 mg, 55% yield). HRMS [M−H⁺]: calcd. 378.0117, found 378.0122; ¹H NMR (400 MHz, CDCl₃) δ 9.26 (br s, NH), 7.57 (d, J=1.9 Hz, 2H), 7.32 (t, J=1.9 Hz, 1H), 6.89 (s, 1H), 4.33 (q, J=7.1 Hz, 2H), 1.90 (m, 1H), 1.32 (t, J=7.1 Hz, 3H), 0.97 (m, 2H), 0.75 (m, 2H); ¹³C NMR (101 MHz, CDCl₃) δ 161.57, 139.91, 136.88, 134.49, 128.38, 128.09, 127.35, 125.69, 123.67, 121.86, 121.44, 61.17, 14.25, 8.70, 6.42.

### (ii) Synthesis of 3-cyclopropyl-6-(3,5-dichlorophenyl)-4H-thieno[3,2-b]pyrrole-5-carboxylic acid

[chemical structure]

Ethyl 3-cyclopropyl-6-(3,5-dichlorophenyl)-4H-thieno[3,2-b]pyrrole-5-carboxylate (14 mg, 0.04 mmol) was dissolved in THF:1M NaOH aq. (1:1, 1 mL). The mixture was stirred at 50° C. overnight and then heated to 80° C. for 4 hours. 1M HCl aq. was added until pH>1 and the product was extracted to EtOAc (3×4 mL). The combined organic layers were dried with MgSO₄, filtered and concentrated by rotary evaporation. The residue was purified by silica gel column chromatography (EtOAc:iso-hexane:HCOOH 10:90:0.05 to 30:70:0.05) followed by preparative HPLC purification (Nucleodur C18 column, 20-100% gradient of acetonitrile: H₂O with 0.05% HCOOH). The title product was isolated as white solid after freeze drying (11 mg, 82% yield). ESI-MS [M+H⁺] 352; ¹H NMR (400 MHz, CD₃CN:D₂O, 99:1) δ 10.35 (br s, NH), 7.58 (d, J=2.0 Hz, 2H), 7.32 (t, J=2.0 Hz, 1H), 6.81 (br s, 1H), 1.95 (m, 1H), 0.86 (m, 2H), 0.60 (m, 2H); ¹³C NMR (101 MHz, CD₃CN:D₂O, 99:1) δ 161.99, 140.66, 138.11, 134.69, 130.03, 128.29, 127.23, 125.36, 122.89, 122.05, 121.03, 8.99, 7.33.

### Example 3—Preparation of 3-bromo-4H-thieno[3,2-b]pyrrole-5-carboxylic acid

[chemical structure]

This compound was sourced commercially from ArkPharm (#AK27390). 1H NMR (400 MHz, DMSO-d6:D₂O, 8:2) δ 12.37 (br s, NH), 7.59 (s, 1H), 7.12 (s, 1H); ¹³C NMR (101 MHz, DMSO-d6:D2O, 8:2) δ 162.09, 139.46, 128.90, 125.82, 122.89, 107.98, 94.18.

### Example 4—Preparation of 3-Bromo-6-(4-fluorophenyl)-4H-thieno-[3,2-b]pyrrole-5-carboxylic acid

### (i)Synthesis of methyl 3-bromo-4H-thieno[3,2-b]pyrrole-5-carboxylate

[chemical structure]

4H-Thieno-[3,2-b]pyrrole-5-carboxylic acid (3.00 g, 12.2 mmol) was dissolved in DMF (10 mL) and cooled to 0° C. in an ice bath. To this solution were added KHCO3 (1.32 g, 13.2 mmol) and iodomethane (1.5 mL, 24.1 mmol). The reaction was stirred for 16 hours at room temperature before addition of water (200 mL), causing the precipitation of a light-tan solid. The precipitate was isolated via vacuum filtration and washed with water. Dried in vacuum over phosphorus pentoxide to give the titled product as white solid (2.98 g, 94% yield). ¹H NMR (400 MHz, DMSO-d6:D2O, 20:1) δ 12.55 (br s, 1H), 7.64 (s, 1H), 7.19 (br s, 1H), 3.82 (s, 3H); ¹³C NMR (101 MHz, DMSO-d6:D20, 20:1) δ 160.97, 139.80, 127.48, 126.47, 123.01, 108.35, 94.12, 51.58.

### (ii) Synthesis of methyl 3-bromo-6-iodo-4H-thieno[3,2-b]pyrrole-5-carboxylate

[chemical structure]

Methyl 3-bromo-4H-thieno[3,2-b]pyrrole-5-carboxylate (1.00 g, 3.84 mmol) was dissolved in dry DMF (2 ml) in a 10 ml vial and N-iodosuccinimide (891 mg, 3.96 mmol) was added. The reaction mixture was stirred in room temperature overnight and was then poured into water (40 mL). The precipitate formed was collected by filtration and washed with water (50 mL). The powder obtained was dissolved in EtOAc (50 mL) and washed with brine (50 mL). Organic solution was dried over MgSO₄, filtered and concentrated. The crude product was recrystallized in MeOH to give 1.13 g of the titled product as slightly grey powder (76% yield).

¹H NMR (400 MHz, DMSO-d₆) δ 12.93 (br s, 1H), 7.73 (s, 1H), 3.84 (s, 3H).

### (iii) Synthesis of methyl 3-bromo-6-(4-fluorophenyl)-4H-thieno-[3,2-b]pyrrole-5-carboxylate

[chemical structure]

A 10-20 mL microwave vial was charged with methyl 3-bromo-6-iodo-4H-thieno-[3,2-b]pyrrole-5-carboxylate (500 mg, 1.3 mmol), 4-fluorophenyl boronic acid (199 mg, 1.42 mmol), sodium carbonate (275 mg, 2.59 mmol), 1,4-dioxane (7 mL) and water (1.5 mL). Nitrogen gas was bubbled for 5 minutes through the reaction mixture before addition of bis(triphenylphosphine)palladium(II) dichloride (91 mg, 0.13 mmol). The vial was flushed with nitrogen gas, sealed and heated with microwaves to 100° C. for 2 h. The reaction mixture was poured into aqueous saturated ammonium chloride (50 mL) and the product was extracted to EtOAc (3×40 mL). The combined organic layers was washed with brine (50 mL), dried over MgSO₄, filtered and concentrated by rotary evaporation. The crude product was purified by flash silica gel column chromatography using EtOAc:petroleum ether (5:95) as eluent to give the titled product as pale yellow solid 279 mg (61% yield).

¹H NMR (400 MHz, Chloroform-d) δ 9.11 (br s, NH), 7.64 (m, 2H), 7.27 (s, 1H), 7.13 (m, 1H), 3.85 (s, 3H).

### (iv) Synthesis of 3-Bromo-6-(4-fluorophenyl)-4H-thieno-[3,2-b]pyrrole-5-carboxylic acid [General Method for Ester Hydrolysis]

[chemical structure]

Methyl 3-bromo-6-(4-fluorophenyl)-4H-thieno-[3,2-b]pyrrole-5-carboxylate (25 mg, 0.07 mmol) was suspended in water:methanol (1:1, 1 mL). 1N LiOH aq. (0.5 mL) and the reaction mixture was heated for to 95° C. for 2 hours. The reaction mixture was diluted with water (10 mL) and 1M HCl aq. was added until pH=1. The precipitate formed was extracted into EtOAc (3×15 mL) and the combined organic layers were washed with water (25 mL) and brine (25 mL). The organic layer was then dried over MgSO₄, filtered and concentrated. The residue was recrystallized from DCM to give the titled product as white solid, 10 mg (42% yield). ¹H NMR (400 MHz, DMSO-d₆) δ 12.85 (br s, 1H), 12.42 (br s, 1H), 7.67 (s, 1H), 7.64 (m, 2H), 7.27 (m, 2H).

### Example 5—Preparation of 3,6-Bis(4-fluorophenyl)-4H-thieno-[3,2-b]pyrrole-5-carboxylic acid

### (i) Synthesis of Methyl 3,6-bis(4-fluorophenyl)-4H-thieno-[3,2-b]pyrrole-5-carboxylate

[chemical structure]

A 2-5 mL microwave vial was charged with methyl 3-bromo-6-iodo-4H-thieno-[3,2-b]pyrrole-5-carboxylate (39 mg, 0.10 mmol; step (ii), Example 4), 4-fluorophenyl boronic acid (28 mg, 0.20 mmol), sodium bicarbonate (25 mg, 0.30 mmol), 1,4-dioxane (1.5 mL) and deionized water (0.5 mL). Nitrogen gas was bubbled for 10 minutes through the reaction mixture before addition of [1,1′-Bis(diphenylphosphino)ferrocene]dichloropalladium (II) (25 mg, 0.03 mmol). The vial was flushed with nitrogen gas, sealed and heated with microwaves to 120° C. for 45 minutes. The reaction mixture was poured into aqueous saturated ammonium chloride (25 mL) and the product was extracted to EtOAc (3×20 mL). The organic extracts was washed with brine (30 mL), dried over MgSO₄ and concentrated. The residue was purified by silica gel column chromatography (toluene:EtOAc, 9:1) followed by a second silica gel column (EtOAc:petroleum ether, 5:95) to give the title product as white solid, 20 mg (54% yield). ¹H NMR (400 MHz, CDCl₃) δ 9.16 (br s, NH), 7.61 (m, 2H), 7.49 (m, 2H), 7.21-6.99 (m, 4H), 3.75 (s, 3H).

### (ii) Synthesis of 3,6-Bis(4-fluorophenyl)-4H-thieno-[3,2-b]pyrrole-5-carboxylic acid

[chemical structure]

The hydrolysis was performed following the General procedure for ester hydrolysis (described in Example 4, step (iv)), starting from methyl 3,6-bis(4-fluorophenyl)-4H-thieno-[3,2-b]pyrrole-5-carboxylate (20 mg, 0.05 mmol; step (i) above) to give 10 mg of the titled product (52% yield). ESI-MS [M+H⁺] 356; ¹H NMR (400 MHz, CD₃CN) δ 10.11 (br s, NH), 7.81-7.71 (m, 4H), 7.55 (s, 1H), 7.29 (m, 2H), 7.22 (m, 2H).

### Example 6—Preparation of methyl 3-isopropenyl-6-(4-fluorophenyl)-4H-thieno-[3,2-b]pyrrole-5-carboxylate

### (i) Synthesis of methyl 3-isopropenyl-6-(4-fluorophenyl)-4H-thieno-[3,2-b]pyrrole-5-carboxylate [General method for Suzuki coupling]

[chemical structure]

A 2-5 mL microwave vial was charged with methyl 3-bromo-6-(4-fluorophenyl)-4H-thieno-[3,2-b]pyrrole-5-carboxylate (200 mg, 0.56 mmol), isopropenylboronic acid pinacol ester 4,4,5,5-tetramethyl-2-(1-methylethenyl)-1,3,2-dioxaborolane (114 mg, 0.68 mmol), sodium carbonate (120 mg, 1.13 mmol), 1,4-dioxane (3 mL) and water (0.5 mL). Nitrogen gas was bubbled through the reaction mixture for 5 minutes before addition of bis(triphenylphosphine)palladium(II) dichloride (40 mg, 0.06 mmol). The vial was flushed with nitrogen gas, capped and heated by microwaves to 100° C. for 1.5 hours. The reaction mixture was poured into aqueous saturated ammonium chloride (15 mL) and extracted into EtOAc (3×30 mL). The combined organic layers were washed with brine (50 mL), dried over magnesium sulphate, filtered and concentrated by rotary evaporation. The residue was purified by silica gel flash chromatography (EtOAc:pentane, 1:99 to 5:95). The titled product was obtained as white solid, 50 mg (28% yield). ¹H NMR (400 MHz, CDCl₃) δ 9.17 (br s, NH), 7.67 (m, 2H), 7.27 (s, 1H), 7.13 (m, 2H), 5.36 (m, 1H), 5.27 (m, 1H), 3.84 (s, 3H), 2.21 (m, 3H); ¹³C NMR (101 MHz, CDCl₃) δ 162.37 (d, J_{C-F}=246 Hz), 161.84, 137.53, 137.48, 131.24 (d, J_{C-F}=8.1 Hz), 129.47 (d, J_{C-F}=3.3 Hz), 127.79, 126.49, 126.09, 123.73, 121.28, 115.23 (d, J_{C-F}=21 Hz), 112.34, 51.81, 22.32.

### (ii) Synthesis of 3-isopropenyl-6-(4-fluorophenyl)-4H-thieno-[3,2-b]pyrrole-5-carboxylic acid

[chemical structure]

The hydrolysis was performed following the General procedure for ester hydrolysis (described in Example 4, step (iv)), starting from methyl 3-isopropenyl-6-(4-fluorophenyl)-4H-thieno-[3,2-b]pyrrole-5-carboxylate (45 mg, 0.14 mmol) to give 12 mg of the titled product as beige solid (28% yield). ESI-MS [M+H⁺] 302; ¹H NMR (400 MHz, CDCl₃) δ 9.22 (s, NH), 7.69 (m, 2H), 7.31 (s, 1H), 7.14 (m, 2H), 5.35 (m, 1H), 5.28 (m, 1H), 2.20 (m, 3H). ¹³C NMR (101 MHz, CDCl₃) δ 166.15, 162.50 (d, J_{C-F}=247 Hz), 138.37, 137.40, 131.38 (d, J_{C-F}=8.2 Hz), 129.06, 127.75, 127.13, 126.89, 125.40, 120.36, 115.49, 115.27, 112.49, 22.31.

### Example 7—Preparation of 3-isopropyl-4H-thieno[3,2-b]pyrrole-5-carboxylic acid

### (i) Synthesis of methyl 3-(prop-1-en-2-yl)-4H-thieno[3,2-b]pyrrole-5-carboxylate

[chemical structure]

Methyl 3-bromo-4H-thieno[3,2-b]pyrrole-5-carboxylate (100 mg, 0.38 mmol), bis(triphenylphosphine)palladium(II) chloride (26.99 mg, 0.04 mmol), isopropenylboronic acid pinacolester (77.5 mg, 0.46 mmol) and sodium carbonate (51 mg, 0.48 mmol) were mixed in a degassed solution of water:1,4-dioxane (1:9, 5 ml). The vial was sealed and heated by microwaves to 80° C. for 3 hours. The mixture was diluted with water (10 mL) and extracted to EtOAc (25 mL). The organic layer was dried with MgSO₄, filtered and concentrated followed by flash chromatography on aluminum oxide using ethyl acetate:iso-hexane (10:90) as eluent to give the titled product as pale yellow solid (90 mg, 68% yield). ¹H NMR (400 MHz, CDCl₃): 9.08 (br s. NH), 7.23 (s, 1H), 7.13 (d, J=1.9 Hz, 1H), 5.33 (m, 1H), 5.24 (m, 1H), 2.98 (s, 3H), 2.19 (m, 3H).

### (ii) Synthesis of methyl 3-isopropyl-4H-thieno[3,2-b]pyrrole-5-carboxylate

[chemical structure]

Methyl 3-(prop-1-en-2-yl)-4H-thieno[3,2-b]pyrrole-5-carboxylate (58 mg, 0.26 mmol) was dissolved in ethyl acetate (5 ml). Palladium on charcoal (10 mol %) was added and the mixture was hydrogenated under 8 bar for 1 hour. Purification by flash silica gel chromatography (10% ethyl acetate in iso-hexane) gave the titled product as white solid (15 mg, 26% yield). ¹H NMR (400 MHz, CDCl₃): 8.99 (brs, NH), 7.11 (d, J=1.9 Hz, 1H), 6.94 (d, J=1.0 Hz, 1H), 3.90 (s, 3H), 3.08 (heptd, J=6.9, 1.0 Hz, 1H), 1.35 (d, J=6.9 Hz, 6H).

### (iii) Synthesis of 3-isopropyl-4H-thieno[3,2-b]pyrrole-5-carboxylic acid

[chemical structure]

Methyl 3-isopropyl-4H-thieno[3,2-b]pyrrole-5-carboxylate (15 mg, 0.07 mmol) was dissolved in 1M LiOH in water (0.5 mL) and methanol (0.5 mL). The mixture was heated to 100° C. for 2 h. The reaction mixture was cooled and diluted with water (5 mL). Acidification until pH=1 and extraction with ethyl acetate (2×15 mL) followed by purification by silica gel flash chromatography (20% ethyl acetate and 1% formic acid in iso-hexane) gave desired product as white solid (14 mg, 56% yield). ESI-MS [M+H⁺] 210; ¹H NMR (400 MHz, CDCl₃): 9.11 (brs, NH), 7.27 (d, J=1.8 Hz, 1H), 7.01 (d, J=1.0 Hz, 1H), 3.10 (heptd, J=6.9, 1.0 Hz, 1H), 1.37 (d, J=5.9 Hz, 6H).

### Example 8—Preparation of 3-cyclopropyl-4H-thieno[3,2-b]pyrrole-5-carboxylic acid

### (i) Synthesis of methyl 3-cyclopropyl-4H-thieno[3,2-b]pyrrole-5-carboxylate

[chemical structure]

Methyl 3-bromo-4H-thieno[3,2-b]pyrrole-5-carboxylate (200 mg, 0.77 mmol), cyclopropylboronic acid (88 mg, 1.0 mmol), palladium acetate (26 mg, 0.12 mmol), tricyclohexylphosphine (65 mg, 0.23 mmol) and potassium phosphate (571 mg, 2.7 mmol) were mixed in a degassed solution of toluene (16 mL) and water (0.8 mL). The mixture was stirred at 85° C. overnight in a sealed glass vial. After cooling, aqueous saturated sodium bicarbonate (20 mL) was added and the crude product was extracted to ethyl acetate (2×30 mL). Purification by silica gel flash chromatography (10% ethyl acetate in petroleum ether) gave the titled product as pale yellow solid (98 mg, 58% yield). ¹H NMR (CDCl3): 9.05 (br s, NH), 7.09 (m, 1H), 6.86 (m, 1H), 3.9 (s, 3H), 1.87 (m, 1H), 0.96-0.90 (m, 2H), 0.75-0.70 (m, 2H).

### (ii) Synthesis of 3-cyclopropyl-4H-thieno[3,2-b]pyrrole-5-carboxylic acid

[chemical structure]

Methyl 3-cyclopropyl-4H-thieno[3,2-b]pyrrole-5-carboxylate (50 mg, 0.23 mmol) was dissolved in methanol:1M Lithium hydroxide (aq) (1:1, 1 mL). The mixture was heated to 100° C. for 1 hour. Acidification (until pH=1) and extraction to ethyl acetate (2×20 mL) followed by silica gel flash chromatography (iso-hexane:ethyl acetate, 5:1, containing 0.5% formic acid) gave the titled product as white solid (15 mg, 31% yield). ESI-MS [M+H⁺] 208; ¹H NMR (DMSO-d₆): 12.51 (br s, 1H), 12.11 (br s, 1H), 6.89 (d, J=1.9, 1H), 6.87 (d, J=0.8, 1H), 2.18 (dm, J=0.8, 1H), 0.92-0.85 (m, 2H), 0.69-0.64 (m, 2H).

### Example 9—Preparation of 6-(3-acetamidophenyl)-3-bromo-4H-thieno[3,2-b]pyrrole-5-carboxylic acid

### (i) Synthesis of methyl 6-(3-acetamidophenyl)-3-bromo-4H-thieno[3,2-b]pyrrole-5-carboxylate

[chemical structure]

Methyl 3-bromo-6-iodo-4H-thieno[3,2-b]pyrrole-5-carboxylate (173 mg, 0.45 mmol), 3-acetamido-phenylboronic acid (80 mg, 0.45 mmol), tetrakis triphenylphosphine palladium (0) (26 mg, 0.02 mmol) and potassium carbonate (0.45 ml, 2M aq. solution, 0.90 mmol) were mixed under nitrogen atmosphere in degassed 1,2-dimethoxyethane (4 mL). The reaction mixture was refluxed overnight. Water (10 mL) was added and the mixture was extracted with ethyl acetate (2×15 mL). After drying with MgSO₄, filtering and evaporation of the solvent, the product was purified by silica gel flash chromatography (40% ethyl acetate in iso-hexane) to give the desired product (62 mg, 32% yield). ESI-MS [M+H⁺] 395, 393; ¹H NMR (400 MHz, CDCl₃) δ 9.05 (br s, 1H), 7.75 (m, 1H), 7.48 (dm, J=7.7 Hz, 1H), 7.39-7.29 (m, 2H), 7.20 (br s, 1H), 7.14 (br s, 1H), 3.80 (s, 3H), 2.13 (s, 3H).

### (ii) Synthesis of 6-(3-Acetamidophenyl)-3-bromo-4H-thieno[3,2-b]pyrrole-5-carboxylic acid

[chemical structure]

Methyl 6-(3-acetamidophenyl)-3-bromo-4H-thieno[3,2-b]pyrrole-5-carboxylate (58 mg, 0.15 mmol) was dissolved in methanol (0.5 mL) and 1M Lithium hydroxide (aq) (0.5 mL). The mixture was heated to 100° C. for 4 hours. Water (10 mL) was added and the mixture was acidified to pH=1. The mixture was extracted with ethyl acetate (2×25 mL) and the combined organic layers was concentrated under reduce pressure. The residue was purified by silica gel flash chromatography (iso-hexane:ethyl acetate, 1:2, containing 0.5% formic acid) to give the crude product. The crude material was taken up in ethyl acetate and heated to reflux. Iso-hexane was added until the product precipitated. The mixture was kept in a cooler overnight and then filtrated to give the titled product (12 mg, 21% yield). ESI-MS [M+H⁺] 379, 381; ¹H NMR (400 MHz, DMSO-d₆) δ 12.71 (br s, COOH), 12.39 (br s, NH), 10.00 (br s, NH), 7.81 (m, 1H), 7.66 (s, 1H), 7.59 (dm, J=8.2 Hz, 1H), 7.33 (dd, J=8.2, 7.7 Hz, 1H), 7.26 (dm, J=7.7, Hz, 1H), 2.06 (s, 3H).

### Example 10—Preparation of 3-bromo-6-(3-((tert-butoxycarbonyl)amino)phenyl)-4H-thieno[3,2-b]pyrrole-5-carboxylic acid

### (i) Synthesis of methyl 3-bromo-6-(3-((tert-butoxycarbonyl)amino)phenyl)-4H-thieno[3,2-b]pyrrole-5-carboxylate

[chemical structure]

Methyl 3-bromo-6-iodo-4H-thieno[3,2-b]pyrrole-5-carboxylate (170 mg, 0.44 mmol), 3-(N-Boc-amino)phenylboronic acid (115 mg, 0.48 mmol), tetrakis triphenylphosphine palladium (0) (25 mg, 0.02 mmol) and potassium carbonate (0.66 ml, 2M aq solution, 1.32 mmol) were mixed under nitrogen atmosphere in degassed 1,2-dimethoxyethane (4 mL). The reaction mixture was refluxed overnight. Water (10 mL) was added and the mixture was extracted with ethyl acetate (2×15 mL). After drying with MgSO₄, filtering and evaporation of the solvent, the product was purified by silica gel flash chromatography (20% ethyl acetate in iso-hexane) to give the desired product (199 mg, 49% yield). ESI-MS [M+H⁺] 451, 453; ¹H NMR (400 MHz, CDCl₃) δ 9.10 (br s, 1H), 7.63 (br s, 1H), 7.45 (dm, J=7.7 Hz, 1H), 7.36 (dd, J=7.7, 7.6 Hz, 1H), 7.32 (dm, J=7.6 Hz, 1H), 7.25 (m, 1H), 6.52 (br s, 1H), 3.86 (s, 3H), 1.52 (s, 9H).

### (ii) Synthesis of 3-bromo-6-(3-((tert-butoxycarbonyl)amino)phenyl)-4H-thieno[3,2-b]pyrrole-5-carboxylic acid

[chemical structure]

Methyl 3-bromo-6-(3-((tert-butoxycarbonyl)amino)phenyl)-4H-thieno[3,2-b]pyrrole-5-carboxylate (58 mg, 0.13 mmol) was dissolved in methanol:1M Lithium hydroxide (aq) (1:1, 1 mL). The mixture was heated to 100° C. for 1 hour. Acidification (until pH=1) and extraction to ethyl acetate (2×20 mL) followed by silica gel flash chromatography (30% ethyl acetate, 0.5% formic acid in iso-hexane) to give the titled compound as white solid (50 mg, 89% yield). ESI-MS [M+H⁺] 437, 439; ¹H NMR (400 MHz, DMSO-d₆) δ 12.72 (br s, 1H), 12.36 (br s, 1H), 9.40 (br s, 1H), 7.68 (m, 1H), 7.64 (s, 1H), 7.43 (dm, J=8.3 Hz, 1H), 7.28 (dd, J=8.3, 7.7 Hz, 1H), 7.17 (dm, J=7.7, Hz, 1H), 1.48 (s, 9H).

### Example 11—Preparation of 6-(3-aminophenyl)-3-bromo-4H-thieno[3,2-b]pyrrole-5-carboxylic acid hydrochloride

[chemical structure]

3-bromo-6-(3-((tert-butoxycarbonyl)amino)phenyl)-4H-thieno[3,2-b]pyrrole-5-carboxylic acid (20 mg, 0.05 mmol) was treated with 4M HCl in dioxane (1 mL). After 3 hours stirring in room temperature the solvent was removed by rotary evaporation and the residue dried in vacuum to give the titled product as pale yellow solid (17 mg, quantitative yield). ESI-MS [M+H⁺] 337, 339; ¹H NMR (400 MHz, CD₃OD) δ 7.80-7.73 (m, 2H), 7.60 (dm, J=8.1 Hz, 1H), 7.45 (s, 1H), 7.35 (dm, J=8.1 Hz, 1H).

### Example 12—Preparation of 3-isopropyl-6-(4-fluorophenyl)-4H-thieno-[3,2-b]pyrrole-5-carboxylic acid

### (i) Synthesis of methyl 3-isopropyl-6-(4-fluorophenyl)-4H-thieno-[3,2-b]pyrrole-5-carboxylate

[chemical structure]

Methyl 3-isopropenyl-6-(4-fluorophenyl)-4H-thieno-[3,2-b]pyrrole-5-carboxylate (70 mg, 0.22 mmol) was dissolved in ethyl acetate (4 mL), palladium on carbon (10%, 15 mg) was added and the reaction flask was placed in a hydrogenation autoclave, flushed with hydrogen and stirred in ambient temperature under 7 bar hydrogen atmosphere for 3 hours. The reaction mixture was filtered through a plug of celite, the filtrate was concentrated and the residue was recrystallized from ethyl acetate to give the titled compound as pale yellow solid (40 mg, 57% yield). ESI-MS [M+H⁺] 318; ¹H NMR (400 MHz, CDCl₃) δ 9.02 (br s, NH), 7.66 (m, 2H), 7.12 (m, 2H), 6.99 (d, J=1.0 Hz, 1H), 3.83 (s, 3H), 3.10 (heptd, J=6.9, 1.0 Hz, 1H), 1.38 (d, J=6.9 Hz, 6H).

### (ii) Synthesis of 3-isopropyl-6-(4-fluorophenyl)-4H-thieno-[3,2-b]pyrrole-5-carboxylic acid

[chemical structure]

The hydrolysis was performed following the General procedure for ester hydrolysis (described in Example 4, step (iv)), starting from methyl 3-isopropyl-6-(4-fluorophenyl)-4H-thieno-[3,2-b]pyrrole-5-carboxylate (58 mg, 0.18 mmol) to give 40 mg of the titled product (72% yield). ESI-MS [M+H⁺] 304; ¹H NMR (400 MHz, CDCl₃) δ 9.15 (br s, 1H), 7.70 (m, 2H), 7.12 (m, 2H), 7.04 (d, J=1.0 Hz, 1H), 3.10 (heptd, J=6.9, 1.0 Hz, 1H), 1.37 (d, J=6.9 Hz, 6H); ¹³C NMR (101 MHz, CDCl₃) δ 166.39, 162.36 (d, J_{C-F}=247 Hz), 139.89, 133.17, 131.35 (d, J_{C-F}=8.2 Hz), 129.34 (d, J_{C-F}=3.3 Hz), 126.79, 125.66, 124.16, 119.96, 115.29 (d, J_{C-F}=22 Hz), 28.32, 22.54.

### Example 13—Preparation of 3-cyclopropyl-6-(4-fluorophenyl)-4H-thieno-[3,2-b]pyrrole-5-carboxylic acid

### (i) Synthesis of methyl 3-cyclopropyl-6-(4-fluorophenyl)-4H-thieno-[3,2-b]pyrrole-5-carboxylate

[chemical structure]

A 10-20 mL microwave vial was charged with methyl 3-bromo-6-(4-fluorophenyl)-4H-thieno-[3,2-b]pyrrole-5-carboxylate (100 mg, 0.28 mmol), cyclopropylboronic acid (29 mg, 0.34 mmol), toluene (5 mL), potassium phosphate (210 mg, 0.99 mmol) and water (0.3 mL). Nitrogen gas was bubbled through the reaction mixture for 5 min before addition of Pd(OAc)₂ (10 mg, 0.04 mmol) and Cy₃P (24 mg, 0.08 mmol). The vial was sealed and heated overnight at 90° C. The reaction mixture was poured into saturated aqueous ammonium chloride (30 mL) and extracted to ethyl acetate (3×40 mL). The combined organic layers were washed with brine (50 mL), dried over magnesium sulphate, filtered and concentrated under reduced pressure. The crude product was purified by silica gel column chromatography (5% to 10% EtOAc in petroleum ether) to give 46 mg of the titled product (52% yield). ESI-MS [M+H⁺] 316; ¹H NMR (400 MHz, CD₃CN) δ 10.32 (br s, 1H), 7.72 (m, 2H), 7.20 (m, 2H), 6.93 (d, J=1.0 Hz, 1H), 3.82 (s, 3H), 2.07 (dm, J=1.0 Hz, 1H), 1.03-0.95 (m, 2H), 0.77-0.70 (m, 2H).

### (ii) Synthesis of 3-cyclopropyl-6-(4-fluorophenyl)-4H-thieno-[3,2-b]pyrrole-5-carboxylic acid

[chemical structure]

The hydrolysis was performed following the General procedure for ester hydrolysis (described in Example 4, step (iv)), starting from 3-cyclopropyl-6-(4-fluorophenyl)-4H-thieno-[3,2-b]pyrrole-5-carboxylate (40 mg, 0.13 mmol) to give 23 mg of the titled product (60% yield). ESI-MS [M+H⁺] 302; ¹H NMR (400 MHz, CDCl₃) δ 9.19 (br s, 1H), 7.69 (m, 2H), 7.12 (m, 2H), 6.95 (d, J=1.1 Hz, 1H), 1.89 (m, 1H), 1.03-0.91 (m, 2H), 0.82-0.66 (m, 2H).

### Example 14—Preparation of 3-bromo-6-(4-sulfamoylphenyl)-4H-thieno-[3,2-b]pyrrole-5-carboxylic acid

### (i) Synthesis of methyl 3-bromo-6-(4-sulfamoylphenyl)-4H-thieno-[3,2-b]pyrrole-5-carboxylate

[chemical structure]

A 2-5 mL microwave vial was charged with methyl 3-bromo-6-iodo-4H-thieno-[3,2-b]pyrrole-5-carboxylate (200 mg, 0.52 mmol), 4-sulfamoylphenyl boronic acid (115 mg, 0.57 mmol), potassium carbonate (143 mg, 1.04 mmol), toluene (2.0 mL), ethanol (1.0 mL) and water (1.0 mL). Nitrogen gas was bubbled through the reaction mixture for 5 minutes before addition of tetrakis(triphenylphosphine)palladium(0) (30 mg, 0.03 mmol). The vial was flushed with nitrogen gas, capped and heated by microwaves to 95° C. for 2.5 hours. The reaction mixture was poured into aqueous saturated ammonium chloride (15 mL) and extracted into EtOAc (3×30 mL). The combined organic layers were washed with brine (50 mL), dried over magnesium sulphate, filtered and concentrated by rotary evaporation. The residue was purified by silica gel chromatography (3% methanol in DCM) to give the titled product pale brown solid, 103 mg (48% yield). ESI-MS [M−H⁺] 413, 415; ¹H NMR (400 MHz, CD₃CN) δ 10.52 (br s, 1H), 7.94 (m, 2H), 7.83 (m, 2H), 7.44 (s, 1H), 5.73 (br s, 2H), 3.81 (s, 3H).

### (ii) Synthesis of 3-bromo-6-(4-sulfamoylphenyl)-4H-thieno-[3,2-b]pyrrole-5-carboxylic acid

[chemical structure]

The hydrolysis was performed following the General procedure for ester hydrolysis (described in Example 4, step (iv)), starting from methyl 3-cyclopropyl-6-(4-fluorophenyl)-4H-thieno-[3,2-b]pyrrole-5-carboxylate (25 mg, 0.06 mmol) to give 12 mg of the titled product (50% yield). ESI-MS [M+H⁺] 401, 403; ¹H NMR (400 MHz, CD₃CN:D₂O, 9:1) δ 10.46 (br s, 1H), 7.94 (m, 2H), 7.87 (m, 2H), 7.48 (s, 1H), 5.74 (br s, 2H).

### Example 15—Preparation of 3-cyano-6-(4-fluorophenyl)-4H-thieno-[3,2-b]pyrrole-5-carboxylic acid

### (i) Synthesis of methyl 3-cyano-4H-thieno-[3,2-b]pyrrole-5-carboxylate

[chemical structure]

Methyl 4H-thieno[3,2-b]pyrrole-5-carboxylate (200 mg, 0.77 mmol) and copper cyanide (138 mg, 1.54 mmol) was dissolved in DMF (3 mL) and heated to 155° C. for 5 hours. The reaction mixture was cooled to room temperature and a solution of FeCl₃ (7.0 g, 43 mmol) in water (20 mL) with 1N HCl (2 mL, 2 mmol) was added. The reaction mixture was heated to 75° C. for 30 min and then extracted with EtOAc (2×30 mL). The combined organic extracts were washed with water (40 mL) and brine (40 mL), dried over anhydrous magnesium sulphate, filtrated and the solvent was removed in vacuum. The crude product was purified by silica column chromatography (ethyl acetate:hexane, 1:4) to give the titled compound as white solid (135 mg, 85% yield). ESI-MS [M−H⁺] 205; ¹H NMR (400 MHz, CD₃CN) δ 10.76 (br s, 1H), 8.19 (s, 1H), 7.18 (d, J=1.6 Hz, 1H), 3.89 (s, 3H).

### (ii) Synthesis of methyl 3-cyano-6-iodo-4H-thieno-[3,2-b]pyrrole-5-carboxylate

[chemical structure]

The reaction was performed following the same procedure as for Methyl 3-bromo-6-iodo-4H-thieno[3,2-b]pyrrole-5-carboxylate, starting form methyl 3-cyano-4H-thieno-[3,2-b]pyrrole-5-carboxylate (135 mg, 0.65 mmol) to give the titled product as light grey solid (103 mg, yield 48%). ESI-MS [M+H⁺] 333; ¹H NMR (400 MHz, (CD₃)₂SO) δ 13.29 (br s, 1H), 8.64 (s, 1H), 3.86 (s, 3H). ¹³C NMR (101 MHz, (CD₃)₂SO) δ 160.44, 141.54, 137.85, 132.11, 128.29, 118.53, 114.08, 96.45, 52.19.

### (iii) Synthesis of methyl 3-cyano-6-(4-fluorophenyl)-4H-thieno-[3,2-b]pyrrole-5-carboxylate

[chemical structure]

The reaction was performed following the same procedure as for methyl 3-bromo-6-(4-sulfamoylphenyl)-4H-thieno-[3,2-b]pyrrole-5-carboxylate. Starting form methyl 3-cyano-6-iodo-4H-thieno-[3,2-b]pyrrole-5-carboxylate (150 mg, 0.45 mmol) and 4-fluorophenylboronic acid (70 mg, 0.50 mmol) with heating to 100° C. for 1.5 hours. Final recrystallization from acetonitrile gave the titled product as white solid (87 mg, 64% yield). ESI-MS [M+H⁺] 301; ¹H NMR (400 MHz, (CD₃)₂SO) δ 13.08 (br s, 1H), 8.65 (s, 1H), 7.65 (m, 2H), 7.30 (m, 1H), 3.78 (s, 3H).

### (iv) Synthesis of 3-cyano-6-(4-fluorophenyl)-4H-thieno-[3,2-b]pyrrole-5-carboxylic acid

[chemical structure]

Methyl 3-cyano-6-(4-fluorophenyl)-4H-thieno-[3,2-b]pyrrole-5-carboxylate (40 mg, 0.13 mmol) was mixed with water (2.0 mL), 1,4-dioxane (4 mL) and 1N LiOH aq. solution (0.13 ml, 0.13 mmol). The reaction mixture was heated for 4 hours at 60° C. Additional 1N LiOH aq. solution (0.13 ml, 0.13 mmol) and the reaction mixture was heated for 30 hours at 70° C. Water (5.0 mL) was added and the mixture was acidified with 1M HCl aq. solution to pH 1. The precipitate formed was collected by filtration and purified by preparative HPLC (Nucleodur C18 column, 50-100% gradient of acetonitrile: H₂O with 0.1% TFA) to give the titled product as white solid after freeze drying (12 mg, 31% yield). ESI-MS [M−H⁺] 285; ¹H NMR (400 MHz, CD₃CN) δ 10.80 (br s, 1H), 9.98 (br s, 1H), 8.23 (s, 1H), 7.71 (m, 2H), 7.22 (m, 2H); ¹³C NMR (101 MHz, CD₃CN) δ 162.76 (d, J_{C-F}=245 Hz), 161.60, 140.88, 137.24, 131.85 (d, J_{C-F}=8.2 Hz), 129.80 (br s), 126.32, 123.96, 123.17, 115.58 (d, J_{C-F}=22 Hz), 113.91, 96.65.

### Example 16—Preparation of 6-(4-fluorophenyl)-4H-thieno-[3,2-b]pyrrole-3,5-dicarboxylic acid

[chemical structure]

The hydrolysis was performed following the General procedure for ester hydrolysis (described in Example 4, step (iv)), starting from methyl 3-cyano-6-(4-fluorophenyl)-4H-thieno-[3,2-b]pyrrole-5-carboxylate (40 mg, 0.13 mmol) to give 26 mg of the titled product (68% yield). ESI-MS [M+H⁺] 305; ¹H NMR (400 MHz, DMSO-d₆) δ 13.03 (br s, 1H), 10.78 (br s, 1H), 8.34 (s, 1H), 7.99 (br s, 1H), 7.68 (m, 2H), 7.29 (m, 2H); ¹³C NMR (101 MHz, DMSO-d₆) δ 163.19, 162.33, 161.77 (d, J_{C-F}=245 Hz), 137.54, 137.23, 131.55 (d, J_{C-F}=8.2 Hz), 130.13 (br s), 125.34, 123.52, 122.04, 119.66, 115.49 (d, J_{C-F}=22 Hz).

### Example 17—Preparation of 4-(3,5-dichlorophenyl)-6H-thieno[2,3-b]pyrrole-5-carboxylic acid

### (i) Synthesis of methyl 4-iodo-6H-thieno[2,3-b]pyrrole-5-carboxylate

[chemical structure]

Commercially available Methyl 6H-thieno[2,3-b]pyrrole-5-carboxylate (500 mg, 2.7 mmol) was dissolved in dry DMF (10 mL) and cooled on an ice-bath. A solution of N-Iodosuccinimide (683 mg, 3.04 mmol) in DMF (3.0 mL) was added dropwise over a period of 15 minutes. The mixture was then allowed to reach room temperature overnight. Water (40 mL) was added and the precipitate was filtered off. The solid was recrystallized from methanol-water and filtered. The filtrate was purified by preparative HPLC (Nucleodur C18 column, 40-80% gradient of acetonitrile in water with 0.1% TFA) to give the titled compound after freeze drying as white solid (50 mg, 6% yield). ESI-MS [M−H⁺] 306; ¹H NMR (400 MHz, CDCl₃) δ 9.63 (br s, NH) 6.97 (d, J=5.5 Hz, 1H), 6.9 (d, J=5.5 Hz, 1H), 3.94 (s, 3H).

### (ii) Synthesis of methyl 4-(3,5-dichlorophenyl)-6H-thieno[2,3-b]pyrrole-5-carboxylate

[chemical structure]

Methyl 4-iodo-6H-thieno[2,3-b]pyrrole-5-carboxylate (50 mg, 0.16 mmol) and 3,5-dichlorophenylboronic acid (37 mg, 0.19 mmol) were dissolved in a deoxygenated water:1,4-dioxane (1:9, 1.5 mL). Bis(triphenylphosphine)palladium(II) dichloride (5.7 mg, 0.01 mmol) and sodium carbonate (36 mg, 0.34 mmol) was then added. The vial was capped and heated to 90° C. overnight. The mixture was diluted with water (15 mL) and extracted with ethyl acetate (2×15 mL). The combined organic layers were dried with MgSO₄, filtered and concentrated by rotary evaporation. The residue was purified by silica gel flash chromatography (20% ethyl acetate in iso-hexane) to give the titled compounds as pale yellow solid, (35 mg, 66% yield). ESI-MS [M−H⁺] 324, 326; ¹H NMR (400 MHz, CDCl₃) δ 9.31 (br s, NH), 7.51 (d, J=1.9 Hz, 2H), 7.34 (t, J=1.9 Hz, 1H), 7.02-6.97 (m, 2H), 3.84 (s, 3H).

### (iii) Synthesis of 4-(3,5-dichlorophenyl)-6H-thieno[2,3-b]pyrrole-5-carboxylic acid

[chemical structure]

Methyl 4-(3,5-dichlorophenyl)-6H-thieno[2,3-b]pyrrole-5-carboxylate (30 mg, 0.09 mmol) was hydrolyzed in a 50:50 mixture of 1 M LiOH in water and methanol (1 mL) at 100° C. for 4 hours. The mixture was acidified to pH>1 and extracted with ethyl acetate (2×15 mL). The combined organic layers were dried with MgSO₄, filtered and concentrated by rotary evaporation. The residue was purified by silica gel flash chromatography followed by flash chromatography (30% ethyl acetate and 0.5% formic acid in iso-hexane) to give the titled compound as white solid (8 mg, 27% yield). ESI-MS [M−H⁺] 310, 312; ¹H NMR (400 MHz, DMSO-d₆) δ 12.74 (br s, 1H), 12.31 (br s, 1H), 7.58 (d, J=1.9 Hz, 2H), 7.54 (t, J=1.9 Hz, 1H), 7.20 (d, J=5.4 Hz, 1H), 7.01 (d, J=5.4 Hz, 1H).

### Example 18—Preparation of 6-(3-chloro-4-((methylsulfonyl)methyl)phenyl)-3-cyclopropyl-4H-thieno[3,2-b]pyrrole-5-carboxylic acid

### (i) Synthesis of methyl 3-bromo-6-(3-chloro-4-((methylsulfonyl)methyl)phenyl)-4H-thieno[3,2-b]pyrrole-5-carboxylate (A) and methyl 3,6-bis(3-chloro-4-((methylsulfonyl)methyl)phenyl)-4H-thieno[3,2-b]pyrrole-5-carboxylate (B)

[chemical structure]

Methyl 3-bromo-6-iodo-4H-thieno[3,2-b]pyrrole-5-carboxylate (375 mg, 0.971 mmol), 2-(3-chloro-4-((methylsulfonyl)methyl)phenyl)-4,4,5,5-tetramethyl-1,3,2-dioxaborolane (385 mg, 1.17 mmol), Pd(dppf)Cl₂-DCM, (34.1 mg, 0.04 mmol) and sodium carbonate (412 mg, 3.9 mmol) were mixed in deoxygenated dioxane (10 ml). The mixture was then heated in a sealed tub at 100° C. for 4 hours. Flash chromatography (SiO₂), isohexane-ethyl acetate 2:1 gave 140 mg of compound A. Compound B was eluted using isohexane-ethyl acetate 1:2 followed by prep-HPLC (30-70% MeCN in water, 0.1% TFA) C18 column. Yield: 40 mg. ¹H NMR B (CDCl₃): 9.3 (br s, 1H), 7.63 (m, 6H), 7.42 (s, 1H), 4.48 (d, J=4.6 Hz, 4H), 3.82 (s, 3H), 2.84 (s, 6H), 1.98 (s, 2H).

### (ii) Synthesis of methyl 6-(3-chloro-4-((methylsulfonyl)methyl)phenyl)-3-cyclopropyl-4H-thieno[3,2-b]pyrrole-5-carboxylate (A) and methyl 6-(3-chloro-4-((methylsulfonyl)methyl)phenyl)-4H-thieno[3,2-b]pyrrole-5-carboxylate (B)

[chemical structure]

Cyclopropyl boronic acid (250 mg, 1.47 mmol), methyl 3-bromo-6-(3-chloro-4-((methylsulfonyl)methyl)phenyl)-4H-thieno[3,2-b]pyrrole-5-carboxylate (130 mg, 0.28 mmol), Pd(dppf)Cl₂-DCM (23 mg, 0.028 mmol) and potassium carbonate (245 mg, 1.77 mmol) were dissolved in 10 ml of degassed dioxane-water and heated over night at 95° C. Extraction with ethyl acetate followed by preparative HPLC, 40-80% MeCN, 15 min gradient, flow=15 ml/min, C18 column gave the desired compound A (25 mg, 23%): ¹H NMR (CDCl₃): 9.25 (s, 1H), 7.8 (d, J=1.74, 1H), 7.62 (dd, J=8.06 and 1.78 Hz, 1H), 7.57 (d J=8.05, 1H), 7.33 (d, J=5.33, 1H), 6.93 (d, J=5.35, 1H), 4.46 (s, 2H), 3.79 (s, 3H), 3.05 (d, J=0.88, 3H), 1.94 (s, 1H) and compound B (30 mg, 30%): ¹H NMR (CDCl₃): 9.27 (s, 1H), 7.84 (d, J=1.68, 1H), 7.65 (m, 2H), 6.91 (d, J=1.08 Hz, 1H), 4.52 (s, 2H), 3.87 (s, 3H), 2.88 (s, 3H), 1.9 (m, 1H), 0.97 (m, 2H), 0.75 (m, 2H).

### (iii) Synthesis of 6-(3-chloro-4-((methylsulfonyl)methyl)phenyl)-3-cyclopropyl-4H-thieno[3,2-b]pyrrole-5-carboxylic acid

[chemical structure]

Methyl 6-(3-chloro-4-((methylsulfonyl)methyl)phenyl)-4H-thieno[3,2-b]pyrrole-5-carboxylate (30 mg, 0.07 mmol) was taken up in methanol and 1M lithium hydroxide and heated at 100° C. for 4 h. Water was added and the mixture acidified with 1M hydrochloric acid. Extraction with ethyl acetate followed by flash chromatography (1:1 ethyl acetate-iso hexane+0.5% formic acid) gave the desired product (22 mg, 75%). ¹H NMR (CD₃OD): 7.76 (d, J=1.77 Hz, 1H), 7.59 (dd, J=8.05 and 1.79, 1H), 7.49 (d, J=8.06 Hz, 1H), 6.77 (d, J=0.93 Hz, 1H), 4.56 (s, 1H), 2.87 (s, 3H), 1.97 (m, 1H), 0.87 (m, 2H), 0.62 (m, 2H).

### Example 19—Preparation of 3,6-bis(3-chloro-4-((methylsulfonyl)methyl)phenyl)-4H-thieno[3,2-b]pyrrole-5-carboxylic acid

### (i) Synthesis of 3,6-bis(3-chloro-4-((methylsulfonyl)methyl)phenyl)-4H-thieno[3,2-b]pyrrole-5-carboxylic acid

[chemical structure]

Methyl 3,6-bis(3-chloro-4-((methylsulfonyl)methyl)phenyl)-4H-thieno[3,2-b]pyrrole-5-carboxylate was dissolved in methanol and 1 M LiOH and heated to 100° C. for 2-4 h. Water (25 ml was added and the pH was set to 7. The solution was extracted with ethyl acetate and purified by flash chromatography (silica) using ethyl acetate-iso-hexane; 2:1, +0.5% formic acid to give the desired product (10 mg, 34%). ¹H NMR (MeOD): 7.87 (dd, J=6.9 and 1.8 Hz, 1H), 7.7 (m, 3H), 4.69 (d, J=6.5, 4H), 2.99 (d, J=2.76, 6H).

### Example 20—Preparation of 6-(3-chloro-4-((methylsulfonyl)methyl)phenyl)-4H-thieno[3,2-b]pyrrole-5-carboxylic acid

### (i) 6-(3-chloro-4-((methylsulfonyl)methyl)phenyl)-4H-thieno[3,2-b]pyrrole-5-carboxylic acid [General Method B for Ester Hydrolysis]

[chemical structure]

Methyl 6-(3-chloro-4-((methylsulfonyl)methyl)phenyl)-3-cyclopropyl-4H-thieno[3,2-b]pyrrole-5-carboxylate (25 mg, 0.065 mmol) was taken up in methanol and 1M lithium hydroxide and heated at 100° C. for 4 h. Water was added and the mixture acidified with 1M hydrochloric acid. Extraction with ethyl acetate followed by flash chromatography (1:1 ethyl acetate-iso hexane+0.5% formic acid gave the desired product (18 mg, 74%). ¹H NMR (CD₃OD): 7.89 (d, J=1.74, 1H), 7.72 (dd, J=8 and 1.8, 1H), 7.59 d, J=8, 1H), 7.43 (d, J=5.34, 1H), 7.03 (d, J=5.33, 1H), 4.65 (s, 2H), 2.97 (s, 3H).

### Example 21—Preparation of 3-acetyl-6-(3,5-dichlorophenyl)-4H-thieno[3,2-b]pyrrole-5-carboxylic acid

### (i) Synthesis of methyl 3-acetyl-4H-thieno[3,2-b]pyrrole-5-carboxylate

[chemical structure]

Methyl 3-bromo-4H-thieno[3,2-b]pyrrole-5-carboxylate (100 mg, 0.384 mmol), tributyl(1-ethoxyvinyl)stannane (208 mg, 0.577 mmol) and tetrakis triphenylphosphinepalladium(0) (44 mg, 0.0384 mmol) were mixed in DMF (1.5 ml) and heated overnight at 100° C. Extraction with ethyl acetate. Silica filtration, then flash (silica) on Biotage isolera (5-40% ethyl acetate in iso-hexane). The vinylic product was hydrolysed during work-up to the desired compound (34 mg, 39%). ¹H NMR (CDCl₃): 9.87 (s, 1H), 8.02 (s, 1H), 7.13 (d, J=2.05, 1H), 3.91 (s, 3H), 2.58 (s, 3H).

### (ii) Synthesis of methyl 3-acetyl-6-iodo-4H-thieno[3,2-b]pyrrole-5-carboxylate

[chemical structure]

Methyl 3-acetyl-4H-thieno[3,2-b]pyrrole-5-carboxylate (35 mg, 0.152 mmol) was dissolved in DMF (1 ml). N-Iodosuccinimide (38 mg, 0.168 mmol) was added and the mixture were left over-night. One equiv. N-Iodosuccinimide was added to complete the reaction. Water was added and the product was extracted with ethyl acetate and purified by flash chromatography (SiO₂) (5-40% ethyl acetate in iso-hexane) to give the target compound (35 mg, 65%). ¹H NMR (CDCl₃): 9.99 (s, NH), 8.05 (s, 1H), 3.95 (s, 3H), 2.57 (s, 3H).

### (iii) Synthesis of methyl 3-acetyl-6-(3,5-dichlorophenyl)-4H-thieno[3,2-b]pyrrole-5-carboxylate [General Method B for Suzuki Coupling]

[chemical structure]

Methyl 3-acetyl-6-iodo-4H-thieno[3,2-b]pyrrole-5-carboxylate (35 mg, 0.1 mmol), 3,5-dichlorophenyl boronic acid (26 mg, 0.14 mmol), PdCl₂(PPh₃) (8.57 mg, 0.0122 mmol) and sodium carbonate (30 mg, 0.283 mmol) were mixed in deoxygenated dioxane (2 ml, 10% water). The mixture was heated at 90° C. over-night. Water was added and the crude mixture was extracted with ethyl acetate. Purification by flashchromatograpy (5-40% ethyl acetate in isohexane) gave the target compound (11 mg, 30%). ¹H NMR (CDCl₃): 9.92 (s, 1H), 8.0 (s, 1H), 7.50 (d, J=1.89, 2H), 7.28 (t, J=1.91, 1H), 3.81 (s, 3H), 2.53 (s, 3H).

### (iv) Synthesis of 3-acetyl-6-(3,5-dichlorophenyl)-4H-thieno[3,2-b]pyrrole-5-carboxylic acid

[chemical structure]

The compound was prepared from methyl 3-acetyl-6-(3,5-dichlorophenyl)-4H-thieno[3,2-b]pyrrole-5-carboxylate (11 mg) following the General Method B for Ester Hydrolysis. Yield 10 mg (100%). ¹H NMR (CD₃OD): 8.49 (s, 1H), 7.64 (d, J=1.91, 2H), 7.41 (t, J=1.89), 1H), 2.61 (s, 3H).

### Example 22—Preparation of 3-acetyl-6-(3-chloro-4-((methylsulfonyl)methyl)phenyl)-4H-thieno[3,2-b]pyrrole-5-carboxylic acid

### (i) Synthesis of methyl 3-acetyl-6-(3-chloro-4-((methylsulfonyl)methyl)phenyl)-3a,6a-dihydro-4H-thieno[3,2-b]pyrrole-5-carboxylate

[chemical structure]

Methyl 3-acetyl-6-iodo-4H-thieno[3,2-b]pyrrole-5-carboxylate (205 mg, 0.587 mmol), 2-(3-chloro-4-((methylsulfonyl)methyl)phenyl)-4,4,5,5-tetramethyl-1,3,2-dioxaborolane (233 mg, 0.705 mmol), Pd(dppf)Cl₂— DCM (20.6 mg, 0.0252 mmol) and sodium carbonate (249 mg, 2.35 mmol) were mixed in deoxygenated dioxane (5 ml+10% water). The mixture was heated at 100° C. for 3 hours. The crude product was evaporated onto silica and purified by flash chromatography (20-100% ethyl acetate in pentane to give the target compound (106 mg, 42%) TLC, ethyl acetate-isohexane 1:1. Rf=0.3. ¹H NMR (CDCl₃): 10.0 (s, 1H), 8.08 (s, 1H), 7.86 (d, J=1.47, 1H), 7.66 (m, 2H), 4.53 (s, 2H), 3.88 (s, 3H), 2.89 (s, 3H), 2.61 (s, 3H).

### (ii) Synthesis of 3-acetyl-6-(3-chloro-4-((methylsulfonyl)methyl)phenyl)-4H-thieno[3,2-b]pyrrole-5-carboxylic acid

[chemical structure]

Prepared from methyl 3-acetyl-6-(3-chloro-4-((methylsulfonyl)methyl)phenyl)-3a,6a-dihydro-4H-thieno[3,2-b]pyrrole-5-carboxylate (100 mg) following the General Method B for Ester Hydrolysis. Yield: 62 mg, 60%. ¹H NMR (CD₃OD): 8.49 (s, 1H), 7.86 (d, J=1.76 Hz, 1H), 7.69 (dd, J=8.0 and 1.79 Hz, 1H), 7.63 (d, J=8.01, 1H), 4.68 (s, 2H), 2.99 (s, 3H), 2.61 (2.3H).

### Example 23—Preparation of 6-acetyl-3-(3,5-dichlorophenyl)-2-methyl-4H-thieno[3,2-b]pyrrole-5-carboxylic acid

### (i) Synthesis of methyl 6-acetyl-3-iodo-2-methyl-4H-thieno[3,2-b]pyrrole-5-carboxylate

[chemical structure]

Methyl 6-acetyl-2-methyl-4H-thieno[3,2-b]pyrrole-5-carboxylate (34 mg, 0.143 mmol) and N-iodosuccinimide (35.5 mg, 0.158 mmol) were dissolved in DMF and stirred at RT overnight. Extraction with ethyl acetate gave the target compound (17 mg, 33%). ¹H NMR (CDCl₃): 9.14 (br s, 1H), 3.06 (s, 3H), 2.78 (s, 3H), 2.51 (s, 3H).

### (ii) Synthesis of methyl 6-acetyl-3-(3,5-dichlorophenyl)-2-methyl-4H-thieno[3,2-b]pyrrole-5-carboxylate

[chemical structure]

Prepared from methyl 6-acetyl-3-iodo-2-methyl-4H-thieno[3,2-b]pyrrole-5-carboxylate (17 mg, 0.0629 mmol) and 3,5-dichlorophenyl boronic acid following the General Method B for Suzuki Coupling. Yield 11 mg, 61%. ¹H NMR (CDCl₃): 9.23 (br s, 1H), 7.4 (t, J=1.89 Hz, 1H), 7.29 (d, J=1.91, 2H), 3.97 (s, 3H), 2.79 (s, 3H), 2.53 (s, 3H).

### (iii) Synthesis of 6-acetyl-3-(3,5-dichlorophenyl)-2-methyl-4H-thieno[3,2-b]pyrrole-5-carboxylic acid

[chemical structure]

Prepared from methyl 6-acetyl-3-(3,5-dichlorophenyl)-2-methyl-4H-thieno[3,2-b]pyrrole-5-carboxylate (11 mg), following the General Method B for Ester Hydrolysis. Purified by flash chromatography (ethyl acetate-isohexane, 1:4). Yield 3 mg, 28%. ¹H NMR (CD₃OD): 9.84 (br s, 1H), 7.46 (t, J=1.89, 1H), 7.29 (d, J=1.90, 2H), 2.76 (s, 3H), 2.58 (s, 3H).

### Example 24—Preparation of (E)-6-(3-chloro-4-((methylsulfonyl)methyl)phenyl)-3-(1-((2-(dimethylamino)ethoxy)imino)ethyl)-4H-thieno[3,2-b]pyrrole-5-carboxylic acid

### (i) Synthesis of (E)-6-(3-chloro-4-((methylsulfonyl)methyl)phenyl)-3-(1-((2-(dimethylamino)ethoxy)imino)ethyl)-4H-thieno[3,2-b]pyrrole-5-carboxylic acid

[chemical structure]

3-acetyl-6-(3-chloro-4-((methylsulfonyl)methyl)phenyl)-4H-thieno[3,2-b]pyrrole-5-carboxylic acid (52 mg, 0.126 mmol), 2-(aminooxy)-N,N-dimethylethan-1-amine dihydrochloride (56 mg, 0.316 mmol) and potassium carbonate (87 mg, 0.63 mmol) were dissolved in ethanol (2 ml) and heated at 90° C. in a sealed tube over-night. Water was added and the mixture was extracted with ethyl acetate. Purification by preparative HPLC. 30-60% Acetonitrile in water, 15 min gradient, flow: 10 ml/min. buffer: TFA 0.1%. Nucleodur C18 column, 5 μm gave the desired compound (20 mg, 31%). ¹H NMR (CD₃OD): 7.87 (d, J=1.78 Hz, 1H), 7.79 (s, 1H), 7.68 dd, J=8.02 and 1.81 Hz, 1H), 7.61 (d, J=8.06, 1H), 4.68 (br s, 2H), 4.57 (m, 2H), 3.58 (m, 2H), 3.02 (s, 3H), 2.99 (s, 6H), 2.30 (s, 3H).

### Example 25—Preparation of 6-(3,5-dichlorophenyl)-3-(2H-tetrazol-5-yl)-4H-thieno[3,2-b]pyrrole-5-carboxylic acid

### (i) Synthesis of ethyl 3-cyano-6-(3,5-dichlorophenyl)-4H-thieno[3,2-b]pyrrole-5-carboxylate

[chemical structure]

Ethyl 3-bromo-6-(3,5-dichlorophenyl)-4H-thieno-[3,2-b]pyrrole-5-carboxylate (111 mg) and copper cyanide (50 mg, 2 eq) was dissolved in DMF (3 ml) and heated at 155° C. overnight. Reaction mixture was then cooled to room temperature and diluted with water, acidified with 1N HCl (1 mL) and extracted with EtOAc. The combined extracts were washed with water and brine, dried over anhydrous magnesium sulfate, and then the solvent was removed in vacuo. Material obtained was dissolved in MeOH/acetonitrile and evaporated several times with silica. Purification on flash column (manual, ethylacetate/isohexane 1:9) gave product as a white solid (90 mg). LC-MS (ESI): M⁻ 363. ¹H NMR (400 MHz, Chloroform-d) δ 9.61 (s, 1H), 7.98 (s, 1H), 7.54 (d, J=1.9 Hz, 2H), 7.38 (t, J=1.9 Hz, 1H), 4.37 (q, J=7.2 Hz, 2H), 1.34 (t, J=7.1 Hz, 3H).

### (ii) Synthesis of ethyl 6-(3,5-dichlorophenyl)-3-(2H-tetrazol-5-yl)-4H-thieno[3,2-b]pyrrole-5-carboxylate

[chemical structure]

Ethyl 3-cyano-6-(3,5-dichlorophenyl)-4H-thieno[3,2-b]pyrrole-5-carboxylate (100 mg), azidotrimethylsilane (0.02 mL, 1 eq), tetrabutylammonium fluoride trihydrate 1M in THF (0.27 ml, 1 eq) and anhydrous THF (2 mL) were mixed in a microwave 5 ml glass vial. The vessel was sealed and heated under microwave irradiation at 110° C. for 20 min. The reaction mixture was diluted with ethyl acetate (40 mL) and then washed with 1M HCl (3×10 mL). The organic phase was washed with brine (10 mL), dried, filtered and concentrated, affording the expected crude tetrazole derivative as light beige solid (75 mg). Purified by recrystallisation from methanol to give 45 mg of pure material. ¹H NMR (400 MHz, Acetonitrile-d₃) δ 10.38 (s, 1H), 8.23 (d, J=2.0 Hz, 1H), 7.73 (d, J=1.9 Hz, 2H), 7.51 (t, J=1.9 Hz, 1H), 4.36 (q, J=7.1 Hz, 2H), 1.35 (t, J=7.1 Hz, 4H).

### (iii) Synthesis of 6-(3,5-dichlorophenyl)-3-(2H-tetrazol-5-yl)-4H-thieno[3,2-b]pyrrole-5-carboxylic acid

[chemical structure]

The reaction was performed according to General method for hydrolysis for ethyl 6-(3,5-dichlorophenyl)-3-(2H-tetrazol-5-yl)-4H-thieno[3,2-b]pyrrole-5-carboxylate as starting material (42 mg) and 2 h heating at 50° C. to give 12 mg of desired product. ¹H NMR (400 MHz, DMSO-d₆) δ 13.23 (s, 1H), 11.90 (s, 1H), 8.40 (s, 1H), 7.71 (s, 2H), 7.63 (s, 1H).

### Example 26—Preparation of 3-(1-(tert-butoxycarbonyl)-1,2,3,6-tetrahydropyridin-4-yl)-6-(3,5-dichlorophenyl)-4H-thieno[3,2-b]pyrrole-5-carboxylic acid

### (i) Synthesis of ethyl 3-(1-(tert-butoxycarbonyl)-1,2,3,6-tetrahydropyridin-4-yl)-6-(3,5-dichlorophenyl)-4H-thieno[3,2-b]pyrrole-5-carboxylate

[chemical structure]

The reaction was performed according to General method for Suzuki coupling for ethyl 3-bromo-6-(3,5-dichlorophenyl)-4H-thieno[3,2-b]pyrrole-5-carboxylate (180 mg) and N-Boc-1,2,3,6-tetrahydropyridine-4-boronic acid pinacol ester as starting materials with reaction time 3 h at 100° C. of microwave heating to give 196 mg of desired product (yield 88%). LC-MS (ESI): M⁻ 519. ¹H NMR (400 MHz, Chloroform-d) δ 9.23 (s, 1H), 7.57 (d, J=1.9 Hz, 2H), 7.34 (t, J=1.9 Hz, 1H), 7.26 (s, 1H), 7.18 (s, 1H), 6.08 (s, 1H), 4.34 (q, J=7.1 Hz, 2H), 4.17 (d, J=3.2 Hz, 2H), 3.70 (dt, J=14.2, 5.9 Hz, 5H), 2.57 (s, 2H), 2.44 (t, J=6.2 Hz, 3H), 1.51 (s, 7H), 1.49 (s, 5H), 1.32 (t, J=7.1 Hz, 3H).

### (ii) Synthesis of 3-(1-(tert-butoxycarbonyl)-1,2,3,6-tetrahydropyridin-4-yl)-6-(3,5-dichlorophenyl)-4H-thieno[3,2-b]pyrrole-5-carboxylic acid

[chemical structure]

The reaction was performed according to General method for hydrolysis for ethyl 3-(1-(tert-butoxycarbonyl)-1,2,3,6-tetrahydropyridin-4-yl)-6-(3,5-dichlorophenyl)-4H-thieno[3,2-b]pyrrole-5-carboxylate as starting material (20 mg) to give 12 mg of desired product. LC-MS (ESI): M⁻ 491. ¹H NMR (400 MHz, Methanol-d₄) δ 7.51 (d, J=1.9 Hz, 2H), 7.29 (t, J=1.9 Hz, 1H), 7.25 (s, 1H), 6.22 (s, 1H), 4.05 (s, 2H), 3.58 (s, 2H), 2.49 (d, J=7.1 Hz, 2H), 1.41 (s, 9H).

### Example 27—Preparation of 3-(1-(tert-butoxycarbonyl)piperidin-4-yl)-6-(3,5-dichlorophenyl)-4H-thieno[3,2-b]pyrrole-5-carboxylic acid

### (i) Synthesis of ethyl 3-(1-(tert-butoxycarbonyl)piperidin-4-yl)-6-(3,5-dichlorophenyl)-4H-thieno[3,2-b]pyrrole-5-carboxylate

[chemical structure]

Ethyl 3-(1-(tert-butoxycarbonyl)-1,2,3,6-tetrahydropyridin-4-yl)-6-(3,5-dichlorophenyl)-4H-thieno[3,2-b]pyrrole-5-carboxylate (66 mg) was dissolved in methanol (4 ml), palladium on carbon (about 10-15 mg) was added and the reaction flask was placed in the hydrogenation autoclave, flushed with hydrogen and stirred under about 3 bar of hydrogen for 40 min. (Monitored by LC-MS: very low conversion). Hydrogenation was repeated at higher pressure (appr 5 bar) for 16 h and then for 24 h at maximum hydrogen pressure (about 7 bar). Conversion about 50% according to LC-MS. Reaction mixture was filtered through Celite and concentrated to give about 40 mg of material. The mixture was dissolved in methanol (about 4 ml) and palladium hydroxide on carbon (10 mg) was added and hydrogenation was repeated for total about 24 h (in 3 repetition with addition of extra catalyst). The final mixture contained less than 10% of starting material by LC-MS. Reaction mixture was filtered via Celite, concentrated and redissolved in acetonitrile with addition of water. Purified by prep chromatography to give 20 mg of desired product. ¹H NMR (400 MHz, Methanol-d₄) δ 11.78 (s, 1H), 7.56 (d, J=1.9 Hz, 2H), 7.36 (t, J=1.9 Hz, 1H), 7.08 (d, J=0.9 Hz, 1H), 4.29 (q, J=7.1 Hz, 2H), 4.21 (d, J=13.3 Hz, 2H), 3.05 (s, 1H), 2.93 (s, 2H), 2.08-1.96 (m, 2H), 1.64-1.52 (m, 3H), 1.47 (s, 7H), 1.30 (t, J=7.1 Hz, 3H). ¹³C NMR (101 MHz, Methanol-d₄) δ 161.34, 155.14, 139.60, 137.76, 134.08, 131.08, 127.58, 126.26, 125.11, 122.27, 122.06, 120.55, 79.66, 60.28, 35.29, 27.32, 13.15.

### (ii) Synthesis of 3-(1-(tert-butoxycarbonyl)piperidin-4-yl)-6-(3,5-dichlorophenyl)-4H-thieno[3,2-b]pyrrole-5-carboxylic acid

[chemical structure]

The reaction was performed according to General method for hydrolysis for ethyl 3-(1-(tert-butoxycarbonyl)piperidin-4-yl)-6-(3,5-dichlorophenyl)-4H-thieno[3,2-b]pyrrole-5-carboxylate as starting material (16 mg) to give 5 mg of desired product. LC-MS (ESI): M⁻ 493. ¹H NMR (400 MHz, Methanol-d₄) δ 7.53 (d, J=1.9 Hz, 2H), 7.26 (t, J=1.9 Hz, 1H), 7.00 (d, J=1.0 Hz, 1H), 4.12 (d, J=13.2 Hz, 3H), 3.88 (s, 1H), 3.04-2.91 (m, 2H), 2.85 (s, 3H), 2.24 (d, J=14.3 Hz, 1H), 1.95 (d, J=12.9 Hz, 3H), 1.59-1.42 (m, 4H), 1.38 (s, 9H), 1.20 (d, J=10.3 Hz, 2H), 0.86-0.74 (m, 1H).

### Example 28—Preparation of 6-(3,5-dichlorophenyl)-3-(1-(methylsulfonyl)piperidin-4-yl)-4H-thieno[3,2-b]pyrrole-5-carboxylic acid

### (i) Synthesis of ethyl 6-(3,5-dichlorophenyl)-3-(piperidin-4-yl)-4H-thieno[3,2-b]pyrrole-5-carboxylate

[chemical structure]

Ethyl 3-(1-(tert-butoxycarbonyl)piperidin-4-yl)-6-(3,5-dichlorophenyl)-4H-thieno[3,2-b]pyrrole-5-carboxylate (50 mg) was dissolved in 4M HCl in dioxane (4 ml) and stirred for about 4 h at r.t. Reaction mixture was concentrated by rotary evaporation, diluted with water, basified with 1N LiOH to pH9 and extracted into DCM, washed with brine, dried over magnesium sulfate. Concentration gave desired product as 35 mg of coloureless oil. LC-MS (ESI): M⁺ 377, 423, 464. ¹H NMR (400 MHz, Chloroform-d) δ 9.80 (s, 1H), 7.57 (t, J=1.6 Hz, 2H), 7.32 (q, J=2.0 Hz, 1H), 7.00 (s, 1H), 4.32 (qd, J=7.1, 0.9 Hz, 2H), 3.81-3.73 (m, 3H), 3.68-3.60 (m, 3H), 3.22 (t, J=13.3 Hz, 2H), 2.94-2.71 (m, 3H), 2.27-2.03 (m, 4H), 1.93 (h, J=12.4, 11.9 Hz, 5H), 1.31 (t, J=7.1 Hz, 4H), 1.25 (s, 2H).

### (ii) Synthesis of ethyl 6-(3,5-dichlorophenyl)-3-(1-(methylsulfonyl)piperidin-4-yl)-4H-thieno[3,2-b]pyrrole-5-carboxylate

[chemical structure]

Ethyl 6-(3,5-dichlorophenyl)-3-(piperidin-4-yl)-4H-thieno[3,2-b]pyrrole-5-carboxylate (35 mg) was dissolved in dry DCM, triethylamine (0,012 ml) was added and methylsulfonylchloride (10 microliter) was added to the stirred reaction mixture at r.t. and stirred overnight. Reaction mixture was concentrated, coevaporated 3 times with DCM and purified by column chromatography (Biotage Isolera GraceRes 4 g column EA/isohexane 0-50) to give 25 mg of pure material as white powder. ¹H NMR (400 MHz, Chloroform-d) δ 9.31 (s, 1H), 7.55 (d, J=1.9 Hz, 2H), 7.33 (t, J=1.9 Hz, 1H), 7.03 (s, 1H), 4.33 (q, J=7.1 Hz, 2H), 4.12 (q, J=7.1 Hz, 1H), 4.03-3.91 (m, 2H), 2.85 (d, J=3.0 Hz, 6H), 2.14 (d, J=13.1 Hz, 2H), 2.04 (s, 2H), 2.00-1.84 (m, 2H), 1.77-1.66 (m, 1H), 1.31 (t, J=7.1 Hz, 3H), 1.26 (t, J=7.1 Hz, 2H). ¹³C NMR (101 MHz, Chloroform-d) δ 161.44, 138.21, 136.57, 134.41, 129.22, 127.95, 127.34, 123.85, 122.15, 121.24, 61.18, 60.42, 46.24, 35.66, 35.06, 31.42, 14.21, 14.09.

### (iii) Synthesis of 6-(3,5-dichlorophenyl)-3-(1-(methylsulfonyl)piperidin-4-yl)-4H-thieno[3,2-b]pyrrole-5-carboxylic acid

[chemical structure]

The reaction was performed according to General method for hydrolysis for ethyl 6-(3,5-dichlorophenyl)-3-(1-(methylsulfonyl)piperidin-4-yl)-4H-thieno[3,2-b]pyrrole-5-carboxylate as starting material (23 mg) to give 13 mg of desired product. LC-MS (ESI): M⁻ 471. ¹H NMR (400 MHz, DMSO-d₆) δ 12.87 (s, 1H), 12.41 (s, 1H), 7.63 (d, J=2.0 Hz, 2H), 7.55 (t, J=2.0 Hz, 1H), 7.25 (s, 1H), 3.70 (d, J=11.6 Hz, 2H), 3.57 (s, 1H), 3.32 (s, 1H), 3.04 (t, J=12.0 Hz, 1H), 2.92 (s, 3H), 2.81 (t, J=11.3 Hz, 2H), 2.09 (d, J=12.2 Hz, 2H), 1.75-1.56 (m, 2H). ¹³C NMR (101 MHz, DMSO-d₆) δ 162.52, 139.48, 138.23, 134.04, 131.62, 128.03, 126.77, 124.60, 123.20, 122.74, 119.73, 66.82, 34.60, 34.50, 31.42.

### Example 29—Preparation of 6-(3,5-dichlorophenyl)-3-(5-(hydroxymethyl)-1,2,4-oxadiazol-3-yl)-4H-thieno[3,2-b]pyrrole-5-carboxylic acid

### (i) Synthesis of ethyl 6-(3,5-dichlorophenyl)-3-(N′-hydroxycarbamimidoyl)-4H-thieno[3,2-b]pyrrole-5-carboxylate

[chemical structure]

Ethyl 3-cyano-6-(3,5-dichlorophenyl)-4H-thieno[3,2-b]pyrrole-5-carboxylate (69 mg) was dissolved in ethanol in a 5 ml microwave vial, potassium carbonate (17 mg) and hydroxylamine hydrochloride (22 mg) were added to the reaction mixture heated in Biotage Initiator for 5 h at 100° C. (reaction was monitored by HPLC). Reaction mixture was concentrated by rotary evaporation to give beige powder (NMR for crude material). LC-MS (ESI): M⁺ 352/398/439. ¹H NMR (400 MHz, DMSO-d₆) δ 10.13 (s, 1H), 9.89 (s, 1H), 8.06 (s, 1H), 7.68 (d, J=1.9 Hz, 2H), 7.61 (t, J=1.9 Hz, 1H), 7.48 (s, 2H), 7.36 (s, 2H), 7.23 (s, 1H), 6.13 (s, 2H), 4.26 (q, J=7.1 Hz, 2H), 2.89 (s, 1H), 2.73 (s, 1H), 1.24 (t, J=7.1 Hz, 4H).

### (i) Synthesis of ethyl 3-(N′-(2-chloroacetoxy)carbamimidoyl)-6-(3,5-dichlorophenyl)-4H-thieno[3,2-b]pyrrole-5-carboxylate

[chemical structure]

Ethyl 6-(3,5-dichlorophenyl)-3-(N′-hydroxycarbamimidoyl)-4H-thieno[3,2-b]pyrrole-5-carboxylate (70 mg) was dissolved in DCM. Triethylamine (40 μL, 1.7 eq) was added via syringe and chloroacetyl chloride (20 μL, 1.5 eq) was added to the reaction and stirred overnight at r.t. Reaction mixture was concentrated by rotary evaporation, washed with brine and dried over magnesium sulfate. Concentration gave brownish solid. Material was dissolved in 4-5 ml of DCM and filtered to remove precipitate. Purified by flash chromatography on silica (GraceRes 4 g, Biotage Isolera, EA/iso-hexane gradient 5-20) to give desired material as 32 mg of white powder. ¹H NMR (400 MHz, Chloroform-d) δ 10.02 (s, 1H), 7.61 (s, 1H), 7.58 (d, J=1.9 Hz, 2H), 7.34 (t, J=1.9 Hz, 1H), 5.21 (s, 2H), 4.34 (q, J=7.1 Hz, 2H), 4.30 (s, 2H), 1.35 (t, J=7.1 Hz, 3H). ¹³C NMR (101 MHz, Chloroform-d) δ 164.37, 160.57, 151.57, 136.26, 136.22, 134.54, 127.84, 127.83, 127.42, 125.18, 122.96, 120.88, 116.43, 99.99, 61.14, 40.05, 14.13.

### (ii) Synthesis of ethyl 3-(5-(chloromethyl)-1,2,4-oxadiazol-3-yl)-6-(3,5-dichlorophenyl)-4H-thieno[3,2-b]pyrrole-5-carboxylate

[chemical structure]

Ethyl 3-(N′-(2-chloroacetoxy)carbamimidoyl)-6-(3,5-dichlorophenyl)-4H-thieno[3,2-b]pyrrole-5-carboxylate (30 mg) was dissolved in dry DMF in a 5 ml microwave vial and heated for 3 h at 120° C. Reaction mixture was diluted with water (20-30 ml) and extracted into ethyl acetate, washed with brine and dried over magnesium sulfate. Purification by flash chromatography on silica (GraceRes 4 g column, Biotage Isolera, EA/isohexane 0-25% gradient) to give desired product as white powder (30 mg). ¹H NMR (400 MHz, Chloroform-d) δ 9.80 (s, 1H), 8.14 (s, 1H), 7.59 (d, J=1.9 Hz, 2H), 7.36 (t, J=1.9 Hz, 1H), 4.79 (s, 2H), 4.36 (q, J=7.1 Hz, 2H), 1.34 (t, J=7.1 Hz, 3H), 1.26 (d, J=1.8 Hz, 1H).

### (iii) Synthesis of 6-(3,5-dichlorophenyl)-3-(5-(hydroxymethyl)-1,2,4-oxadiazol-3-yl)-4H-thieno[3,2-b]pyrrole-5-carboxylic acid

[chemical structure]

The reaction was performed according to General method for hydrolysis for ethyl 3-(5-(chloromethyl)-1,2,4-oxadiazol-3-yl)-6-(3,5-dichlorophenyl)-4H-thieno[3,2-b]pyrrole-5-carboxylate as starting material (20 mg) to give 4 mg of desired product after two prep HPLC purifications. ¹H NMR (400 MHz, Acetone-d₆) δ 10.08 (s, 1H), 8.04 (s, 1H), 7.74 (dd, J=1.9, 0.4 Hz, 2H), 7.46 (t, J=1.9 Hz, 1H), 6.05 (s, 2H), 5.62 (s, 1H).

### Example 30—Preparation of 6-(3,5-dichlorophenyl)-3-(piperidin-4-yl)-4H-thieno[3,2-b]pyrrole

### (i) Synthesis of 3-(1-(tert-butoxycarbonyl)piperidin-4-yl)-6-(3,5-dichlorophenyl)-4H-thieno[3,2-b]pyrrole-5-carboxylic acid

[chemical structure]

The reaction was performed according to General method for hydrolysis ethyl 3-(1-(tert-butoxycarbonyl)piperidin-4-yl)-6-(3,5-dichlorophenyl)-4H-thieno[3,2-b]pyrrole-5-carboxylate as starting material (45 mg) to give 25 mg of desired material (recrystallized from DCM/MeOH). ¹H NMR (400 MHz, DMSO-d₆) δ 12.84 (s, 1H), 12.39 (s, 1H), 7.63 (d, J=1.9 Hz, 2H), 7.55 (t, J=2.0 Hz, 1H), 7.22 (s, 1H), 4.09 (d, J=12.8 Hz, 2H), 3.09 (dd, J=13.6, 10.1 Hz, 1H), 2.83 (s, 2H), 1.96 (d, J=12.0 Hz, 2H), 1.54-1.35 (m, 11H), 1.42 (s, 9H).

### (ii) Synthesis of 6-(3,5-dichlorophenyl)-3-(piperidin-4-yl)-4H-thieno[3,2-b]pyrrole

[chemical structure]

3-(1-(Tert-butoxycarbonyl)piperidin-4-yl)-6-(3,5-dichlorophenyl)-4H-thieno[3,2-b]pyrrole-5-carboxylic acid (25 mg) was mixed with 2 ml of dry DCM (only partially soluble) and TFA (0.5 ml) was added at stirring. All insoluble material dissolved and colour changed to red brown. No starting material according to LC-MS after 30 min. Reaction mixture was poured into sodium bicarbonate solution and extracted into ethyl acetate (4×30 ml). Organic extracts washed with brine and dried over magnesium sulfate. Organic extracts were combined and concentrated by rotary evaporation and the crude material obtained was purified by preparative HPLC (dissolved in water/acetonitrile, flow rate 15 ml/min, 10 min gradient 20-80 pure MeCN/water with no additives). Obtained pure fractions were freeze dried to give 7 mg of material as white powder.). LC-MS (ESI): M⁺ 351/392. ¹H NMR (400 MHz, DMSO-d₆) δ 11.85 (s, 1H), 7.92-7.87 (m, 1H), 7.56 (d, J=1.9 Hz, 2H), 7.34 (t, J=1.9 Hz, 1H), 6.97 (s, 1H), 3.22-3.08 (m, 2H), 2.97-2.85 (m, 1H), 2.81-2.68 (m, 2H), 1.98 (d, J=11.8 Hz, 2H), 1.67 (qd, J=12.4, 3.8 Hz, 2H).

### Example 31—Preparation of 6-(3-chloro-4-((methylsulfonyl)methyl)phenyl)-3-isopropyl-1-methyl-1,2-dihydropyrrolo[3,2-c]pyrazole-5-carboxylic acid

### (i) Synthesis of 1-Benzyl-3-isopropyl-1H-pyrazole-5-carbaldehyde (A) and 1-Benzyl-5-isopropyl-1H-pyrazole-3-carbaldehyde (B)

[chemical structure]

5-isopropyl-1H-pyrazole-3-carbaldehyde (200 mg) and cesium carbonate (1.18 g, 2.5 eq) were mixed with 7 ml of dry DMF and benzyl bromide (0.2 ml, 1.05 eq) was added to the stirred reaction mixture at r.t. Reaction mixture was stirred overnight at r.t. and then diluted with water (100 ml) and extracted into EtOAc (4×35 ml). Organic extract was washed with water and brine and dried over magnesium sulphate. Crude material obtained after concentration by rotary evaporator was purified using Biotag Isolera (25 g KPSil column, 0-10% EA in isohexane) to give two isomeric products: 1-benzyl-3-isopropyl-1H-pyrazole-5-carbaldehyde (A)—126 mg (38%)¹H NMR (400 MHz, Chloroform-d) δ 9.77 (s, 1H), 7.42-7.18 (m, 7H), 6.78-6.69 (m, 1H), 5.66 (s, 2H), 3.04 (p, J=7.0 Hz, 1H), 1.29 (d, J=6.9 Hz, 6H) and 1-benzyl-5-isopropyl-1H-pyrazole-3-carbaldehyde (B)—193 mg (62%)¹H NMR (400 MHz, Chloroform-d) δ 9.95 (s, 1H), 7.42-7.28 (m, 3H), 7.16-7.06 (m, 2H), 6.65 (s, 1H), 5.41 (s, 2H), 2.89 (p, J=6.8 Hz, 1H), 1.17 (d, J=6.8 Hz, 6H).

### (ii) Synthesis of 1-benzyl-4-iodo-3-isopropyl-1H-pyrazole-5-carbaldehyde: [General Procedure for Iodination of Pyrazole Carbaldehyde]

[chemical structure]

1-benzyl-3-isopropyl-1H-pyrazole-5-carbaldehyde (126 mg) was dissolved in TFA (3 ml) and equimolar amount of NIS (128 mg) was added in one portion to the stirred reaction mixture. Reaction mixture was stirred at r.t. for 3 h (TLC), concentrated by rotary evaporation, mixed with solution of sodium bicarbonate solution and extracted into EtOAc (4×30 ml), washed with water and brine. Organic solution was dried over MgSO₄. Concentrated and purified by Biotage Isolera flash (10 g KP column 0-20% EA in isohexane) to give 173 mg of pure compound as light white crystals. ¹H NMR (400 MHz, Chloroform-d) δ 9.72 (s, 1H), 7.36-7.22 (m, 5H), 5.66 (s, 2H), 3.03 (p, J=6.9 Hz, 1H), 1.32 (d, J=6.9 Hz, 6H).

### (iii) Synthesis of ethyl 1-benzyl-3-isopropyl-1,4-dihydropyrrolo[3,2-c]pyrazole-5-carboxylate: [General Procedure for Formation of Pyrrolo[3,2-c]Pyrazole Cycle]

[chemical structure]

1-benzyl-4-iodo-3-isopropyl-1H-pyrazole-5-carbaldehyde (170 mg) was dissolved in abs DMF (3 ml) in a microwave 10 ml vial, cesium carbonate (2 eq) was added to the reaction mixture and flushed with nitrogen gas. CuI (20 mol %) was added to the reaction mixture, flushed with nitrogen, the vial was sealed and isocyanate (1 eq) was added via syringe. Reaction mixture was heated at 90° C. overnight. Reaction mixture was diluted with sat ammonium chloride solution and extracted into EA. Extracts were washed with water and brine and dried over MgSO₄. Organic extract concentrated by rotary evaporation and purified using Biotage Isolera flash chromatography on 12 g column (EA/isohexane 5-50%) to give 48 mg of desired product as slightly yellow powder. ¹H NMR (400 MHz, Chloroform-d) δ 8.44 (d, J=15.4 Hz, 1H), 7.40-7.18 (m, 6H), 6.29 (d, J=1.6 Hz, 1H), 5.31 (s, 2H), 4.33 (q, J=7.1 Hz, 2H), 3.19 (p, J=6.9 Hz, 1H), 1.40 (d, J=6.9 Hz, 6H), 1.35 (t, J=7.1 Hz, 3H).

### (iv) Synthesis of ethyl 1-benzyl-6-iodo-3-isopropyl-1,4-dihydropyrrolo[3,2-c]pyrazole-5-carboxylate: [General Procedure for Iodination of Pyrrolo[3,2-c]Pyrazole]

[chemical structure]

Ethyl 1-benzyl-3-isopropyl-1,4-dihydropyrrolo[3,2-c]pyrazole-5-carboxylate (48 mg) was dissolved in abs DMF (3 ml) and equimolar amount of NIS (35 mg) was added in one portion to the reaction mixture which was allowed to stir at r.t. for 2 h (reaction was monitored by TLC). Reaction mixture was poured into water (approx. 70 ml) and extracted into EtOAc (4×25 ml), washed with water and brine. The organic solution was dried over MgSO₄ and concentrated. Purified by Biotage Isolera flash (12 g Grace silica column 20-100% EA in isohexane, applied as DCM solution) to give 56 mg of product. ¹H NMR (400 MHz, Chloroform-d) δ 8.68 (s, 1H), 7.33-7.16 (m, 6H), 5.52 (s, 2H), 4.41 (q, J=7.1 Hz, 2H), 3.17 (p, J=7.0 Hz, 1H), 1.47-1.33 (m, 9H). ¹³C NMR (101 MHz, Chloroform-d) δ 161.40, 140.94, 139.34, 138.13, 128.96, 128.59, 127.55, 127.32, 126.58, 61.44, 52.32, 45.74, 27.66, 22.38, 14.52.

### (v) Synthesis of Ethyl 1-benzyl-6-(3-chloro-4-((methylsulfonyl)methyl)phenyl)-3-isopropyl-1,4-dihydropyrrolo[3,2-c]pyrazole-5-carboxylate: [General Procedure for Suzuki Coupling]

[chemical structure]

Ethyl-1-benzyl-6-iodo-3-isopropyl-1,4-dihydropyrrolo[3,2-c]pyrazole-5-carboxylate (55 mg) and 2-(3-chloro-4-((methylsulfonyl)methyl)phenyl)-4,4,5,5-tetramethyl-1,3,2-dioxaborolane (50 mg) were dissolved in 1,4-dioxane (about 3 ml) and bubbled with nitrogen in a MW vial, sodium carbonate (27 mg) was dissolved in distilled water (0.5 ml) and added to the reaction mixture via syringe. Catalyst (about 10 mg) was added to the reaction mixture, bubbled with nitrogen and heated for about 55 min at 110° C. (LC-MS). Reaction mixture was poured into sat. ammonium chloride (60 ml) and extracted into ethyl acetate (4×30 ml), washed with water and brine and dried over magnesium sulfate. Purified by flash chromatography on silica Biotage Isolera (12 g GraceRes column, 20-80 EA/isohexane). Desired material was obtained as 51 mg of an oil/foam. ¹H NMR (400 MHz, Chloroform-d) δ 8.53 (s, 1H), 7.50 (d, J=8.0 Hz, 1H), 7.34 (d, J=1.7 Hz, 1H), 7.21 (dd, J=7.9, 1.8 Hz, 1H), 7.14 (dd, J=5.0, 1.9 Hz, 3H), 6.72-6.65 (m, 2H), 5.15 (s, 2H), 4.51 (s, 2H), 4.20 (q, J=7.1 Hz, 2H), 3.24 (p, J=7.0 Hz, 1H), 2.85 (d, J=0.7 Hz, 3H), 1.45 (d, J=6.9 Hz, 6H), 1.14 (t, J=7.1 Hz, 3H). ¹³C NMR (101 MHz, Chloroform-d) δ 161.96, 139.38, 137.47, 135.80, 133.57, 132.21, 132.04, 129.95, 128.54, 127.49, 126.69, 126.33, 125.72, 125.40, 61.10, 57.88, 54.13, 39.97, 27.65, 22.48, 14.12.

### (vi) Synthesis of 4-(tert-butyl) 5-ethyl 1-benzyl-6-(3-chloro-4-((methylsulfonyl)methyl)phenyl)-3-isopropylpyrrolo[3,2-c]pyrazole-4,5(1H)-dicarboxylate: [General Procedure for N-Boc-Protection of Pyrrolo[3,2-c]Pyrazole]

[chemical structure]

Ethyl-1-benzyl-6-(3-chloro-4-((methylsulfonyl)methyl)phenyl)-3-isopropylpyrrolo[3,2c]pyrazole-5-carboxylate (50 mg) was dissolved in dry acetonitrile (3 ml) and Boc anhydride (30 mg) together with catalytic amount of DMAP (4 mg) was added to the reaction mixture and stirred overnight at r.t. Reaction mixture was concentrated by rotary evaporation and distributed between ethylacetate and water. Water phase extracted into EtOAc (3×25 ml). Combined organic phase washed with brine, dried over magnesium sulfate and concentrated. Purified by column chromatography on silica (Biotage Isolera, 10 g column, EA/isohexane 0-30%) to give 49 mg of desired product. ¹H NMR (400 MHz, Chloroform-d) δ 7.42 (d, J=7.9 Hz, 1H), 7.21 (d, J=1.7 Hz, 1H), 7.11-7.04 (m, 4H), 6.62-6.55 (m, 2H), 5.09 (s, 2H), 4.42 (s, 2H), 4.08 (q, J=7.1 Hz, 2H), 3.63 (hept, J=6.9 Hz, 1H), 2.78 (s, 3H), 1.53 (s, 9H), 1.32 (d, J=6.9 Hz, 6H), 1.04 (t, J=7.1 Hz, 3H).

### (vii) Synthesis of 4-(tert-butyl) 5-ethyl 6-(3-chloro-4-((methylsulfonyl)methyl)phenyl)-3-isopropylpyrrolo[3,2-c]pyrazole-4,5(1H)-dicarboxylate: [General Procedure for Benzyl Deprotection (Catalytic Hydrogenation)]

[chemical structure]

4-(Tert-butyl)-5-ethyl-1-benzyl-6-(3-chloro-4-((methylsulfonyl)methyl)phenyl)-3-isopropylpyrrolo[3,2-c]pyrazole-4,5(1H)-dicarboxylate (49 mg) was dissolved in methanol (10 ml), 1 ml of 1M HCl solution was added to the reaction mixture. Reaction mixture was flushed with nitrogen gas (3×), then palladium hydroxide on carbon (about 10 mg, 10%) was added and the reaction flask was flushed with hydrogen (3×) and stirred for 16 h under hydrogen atmosphere at r.t. and normal pressure. Reaction mixture was filtered through Celite (washed with methanol) and concentrated. Purified by column chromatography on 10 g silica column (EA/isohexane 30-100 gradient) to give desired material 22.5 mg (LC-MS: M⁺ 466, 524, 565) together with 27 mg of dichlorinated product 4-(tert-butyl) 5-ethyl 3-isopropyl-6-(4-((methylsulfonyl)methyl)phenyl)pyrrolo[3,2-c]pyrazole-4,5(1H)-dicarboxylate.

### (viii) Synthesis of 4-(Tert-butyl) 5-ethyl 6-(3-chloro-4-((methylsulfonyl)methyl)phenyl)-3-isopropyl-1-methyl pyrrolo[3,2-c]pyrazole-4,5(1H)-dicarboxylate. [General Procedure A for Methylation of Pyrrolo[3,2-c]Pyrazole]

[chemical structure]

4-(Tert-butyl)-5-ethyl-6-(3-chloro-4-((methylsulfonyl)methyl)phenyl)-3-isopropylpyrrolo[3,2-c]pyrazole-4,5(1H)-dicarboxylate (22 mg) and cesium carbonate (14 mg, 1 eq) was dissolved in dry DMF and Mel (2 microliters, 1 eq) was added to the reaction mixture and stirred overnight at r.t. (LC-MS). Reaction mixture was mixed with water and extracted into EtOAc (4×25 ml), washed with brine, dried over MgSO₄. Purified by column chromatography on 4 g flash column (Biotage, 0-50 EA/isohexane) to give about 20 mg of desired product. LC-MS: M⁺ 538, 579.

### General Method C for Ester Hydrolysis

Ester (20-60 mg) was dissolved in dioxane (0.5 ml) and 1N solution of LiOH in water (0.3 ml) was added to the reaction mixture, diluted with distilled water (about 0.3 ml) and stirred at 60-75° C. for 2-3 h. Reaction was monitored by LC-MS. Reaction mixture was poured into water (about 10 ml) and extracted into hexane. Water phase was acidified to pH 1 with 1N HCl and extracted into ethyl acetate (4×20 ml). Organic extract was washed with brine and dried over magnesium sulfate. Crude product was purified by preparative HPLC (flow rate 15 ml/min, gradient 20-90% MeCN in water with 0.1% of formic acid). Pure fractions collected were combined and concentrated by rotary evaporation. Prepared material was freeze dried from 1:1 mixture of acetonitrile and water and dried in high vacuum in presence of P₂O₅ if necessary.

### (ix) Synthesis of 6-(3-chloro-4-((methylsulfonyl)methyl)phenyl)-3-isopropyl-1-methyl-1,2-dihydropyrrolo[3,2-c]pyrazole-5-carboxylic acid

[chemical structure]

Compound was prepared according to General Method C for Ester Hydrolysis from 20 mg of 4-(tert-butyl)-5-ethyl-6-(3-chloro-4-((methylsulfonyl)methyl)phenyl)-3-isopropyl-1-methyl pyrrolo[3,2-c]pyrazole-4,5(1H)-dicarboxylate to give 6.3 mg of desired product. LC-MS: M⁺ 410.0. ¹H NMR (400 MHz, DMSO-d₆) δ 11.48 (s, 1H), 7.66 (s, 1H), 7.56 (d, J=8.0 Hz, 1H), 7.51 (d, J=7.9 Hz, 1H), 4.69 (s, 2H), 3.56 (s, 3H), 3.14-3.00 (m, 1H), 3.05 (s, 3H), 1.31 (d, J=6.9 Hz, 9H). ¹³C NMR (101 MHz, DMSO) δ 162.89, 138.08, 137.63, 135.20, 133.46, 132.34, 131.66, 129.62, 125.82, 125.12, 108.12, 56.57, 37.12, 26.81, 21.88.

### Example 32 and 33—Preparation of 3-Isopropyl-2-methyl-6-(4-((methylsulfonyl)methyl)phenyl)-2,4-dihydropyrrolo[3,2-c]pyrazole-5-carboxylic acid and 3-Isopropyl-1-methyl-6-(4-((methylsulfonyl)methyl)phenyl)-1,4-dihydropyrrolo[3,2-c]pyrazole-5-carboxylic acid

### (i) Synthesis of 4-(tert-butyl) 5-ethyl 3-isopropyl-6-(4-((methylsulfonyl)methyl)phenyl)pyrrolo[3,2-c]pyrazole-4,5(1H)-dicarboxylate

[chemical structure]

27 mg (LC-MS: M⁺ 431, 490, 531) of the compound was obtained as byproduct in catalytic hydrogenation of 4-(tert-butyl)-5-ethyl-1-benzyl-6-(3-chloro-4-((methylsulfonyl)methyl)phenyl)-3-isopropylpyrrolo[3,2-c]pyrazole-4,5(1H)-dicarboxylate.

### (ii) Synthesis of 4-(tert-butyl) 5-ethyl 6-(3-chloro-4-((methylsulfonyl)methyl)phenyl)-3-isopropyl-2-methylpyrrolo[3,2-c]pyrazole-4,5(2H)-dicarboxylate

[chemical structure]

Compound was prepared according to General Procedure for N-Boc-protection of pyrrolo[3,2-c]pyrazole from 53 mg of ethyl 6-(3-chloro-4-((methylsulfonyl)methyl)phenyl)-3-isopropyl-2-methyl-2,4-dihydropyrrolo[3,2-c]pyrazole-5-carboxylate to give 40 mg of desired product (used without additional purification). LC-MS: M⁺ 479, 538, 579.

### (iii) Synthesis of 4-(tert-butyl) 5-ethyl 3-isopropyl-2-methyl-6-(4-((methylsulfonyl)methyl)phenyl)pyrrolo[3,2-c]pyrazole-4,5(2H)-dicarboxylate

[chemical structure]

Compound was prepared according to general procedure A for methylation of pyrrolo[3,2-c]pyrazole from 27 mg of 4-(tert-butyl)-5-ethyl-3-isopropyl-6-(4-((methylsulfonyl)methyl)phenyl)pyrrolo[3,2-c]pyrazole-4,5(2H)-dicarboxylate to give 20 mg of desired product. LC-MS: M⁺ 479, 538, 579.

### (iv) Synthesis of 3-Isopropyl-2-methyl-6-(4-((methylsulfonyl)methyl)phenyl)-2,4-dihydropyrrolo[3,2-c]pyrazole-5-carboxylic acid (A) and 3-Isopropyl-1-methyl-6-(4-((methylsulfonyl)methyl)phenyl)-1,4-dihydropyrrolo[3,2-c]pyrazole-5-carboxylic acid (B)

[chemical structure]

Compounds were prepared according to General Method C for Ester Hydrolysis from 27 mg of mixture of corresponding ethyl esters at room temperature for 96 h to give 2 mg of each product after separation and purification by preparative HPLC. Compound A: LC-MS: M⁺ 376.0, 417, 751; ¹H NMR (400 MHz, DMSO-d₆) δ 10.60 (s, 1H), 7.88 (d, J=8.0 Hz, 2H), 7.37 (d, J=8.2 Hz, 2H), 4.47 (s, 2H), 3.93 (s, 3H), 2.93 (s, 3H), 1.36 (d, J=6.9 Hz, 6H). ¹³C NMR (101 MHz, DMSO) δ 163.96, 161.66, 149.24, 139.75, 138.65, 134.84, 134.69, 133.37, 132.16, 131.71, 129.44, 129.13, 128.16, 125.97, 124.66, 56.51, 48.83, 48.60, 34.82, 25.62, 20.59. Compound B: LC-MS: M⁺ 376.0, 417; ¹H NMR (400 MHz, DMSO-d6) δ 11.19 (s, 1H), 7.50 (d, J=8.1 Hz, 2H), 7.40 (d, J=8.1 Hz, 2H), 4.51 (s, 2H), 3.52 (s, 3H), 3.14-2.84 (m, 3H), 1.30 (d, J=7.0 Hz, 6H). ¹³C NMR (101 MHz, DMSO) δ 163.38, 137.96, 130.92, 130.05, 127.15, 99.59, 59.29, 37.04, 26.82, 21.92.

### Example 34—Preparation of 2-benzyl-6-(3-chloro-4-((methylsulfonyl)methyl)phenyl)-3-isopropyl-1-methyl-2,4-dihydro-1I4-pyrrolo[2,3-d]pyrazole-5-carboxylic acid

### (i) Synthesis of 1-benzyl-4-iodo-5-isopropyl-1H-pyrazole-3-carbaldehyde

[chemical structure]

Compound was prepared according to General Procedure for Iodination of pyrazole carbaldehyde from 193 mg of 1-benzyl-5-isopropyl-1H-pyrazole-3-carbaldehyde to give 257 mg of desired product. ¹H NMR (400 MHz, Chloroform-d) δ 9.88 (s, 1H), 7.32-7.19 (m, 3H), 7.02 (ddt, J=7.2, 1.4, 0.7 Hz, 2H), 5.40 (s, 2H), 3.11 (hept, J=7.2 Hz, 1H), 1.18 (d, J=7.2 Hz, 6H). ¹³C NMR (101 MHz, Chloroform-d) δ 186.07, 149.46, 147.83, 135.65, 128.99, 128.28, 126.65, 55.51, 26.81, 20.12.

### (ii) Synthesis of ethyl 2-benzyl-3-isopropyl-2,4-dihydropyrrolo[3,2-c]pyrazole-5-carboxylate

[chemical structure]

Compound was prepared according to general procedure for formation of pyrrolo[3,2-c]pyrazole cycle from 255 mg of 1-benzyl-4-iodo-5-isopropyl-1H-pyrazole-3-carbaldehyde to give 105 mg of desired product. ¹H NMR (400 MHz, Chloroform-d) δ 7.88 (s, 1H), 7.29-7.13 (m, 2H), 7.04-6.97 (m, 2H), 6.82 (d, J=1.5 Hz, 1H), 5.41 (s, 2H), 4.32 (q, J=7.1 Hz, 2H), 3.05 (hept, J=6.9 Hz, 1H), 1.33 (t, J=7.1 Hz, 3H), 1.22 (d, J=6.9 Hz, 6H). ¹³C NMR (101 MHz, Chloroform-d) δ 162.39, 147.64, 137.30, 133.31, 128.76, 127.68, 126.54, 125.88, 99.59, 61.02, 54.75, 25.65, 22.16, 14.43.

### (i) Synthesis of ethyl 2-benzyl-6-iodo-3-isopropyl-2,4-dihydropyrrolo[3,2-c]pyrazole-5-carboxylate

[chemical structure]

Compound was prepared according to General Procedure for iodination of pyrrolo[3,2-c]pyrazole from 105 mg of ethyl 2-benzyl-3-cyclopropyl-6-iodo-2,4-dihydropyrrolo[3,2-c]pyrazole-5-carboxylate to give 116 mg of desired product. LC-MS: M⁺ 438. ¹H NMR (400 MHz, Chloroform-d) δ 8.25 (s, 1H), 7.38-7.22 (m, 4H), 7.12-7.02 (m, 1H), 5.52 (s, 2H), 4.44 (q, J=7.1 Hz, 2H), 3.06 (p, J=6.9 Hz, 1H), 1.46 (t, J=7.1 Hz, 3H), 1.24 (d, J=6.9 Hz, 6H). ¹³C NMR (101 MHz, Chloroform-d) δ 161.53, 150.78, 137.14, 132.29, 129.96, 128.86, 127.86, 126.63, 124.23, 61.57, 55.30, 53.25, 26.06, 22.16, 14.50.

### (ii) Synthesis of ethyl 2-benzyl-6-(3-chloro-4-((methylsulfonyl)methyl)phenyl)-3-isopropyl-2,4-dihydropyrrolo[3,2-c]pyrazole-5-carboxylate

[chemical structure]

Compound was prepared according to General Procedure for Suzuki coupling from 115 mg of ethyl 2-benzyl-6-iodo-3-isopropyl-2,4-di hydropyrrolo[3,2-c]pyrazole-5-carboxylate to give 114 mg of desired product. ¹H NMR (400 MHz, Chloroform-d) δ 8.16 (s, 1H), 8.04 (d, J=1.7 Hz, 1H), 7.92 (dd, J=8.1, 1.8 Hz, 1H), 7.62 (d, J=8.1 Hz, 1H), 7.33-7.22 (m, 5H), 7.10-7.04 (m, 2H), 5.52 (s, 2H), 4.50 (s, 2H), 4.37 (q, J=7.1 Hz, 2H), 3.12 (p, J=6.9 Hz, 1H), 2.83 (d, J=0.8 Hz, 3H), 1.34 (t, J=7.1 Hz, 3H), 1.30 (d, J=6.9 Hz, 6H). ¹³C NMR (101 MHz, Chloroform-d) δ 162.10, 147.70, 137.23, 136.05, 133.72, 132.45, 131.39, 129.54, 129.32, 128.90, 128.00, 127.88, 126.58, 124.84, 124.40, 113.33, 61.50, 58.06, 55.14, 39.69, 25.86, 22.31, 14.30.

### (iii) Synthesis of 2-Benzyl-6-(3-chloro-4-((methylsulfonyl)methyl)phenyl)-3-isopropyl-2,4-dihydropyrrolo[3,2-c]pyrazole-5-carboxylic acid

[chemical structure]

Compound was prepared according to General Procedure for ester hydrolysis from 10 mg of ethyl 2-benzyl-6-(3-chloro-4-((methylsulfonyl)methyl)phenyl)-3-isopropyl-2,4-dihydro pyrrolo[3,2-c]pyrazole-5-carboxylate to give 9 mg of desired product. No purification was performed for the crude product. LC-MS: M⁺ 486.0; M⁻ 484.0.

### (iv) Synthesis of 2-Benzyl-6-(3-chloro-4-((methylsulfonyl)methyl)phenyl)-3-isopropyl-1-methyl-2,4-dihydro-1I4-pyrrolo[2,3-d]pyrazole-5-carboxylic acid [General Procedure B for Methylation of Pyrrolo[3,2-c]Pyrazole]

[chemical structure]

2-benzyl-6-(3-chloro-4-((methylsulfonyl)methyl)phenyl)-3-isopropyl-2,4-dihydropyrrolo[3,2-c]pyrazole-5-carboxylic acid (9 mg) was dissolved in dry DMF (about 2 ml) and Mel (7 μl, 2.5 equiv.) was added to the reaction mixture and reaction mixture was heated at stirring for 20 h at 90° C. (monitored by LC-MS). Reaction mixture was concentrated by rotary evaporation, diluted with water purified by prep HPLC (flow rate 15 ml/min, gradient 20-90% MeCN in water with 0.1% of formic acid). Pure fractions collected were combined and concentrated by rotary evaporation. Prepared material was freeze dried from 1:1 mixture of acetonitrile and water and dried in high vacuum in presence of P₂O₅. Desired product was obtained as 2.7 mg of white light powder. ¹H NMR (400 MHz, DMSO-d6) δ 8.41 (s, OH), 7.74 (s, 1H), 7.51 (s, 2H), 7.41-7.28 (m, 3H), 7.18-7.07 (m, 2H), 5.84 (s, 2H), 4.66 (s, 2H), 3.70 (s, 3H), 3.17 (s, 4H), 3.03 (s, 3H), 1.34 (d, J=6.9 Hz, 6H). ¹³C NMR (101 MHz, DMSO) δ 163.96, 161.66, 149.24, 139.75, 138.65, 134.84, 134.69, 133.37, 132.16, 131.71, 129.44, 129.13, 128.16, 125.97, 124.66, 56.51, 48.83, 48.60, 34.82, 25.62, 20.59.

### Example 35—Preparation of 6-(3-chloro-4-((methylsulfonyl)methyl)phenyl)-3-isopropyl-2-methyl-2,4-dihydropyrrolo[3,2-c]pyrazole-5-carboxylic acid

### (i) Synthesis of 5-Isopropyl-1-methyl-1H-pyrazole-3-carbaldehyde

[chemical structure]

Sodium hydride (105 mg, 60% in mineral oil) was added to dry THF (20 ml) and stirred at r.t. for about 10 min. 5-Isopropyl-1H-pyrazole-3-carbaldehyde (300 mg) was added to the stirred reaction mixture at r.t. Reaction mixture was stirred under nitrogen for about 15 min and then Mel (0.14 ml) was added via syringe and stirred overnight at r.t. Reaction mixture was diluted with water (150 ml) and extracted into EtOAc (4×30 ml). Organic extract was washed with water and brine and dried over magnesium sulphate. Crude material obtained after concentration by rotary evaporation purified using Biotag Isolera (10 g GraceRes column, 0-10% EA in isohexane) to give desired compound as coloureless oil (87 mg). Position of benzyl was proved by 2D NOESY experiment. ¹H NMR (400 MHz, Chloroform-d) δ 9.88 (s, 1H), 6.58 (d, J=0.6 Hz, 1H), 3.91 (s, 3H), 2.95 (pd, J=6.8, 0.7 Hz, 1H), 1.28 (d, J=6.9 Hz, 6H).

### (ii) Synthesis of 4-iodo-5-isopropyl-1-methyl-1H-pyrazole-3-carbaldehyde

[chemical structure]

Compound was prepared according to general procedure for iodination of pyrazole carbaldehyde from 139 mg of 5-isopropyl-1-methyl-1H-pyrazole-3-carbaldehyde to give 187 mg of desired product. ¹H NMR (400 MHz, Chloroform-d) δ 9.87 (s, 1H), 4.00 (s, 3H), 3.32 (p, J=7.3 Hz, 1H), 1.40 (d, J=7.2 Hz, 6H).

### (iii) Synthesis of Ethyl 3-isopropyl-2-methyl-2,4-dihydropyrrolo[3,2-c]pyrazole-5-carboxylate

[chemical structure]

Compound was prepared according to general procedure for formation of pyrrolo[3,2-c]pyrazole cycle from 187 mg of 5-isopropyl-4-iodo-1-methyl-1H-pyrazole-3-carbaldehyde to give 67 mg of desired product. LC-MS: M⁺ 236. ¹H NMR (400 MHz, Chloroform-d) δ 7.97 (s, 1H), 6.84 (d, J=1.5 Hz, 1H), 4.37 (q, J=7.1 Hz, 2H), 3.99 (s, 3H), 3.19 (p, J=6.9 Hz, 1H), 1.46-1.32 (m, 9H). ¹³C NMR (101 MHz, Chloroform-d) δ 162.45, 147.21, 132.89, 128.30, 125.70, 99.27, 60.96, 37.90, 25.69, 21.90, 14.41.

### (iv) Synthesis of ethyl 6-iodo-3-isopropyl-2-methyl-2,4-dihydropyrrolo[3,2-c]pyrazole-5-carboxylate

[chemical structure]

Compound was prepared according to general procedure for iodination of pyrrolo[3,2-c]pyrazole from 68 mg of ethyl 3-isopropyl-2-methyl-2,4-di hydropyrrolo[3,2-c]pyrazole-5-carboxylate to give 64 mg of desired product. LC-MS: M⁺ 462. ¹H NMR (400 MHz, Chloroform-d) δ 8.25 (s, 1H), 4.42 (q, J=7.1 Hz, 2H), 4.02 (s, 3H), 3.18 (p, J=6.9 Hz, 1H), 1.45 (t, J=7.1 Hz, 3H), 1.40 (d, J=6.9 Hz, 6H). ¹³C NMR (101 MHz, Chloroform-d) δ 161.56, 150.42, 131.94, 129.72, 123.90, 61.54, 52.83, 38.24, 26.14, 21.93, 14.47.

### (v) Synthesis of ethyl 6-(3-chloro-4-((methylsulfonyl)methyl)phenyl)-3-isopropyl-2-methyl-2,4-dihydropyrrolo[3,2-c]pyrazole-5-carboxylate

[chemical structure]

Compound was prepared according to general procedure for Suzuki coupling from 63 mg of ethyl 6-iodo-3-isopropyl-2-methyl-2,4-di hydropyrrolo[3,2-c]pyrazole-5-carboxylate to give 53.3 mg of desired product. ¹H NMR (400 MHz, Chloroform-d) δ 8.14 (s, 1H), 8.00 (d, J=1.8 Hz, 1H), 7.87 (dd, J=8.1, 1.8 Hz, 1H), 7.61 (d, J=8.1 Hz, 1H), 4.50 (s, 2H), 4.35 (q, J=7.1 Hz, 2H), 4.03 (s, 3H), 3.23 (p, J=6.9 Hz, 1H), 2.83 (d, J=0.9 Hz, 3H), 1.45 (d, J=6.9 Hz, 6H), 1.33 (t, J=7.1 Hz, 3H). ¹³C NMR (101 MHz, Chloroform-d) δ 162.14, 147.36, 136.11, 133.72, 132.42, 131.30, 129.44, 129.02, 127.63, 124.80, 124.13, 113.06, 61.45, 58.05, 39.64, 38.20, 25.95, 22.07, 14.28.

### (vi) Synthesis of 6-(3-chloro-4-((methylsulfonyl)methyl)phenyl)-3-isopropyl-2-methyl-2,4-dihydropyrrolo[3,2-c]pyrazole-5-carboxylic acid

[chemical structure]

Compound was prepared according to general procedure for ester hydrolysis from 40 mg of ethyl 6-(3-chloro-4-((methylsulfonyl)methyl)phenyl)-3-isopropyl-2-methyl-2,4-dihydro pyrrolo[3,2-c]pyrazole-5-carboxylate at 55° C. for 2 h to give 18 mg of desired product after prep HPLC. LC-MS: M⁺ 330, 410, 451. ¹H NMR (400 MHz, DMSO-d6) δ 13.08 (s, 1H), 10.86 (s, 1H), 8.08 (d, J=1.8 Hz, 1H), 7.93 (dd, J=8.1, 1.8 Hz, 1H), 7.51 (d, J=8.2 Hz, 1H), 4.65 (s, 2H), 3.96 (s, 3H), 3.03 (s, 3H), 1.37 (d, J=6.9 Hz, 6H). 13C NMR (101 MHz, DMSO) δ 163.68, 146.82, 136.60, 133.99, 132.83, 130.21, 129.55, 128.87, 128.53, 124.43, 124.17, 110.63, 57.11, 38.34, 25.30, 21.88. Same product was obtained by hydrolysis of 35 mg of 4-(tert-butyl) 5-ethyl 6-(3-chloro-4-((methylsulfonyl)methyl)phenyl)-3-isopropyl-2-methylpyrrolo[3,2-c]pyrazole-4,5(2H)-dicarboxylate at 60° C. for 15 h yielding 22 mg after prep HPLC. LC-MS: M⁺ 451. M⁻ 408.

### Example 36—Preparation of 6-(3-Chloro-4-((methylsulfonyl)methyl)phenyl)-3-isopropyl-1,2-dimethyl-2,4-dihydro-1I4-pyrrolo[2,3-d]pyrazole-5-carboxylic acid

### (i) Synthesis of 6-(3-Chloro-4-((methylsulfonyl)methyl)phenyl)-3-isopropyl-1,2-dimethyl-2,4-dihydro-1I4-pyrrolo[2,3-d]pyrazole-5-carboxylic acid

[chemical structure]

Compound was prepared according to General Procedure B for methylation of pyrrolo[3,2-c]pyrazole from 20 mg of 6-(3-chloro-4-((methylsulfonyl)methyl)phenyl)-3-isopropyl-2-methyl-2,4-dihydropyrrolo[3,2-c]pyrazole-5-carboxylic acid to give 10 mg of desired product. ¹H NMR (400 MHz, DMSO-d6) δ 8.25 (s, 1H), 7.67 (d, J=1.7 Hz, 1H), 7.54 (d, J=8.0 Hz, 1H), 7.47 (dd, J=7.9, 1.8 Hz, 1H), 4.67 (s, 2H), 4.01 (s, 4H), 3.73 (s, 7H), 3.05 (s, 3H), 2.54 (s, 4H), 1.43 (d, J=6.9 Hz, 6H). ¹³C NMR (101 MHz, DMSO) δ 163.96, 162.30, 146.72, 138.57, 137.64, 134.65, 133.49, 132.32, 131.73, 129.43, 124.87, 119.47, 101.50, 56.57, 40.50, 40.43, 34.48, 33.52, 25.49, 20.38.

### Example 37—Preparation of 3-cyclopropyl-6-(4-fluorophenyl)-1,4-dihydropyrrolo[3,2-c]pyrazole-5-carboxylic acid

### (i) Synthesis of 1-benzyl-3-cyclopropyl-1H-pyrazole-5-carbaldehyde (A) and 1-Benzyl-5-cyclopropyl-1H-pyrazole-3-carbaldehyde (B)

[chemical structure]

3-Cyclopropyl-1H-pyrazole-5-carbaldehyde (440 mg) and cesium carbonate (2.6 g, 2.5 eq) were mixed with 7 ml of dry DMF and benzyl bromide (0.4 ml, 1.05 eq) was added to the stirred reaction mixture at r.t. Reaction mixture was stirred under nitrogen overnight, then diluted with water (150 ml) and extracted into EtOAc (4×35 ml). Organic extract washed with water and brine and dried over magnesium sulphate. Crude material obtained after concentration by rotary evaporator was purified using Biotage Isolera (10 g ULTRASil column, 0-10% EA in isohexane) to give isomeric products: 1-benzyl-3-cyclopropyl-1H-pyrazole-5-carbaldehyde (A)—221 mg (28%) of coloureless oil and 1-benzyl-5-cyclopropyl-1H-pyrazole-3-carbaldehyde (B)—402 mg (50%) of brownish oil. Position of benzyl was proved by 1D NOESY experiment on CH₂ signal of benzyl-group and 2D NOESY experiment. A: ¹H NMR (400 MHz, Chloroform-d) δ 9.72 (s, 1H), 7.41-7.12 (m, 5H), 6.55 (s, 1H), 5.62 (s, 2H), 1.96 (ddd, J=8.4, 5.0, 3.4 Hz, 1H), 1.00-0.89 (m, 2H), 0.78-0.70 (m, 2H). B: ¹H NMR (400 MHz, Chloroform-d) δ 9.93 (s, 1H), 7.41-7.17 (m, 5H), 6.42 (d, J=0.8 Hz, 1H), 5.50 (s, 2H), 1.69-1.58 (m, 1H), 0.93 (d, J=6.5 Hz, 2H), 0.69-0.59 (m, 2H).

### (ii) Synthesis of 1-benzyl-3-cyclopropyl-4-iodo-1H-pyrazole-5-carbaldehyde

[chemical structure]

Compound was prepared according to general procedure for iodination of pyrazole carbaldehyde from 200 mg of 1-benzyl-3-cyclopropyl-4-iodo-1H-pyrazole-5-carbaldehyde to give 284 mg of desired product. ¹H NMR (400 MHz, Chloroform-d) δ 9.71 (s, 1H), 7.36-7.18 (m, 5H), 5.60 (s, 2H), 1.86 (s, 1H), 1.04-0.90 (m, 4H).

### (iii) Synthesis of ethyl 1-benzyl-3-cyclopropyl-1,4-dihydropyrrolo[3,2-c]pyrazole-5-carboxylate

[chemical structure]

Compound was prepared according to general procedure for formation of pyrrolo[3,2-c]pyrazole cycle from 400 mg of 1-benzyl-3-cyclopropyl-4-iodo-1H-pyrazole-5-carbaldehyde to give 190 mg of desired product. LC-MS: M⁺ 310. ¹H NMR (400 MHz, Chloroform-d) δ 8.28 (s, 1H), 7.36-7.23 (m, 5H), 6.28 (d, J=1.7 Hz, 1H), 5.29 (s, 2H), 4.32 (q, J=7.1 Hz, 2H), 2.06 (tt, J=8.4, 5.1 Hz, 1H), 1.35 (t, J=7.1 Hz, 3H), 1.02-0.96 (m, 2H), 0.91 (ddd, J=4.9, 3.3, 2.5 Hz, 2H). ¹³C NMR (101 MHz, Chloroform-d) δ 162.09, 138.70, 136.63, 135.00, 128.66, 127.85, 127.83, 127.33, 116.23, 94.66, 60.88, 54.74, 14.40, 8.22, 6.50.

### (iv) Synthesis of ethyl 1-benzyl-3-cyclopropyl-6-iodo-1,4-dihydropyrrolo[3,2-c]pyrazole-5-carboxylate

[chemical structure]

Compound was prepared according to general procedure for iodination of pyrrolo[3,2-c]pyrazole from 123 mg of ethyl 1-benzyl-3-cyclopropyl-1,4-dihydropyrrolo[3,2-c]pyrazole-5-carboxylate to give 169 mg of desired product. LC-MS: M⁺ 436. ¹H NMR (400 MHz, Chloroform-d) δ 8.82 (s, 1H), 7.17 (dt, J=19.5, 7.6 Hz, 5H), 5.41 (s, 2H), 4.32 (q, J=7.1 Hz, 2H), 1.94 (s, 1H), 1.33 (t, J=7.1 Hz, 3H), 0.99-0.69 (m, 4H). ¹³C NMR (101 MHz, Chloroform-d) δ 161.27, 140.71, 137.89, 135.14, 128.77, 128.46, 127.44, 127.24, 126.75, 61.32, 52.19, 45.55, 14.36, 8.14, 6.67.

### (v) Synthesis of ethyl 1-benzyl-3-cyclopropyl-6-(4-fluorophenyl)-1,4-dihydropyrrolo[3,2-c]pyrazole-5-carboxylate

[chemical structure]

Compound was prepared according to general procedure for Suzuki coupling from 155 mg of ethyl 2-benzyl-3-cyclopropyl-6-iodo-2,4-dihydropyrrolo[3,2-c]pyrazole-5-carboxylate and 4-fluorophenylboronic acid and heated in microwave at 100° C. for 2 h to give 124 mg of desired product. LC-MS: M⁺ 404. ¹H NMR (400 MHz, Chloroform-d) δ 8.45 (s, 1H), 7.23-7.07 (m, 5H), 6.99 (t, J=8.6 Hz, 2H), 6.78-6.68 (m, 2H), 5.09 (s, 2H), 4.18 (q, J=7.1 Hz, 2H), 2.09 (s, 1H), 1.13 (t, J=7.1 Hz, 3H), 0.98 (ddt, J=25.7, 5.5, 2.2 Hz, 4H).

### (vi) Synthesis of ethyl 3-cyclopropyl-6-(4-fluorophenyl)-2,4-dihydropyrrolo[3,2-c]pyrazole-5-carboxylate

[chemical structure]

Compound was prepared according to general procedure for benzyl deprotection (catalytic hydrogenation) from 144 mg of ethyl 1-benzyl-3-cyclopropyl-6-(4-fluorophenyl)-1,4-dihydropyrrolo[3,2-c]pyrazole-5-carboxylate to give 110 mg of desired product. ¹H NMR (400 MHz, DMSO-d₆) δ 11.31 (s, 1H), 7.69-7.58 (m, 2H), 7.26-7.18 (m, 2H), 4.23 (qd, J=7.1, 1.9 Hz, 2H), 2.17-2.06 (m, 1H), 1.23 (td, J=7.1, 3.8 Hz, 3H), 0.97-0.88 (m, 4H).

### (vii) Synthesis of 3-cyclopropyl-6-(4-fluorophenyl)-1,4-dihydropyrrolo[3,2-c]pyrazole-5-carboxylic acid

[chemical structure]

Compound was prepared according to general procedure for ester hydrolysis from 50 mg of ethyl 3-cyclopropyl-6-(4-fluorophenyl)-1,4-dihydropyrrolo[3,2-c]pyrazole-5-carboxylate at 60° C. for 3 h to give 20 mg of desired product after recrystallisation from methanol/water. LC-MS: M⁺ 286; M⁻ 240, 284, 569. ¹H NMR (400 MHz, DMSO-d6) δ 12.78-11.59 (m, 2H), 11.21 (s, 1H), 7.66 (dd, J=8.8, 5.6 Hz, 2H), 7.21 (t, J=8.9 Hz, 2H), 2.11 (s, 1H), 0.97-0.73 (m, 4H). ¹³C NMR (101 MHz, DMSO) δ 163.67, 160.10, 131.88, 131.80, 130.26, 126.49, 125.05, 115.10, 114.89, 8.41, 7.70.

### Example 38—Preparation of 1-benzyl-3-cyclopropyl-1,4-dihydropyrrolo[3,2-c]pyrazole-5-carboxylic acid

### (i) Synthesis of 1-benzyl-3-cyclopropyl-1,4-dihydropyrrolo[3,2-c]pyrazole-5-carboxylic acid

[chemical structure]

Compound was prepared according to general procedure for ester hydrolysis from 18 mg of ethyl 1-benzyl-3-cyclopropyl-1,4-dihydropyrrolo[3,2-c]pyrazole-5-carboxylate at room temperature for 12 h to give 8 mg of desired product after prep HPLC. LC-MS: M⁺ 282; M⁻ 236, 280, 561. ¹H NMR (400 MHz, Chloroform-d) δ 8.39 (s, 1H), 7.30 (m, 4H), 6.36 (s, 1H), 5.31 (s, 2H), 2.07 (s, 1H), 1.00 (d, J=11.2 Hz, 2H), 0.96-0.84 (m, 2H).

### Example 39—Preparation of 3-cyclopropyl-6-(4-fluorophenyl)-1-methyl-1,4-dihydropyrrolo[3,2-c]pyrazole-5-carboxylic acid

### (i) Synthesis of ethyl 3-cyclopropyl-6-(4-fluorophenyl)-1-methyl-1,4-dihydropyrrolo[3,2-c]pyrazole-5-carboxylate (A) and ethyl 3-cyclopropyl-6-(4-fluorophenyl)-2-methyl-2,4-dihydropyrrolo[3,2-c]pyrazole-5-carboxylate (B)

[chemical structure]

Trimethylsilyl diazomethane (2 eq) was added dropwise to a vigorously stirred solution of ethyl 3-cyclopropyl-6-(4-fluorophenyl)-1,4-dihydropyrrolo[3,2-c]pyrazole-5-carboxylate (40 mg) in dry DCM (2 ml) and FBA (1 eq) at 0° C. during 20 min. The yellow colour of trimethylsilyl diazomethane immediately disappeared with evolution of nitrogen gas. Stirring at 0° C. was continued, and three further portions of trimethylsilyl diazomethane were added dropwise with intervals of 20 min. The reaction mixture was stirred for further 30 min, neutralized with triethylamine and concentrated. The residue was redissolved in ethylacetate and washed with H₂O. After drying over magnesium sulfate and concentration to dryness crude product was purified by column chromatography on silica (Biotage Isolera 4 g GraceRes column, EA/isohexane 5-100%). Two main fractions were obtained: 7 mg of A ¹H NMR (400 MHz, Chloroform-d) δ 8.40 (s, 1H), 7.47-7.39 (m, 2H), 7.15-7.06 (m, 2H), 4.22 (q, J=7.1 Hz, 2H), 3.63 (s, 3H), 2.04 (ddd, J=10.3, 7.7, 4.2 Hz, 1H), 1.18 (t, J=7.1 Hz, 3H), 1.08-0.96 (m, 2H), 0.95-0.87 (m, J=3.0 Hz, 2H). 15 mg of B: ¹H NMR (400 MHz, Chloroform-d) δ 7.98 (s, 1H), 7.89-7.69 (m, 2H), 7.08 (t, J=8.2 Hz, 2H), 4.32 (q, J=7.0 Hz, 2H), 4.11 (q, J=5.1 Hz, 2H), 1.98 (m, J=47.6 Hz, 4H), 1.30 (t, J=7.1 Hz, 3H), 1.27-1.23 (m, 1H), 1.11 (d, J=6.4 Hz, 2H), 0.88-0.77 (m, 2H).

### (ii) Synthesis of 3-cyclopropyl-6-(4-fluorophenyl)-1-methyl-1,4-dihydropyrrolo[3,2-c]pyrazole-5-carboxylic acid

[chemical structure]

Compound was prepared according to general procedure for ester hydrolysis from 7 mg of ethyl 3-cyclopropyl-6-(4-fluorophenyl)-1-methyl-1,4-dihydropyrrolo[3,2-c]pyrazole-5-carboxylate at room temperature for 16 h to give 4 mg of desired product after prep HPLC. LC-MS: M⁺ 300. 1H NMR (400 MHz, Methanol-d4) δ 7.38 (dd, J=8.8, 5.4 Hz, 2H), 7.04 (t, J=8.9 Hz, 2H), 3.47 (s, 3H), 2.03-1.92 (m, 1H), 0.93-0.79 (m, 4H).

### Example 40—Preparation of 3-cyclopropyl-6-(4-fluorophenyl)-2-methyl-2,4-dihydropyrrolo[3,2-c]pyrazole-5-carboxylic acid

### (i) Synthesis of 3-cyclopropyl-6-(4-fluorophenyl)-2-methyl-2,4-dihydropyrrolo[3,2-c]pyrazole-5-carboxylic acid

[chemical structure]

Compound was prepared according to general procedure for ester hydrolysis from 15 mg of ethyl 3-cyclopropyl-6-(4-fluorophenyl)-2-methyl-2,4-di hydropyrrolo[3,2-c]pyrazole-5-carboxylate at room temperature for 16 h to give 7.2 mg of desired product after prep HPLC. LC-MS: M⁺ 300. ¹H NMR (400 MHz, Methanol-d4): δ 7.59 (dd, J=8.7, 5.5 Hz, 2H), 7.01 (t, J=8.8 Hz, 2H), 4.01 (s, 3H), 2.02-1.92 (m, 1H), 1.13-1.06 (m, 2H), 0.93 (dd, J=4.9, 2.0 Hz, 2H).

### Example 41—Preparation of 2-benzyl-6-(3-chloro-4-((methylsulfonyl)methyl)phenyl)-3-cyclopropyl-2,4-dihydropyrrolo[3,2-c]pyrazole-5-carboxylic acid

### (i) Synthesis of 1-benzyl-5-cyclopropyl-4-iodo-1H-pyrazole-3-carbaldehyde

[chemical structure]

Compound was prepared according to general procedure for iodination of pyrazole carbaldehyde from 258 mg of 1-benzyl-5-cyclopropyl-1H-pyrazole-3-carbaldehyde to give 238 mg of desired product. ¹H NMR (400 MHz, Chloroform-d) δ 9.93 (s, 1H), 7.39-7.28 (m, 3H), 7.22-7.14 (m, 2H), 5.54 (s, 2H), 1.52-1.42 (m, 1H), 1.11-1.03 (m, 2H), 0.90-0.82 (m, 2H).

### (ii) Synthesis of ethyl 2-benzyl-3-cyclopropyl-2,4-dihydropyrrolo[3,2-c]pyrazole-5-carboxylate

[chemical structure]

Compound was prepared according to general procedure for formation of pyrrolo[3,2-c]pyrazole cycle from 255 mg of 1-benzyl-5-cyclopropyl-4-iodo-1H-pyrazole-3-carbaldehyde to give 105 mg of desired product. LC-MS: M⁺ 310. ¹H NMR (400 MHz, Chloroform-d) δ 7.94 (s, 1H), 7.26-7.14 (m, 3H), 7.12-7.05 (m, 2H), 6.81 (d, J=1.4 Hz, 1H), 5.49 (s, 2H), 4.30 (q, J=7.1 Hz, 2H), 1.76-1.62 (m, 1H), 1.31 (t, J=7.1 Hz, 3H), 0.90 (dd, J=8.4, 2.0 Hz, 2H), 0.65 (dd, J=5.1, 1.8 Hz, 2H). ¹³C NMR (101 MHz, Chloroform-d) δ 162.35, 147.34, 137.18, 133.00, 128.68, 127.63, 126.99, 126.56, 123.92, 99.84, 60.99, 54.83, 14.41, 6.22, 5.28.

### (iii) Synthesis of ethyl 2-benzyl-3-cyclopropyl-6-iodo-2,4-dihydropyrrolo[3,2-c]pyrazole-5-carboxylate

[chemical structure]

Compound was prepared according to general procedure for iodination of pyrrolo[3,2-c]pyrazole from 80 mg of ethyl 2-benzyl-3-cyclopropyl-2,4-di hydropyrrolo[3,2-c]pyrazole-5-carboxylate to give 97 mg of desired product. LC-MS: M⁺ 436, M⁻ 434. ¹H NMR (400 MHz, Chloroform-d) δ 8.35 (s, 1H), 7.35-7.21 (m, 3H), 7.20-7.08 (m, 2H), 5.58 (s, 2H), 4.41 (q, J=7.1 Hz, 2H), 1.67 (ddd, J=8.3, 5.1, 3.1 Hz, 1H), 1.42 (t, J=7.1 Hz, 3H), 1.02-0.87 (m, 2H), 0.75-0.62 (m, 2H). ¹³C NMR (101 MHz, Chloroform-d) δ 161.36, 150.27, 136.85, 131.87, 128.65, 127.67, 126.97, 125.10, 124.81, 61.40, 55.21, 53.33, 14.34, 6.26, 5.55.

### (iv) Synthesis of ethyl 2-benzyl-6-(3-chloro-4-((methylsulfonyl)methyl)phenyl)-3-cyclopropyl-2,4-dihydropyrrolo[3,2-c]pyrazole-5-carboxylate

[chemical structure]

Compound was prepared according to general procedure for Suzuki coupling from 97 mg of ethyl 2-benzyl-3-cyclopropyl-6-iodo-2,4-di hydropyrrolo[3,2-c]pyrazole-5-carboxylate to give 88 mg of desired product. ¹H NMR (400 MHz, Chloroform-d) δ 8.16 (s, 1H), 8.03 (d, J=1.7 Hz, 1H), 7.91 (dd, J=8.1, 1.8 Hz, 1H), 7.60 (d, J=8.1 Hz, 1H), 7.36-7.23 (m, 4H), 7.20-7.11 (m, 2H), 5.60 (s, 2H), 4.49 (s, 2H), 4.35 (q, J=7.1 Hz, 2H), 4.12 (q, J=7.1 Hz, 1H), 2.82 (s, 3H), 2.77 (s, 1H), 2.05 (s, 1H), 1.75 (tt, J=8.2, 5.2 Hz, 1H), 1.33 (t, J=7.1 Hz, 3H), 1.29-1.21 (m, 3H), 1.07-0.96 (m, 2H), 0.80-0.68 (m, 2H). ¹³C NMR (101 MHz, Chloroform-d) δ 161.89, 147.17, 136.91, 135.82, 133.59, 132.29, 131.23, 129.39, 128.70, 127.72, 127.60, 126.91, 124.90, 124.73, 124.53, 61.35, 57.90, 55.05, 39.53, 24.87, 14.15, 6.35, 5.27.

### (v) Synthesis of 2-benzyl-6-(3-chloro-4-((methylsulfonyl)methyl)phenyl)-3-cyclopropyl-2,4-dihydropyrrolo[3,2-c]pyrazole-5-carboxylic acid

[chemical structure]

Compound was prepared according to general procedure for ester hydrolysis from 8 mg of ethyl 2-benzyl-6-(3-chloro-4-((methylsulfonyl)methyl)phenyl)-3-cyclopropyl-2,4-dihydro pyrrolo[3,2-c]pyrazole-5-carboxylate at 60° C. for 3 h to give 4 mg of desired product after prep HPLC. LC-MS: M⁺ 484, 525. ¹H NMR (400 MHz, DMSO-d6) δ 11.40 (s, 1H), 7.52 (dd, J=8.0, 1.2 Hz, 1H), 7.13 (dd, J=7.3, 1.2 Hz, 1H), 7.08-7.01 (m, 1H), 4.20 (s, 2H), 3.71 (p, J=6.8 Hz, 1H), 3.23 (s, 4H), 2.95 (s, 3H), 2.89 (d, J=5.8 Hz, 4H), 1.24 (d, J=6.8 Hz, 6H). ¹³C NMR (101 MHz, DMSO) δ 163.97, 134.35, 133.61, 129.38, 127.67, 120.63, 120.18, 117.59, 112.64, 50.67, 50.60, 44.79, 34.70, 27.34, 23.59.

### Example 42—Preparation of 6-(3-chloro-4-((methylsulfonyl)methyl)phenyl)-3-cyclopropyl-1,4-dimethyl-1,4-dihydropyrrolo[3,2-c]pyrazole-5-carboxylic acid

### (i) Synthesis of ethyl 6-(3-chloro-4-((methylsulfonyl)methyl)phenyl)-3-cyclopropyl-2,4-dihydropyrrolo[3,2-c]pyrazole-5-carboxylate

[chemical structure]

Compound was prepared according to general procedure for benzyl deprotection (catalytic hydrogenation) from 80 mg of ethyl 2-benzyl-6-(3-chloro-4-((methylsulfonyl)methyl)phenyl)-3-cyclopropyl-2,4-dihydropyrrolo[3,2-c]pyrazole-5-carboxylate to give 35 mg of desired product together with 5 mg of de-chlorinated product (ethyl 3-cyclopropyl-6-(4-((methylsulfonyl)methyl)phenyl)-2,4-dihydropyrrolo[3,2-c]pyrazole-5-carboxylate). ¹H NMR (400 MHz, Chloroform-d) δ 7.60 (d, J=7.9 Hz, 1H), 7.52 (d, J=1.7 Hz, 1H), 7.37 (dd, J=7.9, 1.8 Hz, 1H), 4.52 (s, 2H), 4.19-4.09 (m, 5H), 3.58 (s, 3H), 2.86 (s, 3H), 2.09-2.01 (m, 1H), 1.05 (t, J=7.1 Hz, 3H), 1.00-0.91 (m, 4H).

### (ii) Synthesis of ethyl 6-(3-chloro-4-((methylsulfonyl)methyl)phenyl)-3-cyclopropyl-1,4-dimethyl-1,4-dihydropyrrolo[3,2-c]pyrazole-5-carboxylate

[chemical structure]

Compound was prepared according to general procedure A for methylation of pyrrolo[3,2-c]pyrazole from 35 mg of ethyl 6-(3-chloro-4-((methylsulfonyl)methyl)phenyl)-3-cyclopropyl-1,4-dihydropyrrolo[3,2-c]pyrazole-5-carboxylate to give 5.2 mg of demethylated product (together with 12 mg of monomethylated product). ¹H NMR (400 MHz, Chloroform-d) δ 7.60 (d, J=7.9 Hz, 1H), 7.52 (d, J=1.7 Hz, 1H), 7.37 (dd, J=7.9, 1.8 Hz, 1H), 4.52 (s, 2H), 4.19-4.09 (m, 5H), 3.58 (s, 3H), 2.86 (s, 3H), 2.09-2.01 (m, 1H), 1.05 (t, J=7.1 Hz, 3H), 1.00-0.91 (m, 4H).

### (iii) Synthesis of 6-(3-chloro-4-((methylsulfonyl)methyl)phenyl)-3-cyclopropyl-1,4-dimethyl-1,4-dihydropyrrolo[3,2-c]pyrazole-5-carboxylic acid

[chemical structure]

Compound was prepared according to general procedure for ester hydrolysis from 6 mg of ethyl 6-(3-chloro-4-((methylsulfonyl)methyl)phenyl)-3-cyclopropyl-1,4-dimethyl-1,4-dihydro pyrrolo[3,2-c]pyrazole-5-carboxylate at 75° C. for 2 h to give 3.5 mg of desired product after prep HPLC. LC-MS: M⁺ 422. ¹H NMR (400 MHz, DMSO-d6) δ 7.59-7.53 (m, 2H), 7.44 (dd, J=7.9, 1.8 Hz, 1H), 4.69 (s, 2H), 4.05 (s, 3H), 3.04 (s, 3H), 2.14 (tt, J=8.3, 5.0 Hz, 1H), 0.95-0.88 (m, 2H), 0.86-0.79 (m, 2H). ¹³C NMR (101 MHz, DMSO) δ 162.89, 135.60, 135.56, 133.42, 132.84, 132.34, 131.32, 130.29, 129.37, 126.71, 125.18, 108.71, 56.49, 40.41, 37.12, 34.42, 7.02, 6.67.

### Example 43—Preparation of 3-cyclopropyl-6-(4-((methylsulfonyl)methyl)phenyl)-2,4-dihydropyrrolo[3,2-c]pyrazole-5-carboxylic acid

### (i) Synthesis of ethyl 3-cyclopropyl-6-(4-((methylsulfonyl)methyl)phenyl)-2,4-dihydropyrrolo[3,2-c]pyrazole-5-carboxylate

[chemical structure]

5 mg of the compound was obtained as byproduct in catalytic hydrogenation of ethyl 2-benzyl-6-(3-chloro-4-((methylsulfonyl)methyl)phenyl)-3-cyclopropyl-2,4-dihydropyrrolo[3,2-c]pyrazole-5-carboxylate. ¹H NMR (400 MHz, DMSO-d₆) δ 11.35 (s, 1H), 7.63 (d, J=8.2 Hz, 2H), 7.42 (d, J=8.2 Hz, 2H), 4.52 (s, 2H), 4.23 (q, J=7.1 Hz, 2H), 2.93 (s, 3H), 2.12 (tt, J=7.7, 6.2 Hz, 1H), 1.23 (t, J=7.1 Hz, 4H), 0.94-0.87 (m, 4H).

### (ii) Synthesis of 3-cyclopropyl-6-(4-((methylsulfonyl)methyl)phenyl)-2,4-dihydropyrrolo[3,2-c]pyrazole-5-carboxylic acid

[chemical structure]

Compound was prepared according to general procedure for ester hydrolysis from 5 mg of ethyl 3-cyclopropyl-6-(4-((methylsulfonyl)methyl)phenyl)-2,4-dihydropyrrolo[3,2-c]pyrazole-5-carboxylate at 75° C. for 2 h to give 2 mg of desired product after prep HPLC. LC-MS: M⁺ 280, 360, 401. ¹H NMR (400 MHz, Chloroform-d) δ 7.95 (s, 1H), 6.84 (d, J=1.4 Hz, 1H), 4.37 (qd, J=7.1, 0.7 Hz, 2H), 3.99 (d, J=0.9 Hz, 3H), 3.19 (p, J=6.9 Hz, 1H), 1.46-1.34 (m, 9H). ¹³C NMR (101 MHz, CDCl3) δ 162.43, 147.17, 132.91, 128.34, 125.67, 99.25, 60.97, 37.91, 25.70, 21.90, 14.41.

### Biological Activity

The biological activity of the compounds of the present invention was tested using standard assay protocols.⁷ The following representative enzymes NDM-1 (New Delhi metallo-β-lactamase-1), IMP-1 (Imipenemase-1), VIM-1 ((Veronese metallo-β-lactamase-1) and VIM-2 (Veronese metallo-β-lactamase-2) were selected from different clinically relevant B1 metallo-β-lactamases.

TABLE 1
pIC₅₀ values of MBLi and derivatives against VIM-2, IMP-1, NDM-1 and VIM-1

| | | pIC₅₀ | | | |
|---|---|---|---|---|---|
| Example | Compound Structure | VIM-2 | IMP-1 | NDM-1 | VIM-1 |
| 1 | [chemical structure] | 7.4 | 6.0 | 7.2 | 6.2 |
| 2 | [chemical structure] | 8.3 | 7.1 | 7.8* | 6.9 |
| 3 | [chemical structure] | 4.8 | <3.6 | 3.9 | <3.6 |
| 4 | [chemical structure] | 6.5 | 5.6 | 6.7 | 5.3 |
| 5 | [chemical structure] | 5.0 | 5.6 | 4.9 | <4.4 |
| 6 | [chemical structure] | 6.9 | 6.3 | 7.4 | 6.2 |
| 7 | [chemical structure] | 4.7 | <4.2 | 4.4 | <3.6 |
| 8 | [chemical structure] | 4.4 | <3.6 | 3.9 | <3.6 |
| 9 | [chemical structure] | 7.3 | 4.9 | 6.5 | 5.8 |
| 12 | [chemical structure] | 7.4 | 7.3 | 7.3 | 6.2 |
| 13 | [chemical structure] | 7.1 | 6.4 | 7.3 | 5.8 |
| 14 | [chemical structure] | 6.8 | 5.9 | 7.2 | 5.5 |

### In-Vitro Cell Based Work

Meropenem MICs were determined using the CLSI broth or agar microdilution protocol (Ref: Clinical and Laboratory Standards Institute. 2012. Methods for dilution antimicrobial susceptibility tests for bacteria that grow aerobically; 9th ed. Approved standard M07-A9. CLSI, Wayne, Pa.) in the absence of each inhibitor or in its presence at the concentration stated. In each case DMSO was used to dissolve inhibitors. The MIC is defined as the concentration of meropenem required to totally inhibit growth, as evidenced by an absence of optical density at 600 nm measured spectrophotometrically or by eye (Spectra Max 190; Molecular Devices, Wokingham, United Kingdom).

TABLE 2
Minimum inhibitory concentrations (MICs) of meropenem
for various strains of bacterial species.

| Strain | | Strain | MER MIC |
|---|---|---|---|
| Entry | Species | Comment | (mg/L) |
| 1 | Klebsiella pneumoniae | IMP-4 | 4 |
| 2 | E. coli | IMP-4 | 4 |
| 3 | E. coli | VIM-1 | 0.25 |
| 4 | Klebsiella pneumoniae | VIM-1 | 4 |
| 5 | Pseudomonas aeruginosa | VIM-2 | 32 |
| 6 | Klebsiella pneumoniae | NDM-1 | >128 |
| 7 | Klebsiella pneumoniae | NDM-1 + DHA + | 16 |
| | | CTX − M-15 | |

TABLE 3
Changes in the MIC of meropenem (log2 difference) in the presence
of 2 mg/L of various inhibitor compounds of the present invention.

| Strain | | Change in MIC value | | | | | |
|---|---|---|---|---|---|---|---|
| entry | Species | Example 18 | Example 13 | Example 14 | Example 12 | Example 2 | Example 6 |
| 1 | Klebsiella | 0 | −2 | −1 | −2 | −2 | −2 |
| | pneumoniae | | | | | | |
| 2 | E. coli | 0 | −2 | −1 | −2 | −2 | −2 |
| 3 | E. coli | −1 | −1 | −1 | −1 | −1 | −1 |
| 4 | Klebsiella | −3 | −2 | −1 | −2 | −2 | −1 |
| | pneumoniae | | | | | | |
| 5 | Pseudomonas | 0 | 0 | 0 | 0 | 0 | 0 |
| | aeruginosa | | | | | | |
| 6 | Klebsiella | 0 | −1 | −1 | −1 | −1 | −1 |
| | pneumoniae | | | | | | |
| 7 | Klebsiella | −2 | −2 | −2 | −1 | −1 | −1 |
| | pneumoniae | | | | | | |

TABLE 4
Changes in the MIC of meropenem (log2 difference) in the presence
of 4 mg/L of various inhibitor compounds of the present invention.

| Strain | | Change in MIC value | | | | | |
|---|---|---|---|---|---|---|---|
| entry | Species | Example 18 | Example 13 | Example 14 | Example 12 | Example 2 | Example 6 |
| 1 | Klebsiella | 0 | −2 | −1 | −2 | −2 | −2 |
| | pneumoniae | | | | | | |
| 2 | E. coli | 0 | −2 | −1 | −2 | −2 | −2 |
| 3 | E. coli | −1 | −1 | −1 | −1 | −1 | 0 |
| 4 | Klebsiella | −2 | −2 | −1 | −2 | −2 | −1 |
| | pneumoniae | | | | | | |
| 5 | Pseudomonas | 0 | 0 | 0 | 0 | 0 | 0 |
| | aeruginosa | | | | | | |
| 6 | Klebsiella | 0 | −1 | −1 | −1 | −1 | −1 |
| | pneumoniae | | | | | | |
| 7 | Klebsiella | −2 | −2 | −2 | −2 | −2 | −1 |
| | pneumoniae | | | | | | |

TABLE 5
Changes in the MIC of meropenem (log2 difference) in the presence
of 8 mg/L of various inhibitor compounds of the present invention.

| Strain | | Change in MIC value | | | | | |
|---|---|---|---|---|---|---|---|
| entry | Species | Example 18 | Example 13 | Example 14 | Example 12 | Example 2 | Example 6 |
| 1 | Klebsiella | 0 | −2 | −1 | −2 | −2 | −2 |
| | pneumoniae | | | | | | |
| 2 | E. coli | 0 | −2 | −1 | −3 | −3 | −2 |
| 3 | E. coli | −1 | −1 | −1 | −1 | −1 | −1 |
| 4 | Klebsiella | −3 | −2 | −1 | −2 | −2 | 0 |
| | pneumoniae | | | | | | |
| 5 | Pseudomonas | 0 | 0 | 0 | 0 | 0 | 0 |
| | aeruginosa | | | | | | |
| 6 | Klebsiella | 0 | −2 | −2 | −2 | −2 | −1 |
| | pneumoniae | | | | | | |
| 7 | Klebsiella | −2 | −2 | −3 | −3 | −3 | −1 |
| | pneumoniae | | | | | | |

While specific embodiments of the invention have been described for the purpose of reference and illustration, various modifications will be apparent to a person skilled in the art without departing from the scope of the invention as defined by the appended claims.

### REFERENCES

1. J. Antibiot., 2013, 66, 571-591.
2. Clin. Microbiol. Rev., 2005, 18, 306-325.
3. Antimicrob. Agents Chemother., 2010, 54, 969-976.
4. Expert Opin. Ther. Pat., 2013, 23, 1469-1481.
5. a) http://www.who.int/en/; b) https://www.gov.uk/; c) http://www.cdc.gov.
6. J. Hospit. Infect., 2015, 89, 241-247.
7. J Med Chem 56:6945-6953
8. Antimicrob. Agents Chemother., 2011, 55, 3635-3636 (http://aac.asm.org/content/55/7/3635.full.pdf)

1. A compound of formula I or II, or a pharmaceutically acceptable salt or solvate thereof, as shown below: [chemical structure]
wherein
A₁ is selected from C, N, O, S, S(O) or S(O)₂;
A₂ is selected from C, N, O, S, S(O) or S(O)₂;
A₃ is selected from C, N, O, S, S(O) or S(O)₂;
with the proviso that either:
(i) all of A₁, A₂ and A₃ are C;
(ii) one of A₁, A₂ and A₃ is selected from N, O, S, S(O) or S(O)₂ and the others are C; or
(iii) two of A₁, A₂ and A₃ are N and the other is C;
R₁ is selected from hydrogen, (1-4C)alkyl, (1-4C)alkylaryl or aryl, wherein each (1-4C)alkyl or aryl is optionally substituted by one or more substituent groups selected from oxo, halo, cyano, nitro, hydroxy, carboxy, NR^{1A}R^{1B} or (1-4C)alkoxy, wherein R^{1A} and R^{1B} are each independently selected from hydrogen or (1-2C)alkyl;
R₂ is selected from hydrogen or:
(i) C(O)OH;
(ii) —C(O)OR_{2A}, wherein R_{2A} is selected from (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
(iii) —C(O)NR_{2B}R_{2C}; wherein R_{2B} and R_{2C} are each independently selected from hydrogen, (1-6C)alkyl, (3-8C)cycloalkyl, (3-8C)cycloalkyl(1-2C)alkyl, aryl, aryl-(1-2C)alkyl, heteroaryl, heteroaryl-(1-2C)alkyl, heterocyclyl or heterocyclyl-(1-2C)alkyl, each of which is optionally substituted by one or more substituent groups R^{A};
(iv) —C(O)NR_{2D}NR_{2B}R_{2E}; wherein R_{2D} is selected from hydrogen or (1-6C)alkyl and R_{2B} and R_{2C} are as defined above;
(v) tetrazolyl;
(vi) triazolyl;
(vii) —B(OR_{2F})(OR_{2G}), wherein R_{2F} and R_{2G} are each independently selected from hydrogen, (1-6C)alkyl or R_{2F} and R_{2G} are linked such that, together with the B and O atoms, they form a 5 or 6-membered heterocyclic ring, which is optionally substituted by (1-2C)alkyl;
(viii) trifluoromethylketone;
(ix) cyano;
(x) —[C(O)]ᵥS(NR_{2X})(O)NR_{2B}R_{2C} (where v is 0 or 1), wherein R_{2X} is selected from hydrogen, (1-6C)alkyl, C(O)R_{X}, C(O)OR_{X}, aryl, aryl-(1-2C)alkyl, heteroaryl, heteroaryl-(1-2C)alkyl, heterocyclyl or heterocyclyl-(1-2C)alkyl, wherein R_{X} is selected from (1-6C)alkyl, aryl or heteroaryl, each of which is optionally substituted with one or more R_{A}, and wherein R_{2B} and R_{2C} are as defined above;
(xi) —[C(O)]_{w}N(R_{2B})S(NR_{2X})(O)R_{X} (where w is 0 or 1), wherein R_{2B}, R_{2X} and R_{X} are each as defined above; or
(xii) —S(O)_{y}NR_{2B}R_{2C} (wherein y is 1 or 2), and wherein R_{2B} and R_{2C} are as defined above,
and wherein R^{A} is selected from oxo, halo, cyano, nitro or a group of the formula:
—Y²—X²—Z²
wherein
Y² is absent or a linker group of the formula —[CR^{A1}R^{A2}]ₘ— in which m is an integer selected from 1, 2, 3 or 4, and R^{A1} and R^{A2} are each independently selected from hydrogen or (1-2C)alkyl;
X² is absent or —O—, —C(O)—, —C(O)O—, —OC(O)—, —CH(OR^{A3})—, —N(R^{A3})—, —N(R^{A3})—C(O)—, —N(R^{A3})—C(O)O—, —C(O)—N(R^{A3})—, —N(R^{A3})C(O)N(R^{A3})—, —S—, —SO—, —SO₂—, —S(O)₂N(R^{A3})—, or —N(R^{A3})SO₂— wherein R^{A3} is selected from hydrogen or methyl; and
Z² is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z² is optionally further substituted by one or more substituent groups independently selected from oxo, halo, cyano, nitro, hydroxy, carboxy, NR^{A4}R^{A5}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, heterocyclyl, heterocyclyloxy, heterocyclyl-(1-2C)alkyl, heteroaryl, heteroaryloxy, heteroaryl-(1-2C)alkyl, C(O)NR^{A4}R^{A5}, NR^{A4}C(O)R^{A5}, NR^{A4}S(O)₂R^{A5} and S(O)₂NR^{A4}R^{A5}; wherein R^{A4} and R^{A5} are each independently selected from hydrogen, (1-4C)alkyl or (3-6C)cycloalkyl or (3-6C)cycloalkyl(1-2C)alkyl; or R^{A4} and R^{A5} can be linked such that, together with the nitrogen atom to which they are attached, they form a 4-6 membered heterocyclic ring;
and wherein any alkyl, aryl, heterocyclyl or heteroaryl group present in a substituent group on Z² is optionally further substituted by halo, cyano, nitro, hydroxy, caboxy, NR^{A6}R^{A7}, (1-2C)alkoxy, or (1-2C)alkyl; wherein R^{A6} and R^{A7} are selected from hydrogen or (1-2C)alkyl;
R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
R^{B} is oxo, halo, cyano, nitro, hydroxy or a group:
—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl;
or R^{B3} and Z³ can be linked such that, together with the nitrogen atom to which they are attached, they form a 4-7 membered heterocyclic ring, which is optionally substituted by oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6};
m is 0 or 1;
R₄ is selected from halo, cyano, nitro, hydroxy or a group
—Y⁴—X⁴—Z⁴
wherein
Y⁴ is absent or a linker group of the formula —[CR^{4A}R⁴]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, —N(R^{4D})SO₂— or ═N—O— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring;
and wherein any alkyl, aryl, heterocyclyl or heteroaryl group present in a substituent group on Z⁴ is optionally further substituted by oxo, halo, cyano, nitro, hydroxy, carboxy, NR^{4G}R^{4H}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4G}, OC(O)R^{4H}, C(O)NR^{4G}R^{4H}, NR^{4G}C(O)R^{4H}, NR^{4G}S(O)₂R^{4H} and S(O)₂NR^{4G}R^{4H}; wherein R^{4G} and R^{4H} are each independently selected from hydrogen, (1-4C)alkyl or (3-6C)cycloalkyl or (3-6C)cycloalkyl(1-2C)alkyl; or R^{4G} and R^{4H} can be linked such that, together with the nitrogen atom to which they are attached, they form a 4-7 membered heterocyclic ring;
or R^{4C} and Z⁴ can be linked such that, together with the nitrogen atom to which they are attached, they form a 4-7 membered heterocyclic ring, which is optionally substituted by oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, or C(O)NR^{4E}R^{4F}, NR^{4E}C(O)R^{4F}, NR^{4E}S(O)₂R^{4F} and S(O)₂NR^{4E}R^{4F};
n is 0 or 1 and R₅ is selected from halo, cyano, nitro, hydroxy or a group
—Y⁵—X⁵—Z⁵
wherein:
Y⁵ is absent or a linker group of the formula —[CR^{5A}R^{5B}]_{q}— in which q is an integer selected from 1 or 2 and R^{5A} and R^{5B} are each independently selected from hydrogen or methyl;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, nitro, hydroxy, carboxy, NR^{5E}R^{5F} or (1-2C)alkoxy; wherein R^{5E} and R^{5F} are each independently selected from hydrogen or (1-2C)alkyl; or
n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, nitro, hydroxy or a group
—Y⁶—X⁶—Z⁶
wherein:
Y⁶ is absent or a linker group of the formula —[CR^{6A}R^{6B}]_{q}— in which q is an integer selected from 1 or 2 and R^{6A} and R^{6B} are each independently selected from hydrogen or methyl;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or (1-4C)alkyl.

2. A compound of formula I according to claim 1, or a pharmaceutically acceptable salt or solvate thereof, for use in the treatment of a bacterial infection in combination with a beta-lactam antibiotic.

3. A compound according to any one of claim 1 or 2, wherein R₁ is selected from hydrogen or (1-4C)alkyl which is optionally substituted by one or more substituent groups selected from oxo, halo, or (1-2C)alkoxy.

4. A compound according to any one of claims 1 to 3, wherein R₁ is hydrogen.

5. A compound according to any one of claims 1 to 4, wherein R₂ is selected from hydrogen or:
(i) —C(O)OH;
(ii) —C(O)NR_{2B}R_{2C}; wherein R_{2B} and R_{2C} are each independently selected from hydrogen, (1-6C)alkyl, (3-8C)cycloalkyl, aryl, heteroaryl or heterocyclyl, each of which is optionally substituted by one or more substituent groups R^{A};
(iii) —C(O)NR_{2D}NR_{2B}R_{2C}; wherein R_{2D} is selected from hydrogen or (1-2C)alkyl and R_{2B} and R_{2C} are as defined above;
(iv) tetrazolyl;
(v) —B(OR_{2F})(OR_{2G}), wherein R_{2F} and R_{2G} are each independently selected from hydrogen, (1-4C)alkyl or R_{2F} and R_{2G} are linked such that, together with the B and O atoms, they form a 5 or 6-membered heterocyclic ring, which is optionally substituted by (1-2C)alkyl;
and wherein R^{A} is selected from halo, cyano, or a group of the formula:
—X²—Z²
wherein
X² is absent or —C(O)—, —SO₂—; and
Z² is hydrogen, (1-6C)alkyl, aryl, or heteroaryl;
and wherein Z² is optionally further substituted by one or more substituent groups independently selected from halo, hydroxy, NR^{A4}R^{A5}, (1-4C)alkoxy or (1-4C)alkyl, wherein R^{A4} and R^{A5} are each independently selected from hydrogen, or (1-2C)alkyl.

6. A compound according to any one of claims 1 to 5, wherein R₂ is C(O)OH or tetrazolyl.

7. A compound according to any one of claims 1 to 6, wherein R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
R^{B} is halo, cyano, nitro, hydroxy or a group:
—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl; or R^{B5} and R^{B6} can be linked such that, together with the nitrogen atom to which they are attached, they form a 4-7 membered heterocyclic ring;
and wherein any alkyl, aryl, heterocyclyl or heteroaryl group present in a substituent group on Z³ is optionally further substituted by halo, cyano, nitro, hydroxy, caboxy, NR^{B7}R^{B8}, (1-2C)alkoxy, or (1-2C)alkyl; wherein R^{B7} and R^{B8} are selected from hydrogen or (1-2C)alkyl.

8. A compound according to any one of claims 1 to 7, wherein R₃ is selected from hydrogen, halo, cyano, hydroxyl, aryl, (1-6C)alkyl, (2-6C)alkenyl, (2-6C)alkynyl, (3-8C)cycloalkyl, (3-8C)cycloalkenyl, heteroaryl or heterocyclyl, wherein said aryl, (3-8C)cycloalkyl, (3-8C)cycloalkenyl, heteroaryl or heterocyclyl is optionally substituted by one or more R^{B};
R^{B} is halo, cyano, nitro, hydroxy or a group:
—Y³—X³—Z³
wherein
Y³ is absent or a linker group of the formula —[CR^{B1}R^{B2}]ₙ— in which n is an integer selected from 1, 2, 3 or 4, and R^{B1} and R^{B2} are each independently selected from hydrogen or (1-2C)alkyl;
X³ is absent or —O—, —C(O)—, —C(O)O—, —OC(O)—, —CH(OR^{B3})—, —N(R^{B3})—, —N(R^{B4})—C(O)—, —N(R^{B4})—C(O)O—, —C(O)—N(R^{B3})—, —N(R^{B4})C(O)N(R^{B3})—, —S—, —SO—, —SO₂—, —S(O)₂N(R^{B3})—, or —N(R^{B4})SO₂— wherein R^{B3} and R^{B4} are each independently selected from hydrogen or methyl; and
Z³ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z³ is optionally further substituted by one or more substituent groups independently selected from oxo, halo, cyano, nitro, hydroxy, carboxy, NR^{B5}R^{B6}, (1-4C)alkoxy, (1-4C)alkyl, (1-4C)alkanoyl, (1-4C)alkylsulphonyl, C(O)NR^{B5}R^{B6}, NR^{B5}C(O)R^{B6}, NR^{B5}S(O)₂R^{B6} and S(O)₂NR^{B5}R^{B6}; wherein R^{B5} and R^{B6} are each independently selected from hydrogen, (1-4C)alkyl or (3-6C)cycloalkyl or (3-6C)cycloalkyl(1-2C)alkyl.]

9. A compound according to any one of the preceding claims, wherein m is 0 or 1 and R₄ is selected from halo, cyano, nitro, hydroxy or a group
—Y⁴—X⁴—X⁴
wherein
Y⁴ is absent or a linker group of the formula —[CR^{4A}R^{4B}]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring;
and wherein any alkyl, aryl, heterocyclyl or heteroaryl group present in a substituent group on Z⁴ is optionally further substituted by halo, cyano, nitro, hydroxy, caboxy, NR^{4G}R^{4H}, (1-2C)alkoxy, or (1-2C)alkyl; wherein R^{4G} and R^{4H} are selected from hydrogen or (1-2C)alkyl.

10. A compound according to any one of the preceding claims, wherein m is 0 or 1 and R₄ is selected from halo, cyano, nitro, hydroxy or a group
—Y⁴—X⁴—Z⁴
wherein
Y⁴ is absent or a linker group of the formula —[CR^{4A}R^{4B}]ₚ— in which p is an integer selected from 1 or 2, 3 or 4, and R^{4A} and R^{4B} are each independently selected from hydrogen or (1-2C)alkyl;
X⁴ is absent or —O—, —C(O)—, —C(O)O—, —OC(O)—, —CH(OR^{4C})—, —N(R^{4C})—, —N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
Z⁴ is hydrogen, (1-6C)alkyl, (2-6C)alkenyl, (2-6C)alkynyl, aryl, (3-6C)cycloalkyl, (3-6C)cycloalkenyl, heteroaryl or heterocyclyl;
and wherein Z⁴ is optionally further substituted by one or more substituent groups independently selected from oxo, halo, cyano, nitro, hydroxy, carboxy, NR^{4E}R^{4F}, (1-4C)alkoxy, (1-4C)alkyl, (3-8C)cycloalkyl, (3-8C)cycloalkyl-(1-3C)alkyl, (1-4C)alkanoyl, (1-4C)alkylsulphonyl, aryl, aryloxy, aryl-(1-2C)alkyl, heterocyclyl, heterocyclyloxy, heterocyclyl-(1-2C)alkyl, heteroaryl, heteroaryloxy, heteroaryl-(1-2C)alkyl, Si[(1-4C)alkyl]₃, C(O)OR^{4E}, OC(O)R^{4E}, C(O)NR^{4E}R^{4F}, NR^{4B}C(O)R^{4F}, NR^{4E}S(O)₂R^{4F} and S(O)₂NR^{4E}R^{4F}; wherein R^{4E} and R^{4F} are each independently selected from hydrogen, (1-4C)alkyl or (3-6C)cycloalkyl or (3-6C)cycloalkyl(1-2C)alkyl; or R^{4E} and R^{4F} can be linked such that, together with the nitrogen atom to which they are attached, they form a 4-7 membered heterocyclic ring.

11. A compound according to any one of the preceding claims, wherein:
n is 0 or 1 and R₅ is selected from halo, cyano, hydroxy or a group
—Y⁵—X⁵—Z⁵
wherein:
Y⁵ is absent or a linker group of the formula —[CR^{5A}R^{5B}]_{q}— in which q is an integer selected from 1 or 2 and R^{5A} and R^{5B} are each independently selected from hydrogen or methyl;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, hydroxy, carboxy, NR^{5E}R^{5F} or (1-2C)alkoxy; wherein R^{5E} and R^{5F} are each independently selected from hydrogen or (1-2C)alkyl; or
n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, hydroxy or a group
—Y⁶—X⁶—Z⁶
wherein:
Y⁶ is absent or a linker group of the formula —[CR^{6A}R^{6B}]^{q}— in which q is an integer selected from 1 or 2 and R^{6A} and R^{6B} are each independently selected from hydrogen or methyl;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or (1-2C)alkyl.

12. A compound according to any one of the preceding claims, wherein:
n is 0 or 1 and R₅ is selected from halo, cyano, hydroxy or a group
—Y⁵—X⁵—Z⁵
wherein:
Y⁵ is absent;
X⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
Z⁵ is hydrogen or (1-4C)alkyl which is optionally further substituted by one or more substituent groups independently selected from oxo, halo, cyano, hydroxy, carboxy, NR^{5E}R^{5F} or (1-2C)alkoxy; wherein R^{5E} and R^{5F} are each independently selected from hydrogen or methyl; or
n is 2 and the R₅ groups are positioned on adjacent atoms and are linked such that, together with the atoms to which they are attached, they form a fused 4, 5, 6 or 7-membered ring carbocyclic or heterocyclic ring, a fused phenyl ring or a fused 5 or 6-membered heteroaromatic ring, each of which is optionally further substituted by one or more substituent groups independently selected from halo, cyano, hydroxy or a group
—Y⁶—X⁶—Z⁶
wherein:
Y⁶ is absent;
X⁶ is absent or —O—, —C(O)—, —C(O)O—, —OC(O)—, —CH(OR^{6C})—, —N(R^{6C})—, —N(R^{6D})—C(O)—, —N(R^{6D})—C(O)O—, —C(O)—N(R^{6C})—, —S—, —SO—, —SO₂—, —S(O)₂N(R^{6C})—, or —N(R^{6D})SO₂— wherein R^{6C} and R^{6D} are each independently selected from hydrogen or methyl; and
Z⁶ is hydrogen or (1-4C)alkyl which is optionally further substituted by one or more substituent groups independently selected from oxo, halo, cyano, nitro, hydroxy, carboxy, NR^{6E}R^{6F} or (1-2C)alkoxy; wherein R^{6E} and R^{6F} are each independently selected from hydrogen or methyl.

13. A compound according to any one of the preceding claims, wherein A₁, A₂ and A₃ are selected from one of the following options:
(i) A₁ is C, A₂ is C and A₃ is C;
(ii) A₁ is N, A₂ is C and A₃ is C;
(iii) A₁ is S, A₂ is C and A₃ is C;
(iv) A₁ is O, A₂ is C and A₃ is C;
(v) A₁ is C, A₂ is C and A₃ is S;
(vi) A₁ is C, A₂ is C and A₃ is S(O)₂;
(vii) A₁ is N, A₂ is N and A₃ is C;
(viii) A₁ is N, A₂ is C and A₃ is N.

14. A compound selected from any one of the following:
3-Bromo-6-(3,5-dichlorophenyl)-4H-thieno[3,2-b]pyrrole-5-carboxylic acid;
3-Cyclopropyl-6-(3,5-dichlorophenyl)-4H-thieno[3,2-b]pyrrole-5-carboxylic acid;
3-Bromo-4H-thieno[3,2-b]pyrrole-5-carboxylic acid;
3-Bromo-6-(4-fluorophenyl)-4H-thieno-[3,2-b]pyrrole-5-carboxylic acid;
3,6-Bis(4-fluorophenyl)-4H-thieno-[3,2-b]pyrrole-5-carboxylic acid;
3-Isopropenyl-6-(4-fluorophenyl)-4H-thieno-[3,2-b]pyrrole-5-carboxylic acid;
3-Isopropyl-4H-thieno[3,2-b]pyrrole-5-carboxylic acid;
3-Cyclopropyl-4H-thieno[3,2-b]pyrrole-5-carboxylic acid;
6-(3-Acetamidophenyl)-3-bromo-4H-thieno[3,2-b]pyrrole-5-carboxylic acid;
3-Isopropyl-6-(4-fluorophenyl)-4H-thieno-[3,2-b]pyrrole-5-carboxylic acid;
3-Cyclopropyl-6-(4-fluorophenyl)-4H-thieno-[3,2-b]pyrrole-5-carboxylic acid;
3-bromo-6-(4-sulfamoylphenyl)-4H-thieno-[3,2-b]pyrrole-5-carboxylic acid;
3-Cyano-6-(4-fluorophenyl)-4H-thieno-[3,2-b]pyrrole-5-carboxylic acid;
6-(4-fluorophenyl)-4H-thieno-[3,2-b]pyrrole-3,5-dicarboxylic acid;
4-(3,5-dichlorophenyl)-6H-thieno[2,3-b]pyrrole-5-carboxylic acid;
3-(1-Acetamidoethyl)-6-(3,5-dichlorophenyl)-4H-thieno[3,2-b]pyrrole-5-carboxylic acid;
6-(3-Chloro-4-((methylsulfonyl)methyl)phenyl)-3-isopropyl-4H-thieno[3,2-b]pyrrole-5-carboxylic acid;
3-Bromo-6-(3,5-dichlorophenyl)-5-(1H-tetrazol-5-yl)-4H-thieno[3,2-b]pyrrole;
6-(3,5-Dichlorophenyl)-3-isopropyl-5-(1H-tetrazol-5-yl)-4H-thieno[3,2-b]pyrrole;
6-(3,5-Dichlorophenyl)-3-methoxy-4H-thieno[3,2-b]pyrrole-5-carboxylic acid;
6-(3,5-Dichlorophenyl)-3-phenethoxy-4H-thieno[3,2-b]pyrrole-5-carboxylic acid;
3-acetyl-6-(3,5-Dichlorophenyl)-4H-thieno[3,2-b]pyrrole-5-carboxylic acid;
6-(3,5-Dichlorophenyl)-3-(1-hydroxyethyl)-4H-thieno[3,2-b]pyrrole-5-carboxylic acid;
6-(3,5-Dichlorophenyl)-3-(1-isopropoxyethyl)-4H-thieno[3,2-b]pyrrole-5-carboxylic acid;
3-(1-(Benzyloxy)ethyl)-6-(3,5-dichlorophenyl)-4H-thieno[3,2-b]pyrrole-5-carboxylic acid;
6-(3,5-Dichlorophenyl)-3-(1-(dimethylamino)ethyl)-4H-thieno[3,2-b]pyrrole-5-carboxylic acid;
3-(1-Acetamidovinyl)-6-(3,5-dichlorophenyl)-4H-thieno[3,2-b]pyrrole-5-carboxylic acid;
3-(1-Acetamidoethyl)-6-(3,5-dichlorophenyl)-4H-thieno[3,2-b]pyrrole-5-carboxylic acid;
6-(3,5-Dichlorophenyl)-3-(1-(methylsulfonamido)ethyl)-4H-thieno[3,2-b]pyrrole-5-carboxylic acid;
6-(3,5-Dichlorophenyl)-3-vinyl-4H-thieno[3,2-b]pyrrole-5-carboxylic acid;
6-(3,5-Dichlorophenyl)-3-(methylsulfonyl)-4H-thieno[3,2-b]pyrrole-5-carboxylic acid;
3-(Azetidin-3-ylsulfonyl)-6-(3,5-dichlorophenyl)-4H-thieno[3,2-b]pyrrole-5-carboxylic acid;
6-(4-Fluorophenyl)-3-(piperidin-4-yl)-4H-thieno[3,2-b]pyrrole-5-carboxylic acid;
6-(4-Fluorophenyl)-3-(1,2,3,6-tetrahydropyridin-4-yl)-4H-thieno[3,2-b]pyrrole-5-carboxylic acid;
3-(1-(tert-Butoxycarbonyl)-1,2,3,6-tetrahydropyridin-4-yl)-6-(4-fluorophenyl)-4H-thieno[3,2-b]pyrrole-5-carboxylic acid;
6-(4-Fluorophenyl)-3-(1H-tetrazol-5-yl)-4H-thieno[3,2-b]pyrrole-5-carboxylic acid;
3-Bromo-6-(3-((tert-butoxycarbonyl)amino)phenyl)-4H-thieno[3,2-b]pyrrole-5-carboxylic acid;
6-(3-Aminophenyl)-3-bromo-4H-thieno[3,2-b]pyrrole-5-carboxylic acid hydrochloride;
6-(1H-Benzo[d]imidazol-7-yl)-3-bromo-4H-thieno[3,2-b]pyrrole-5-carboxylic acid;
6-(1H-Benzo[d]imidazol-7-yl)-3-isopropyl-4H-thieno[3,2-b]pyrrole-5-carboxylic acid;
6-(2-Amino-1H-benzo[d]imidazol-7-yl)-3-bromo-4H-thieno[3,2-b]pyrrole-5-carboxylic acid;
6-(2-Amino-1H-benzo[d]imidazol-7-yl)-3-isopropyl-4H-thieno[3,2-b]pyrrole-5-carboxylic acid;
6-(2-Amino-1H-benzo[d]imidazol-7-yl)-3-(1-(methylsulfonamido)ethyl)-4H-thieno[3,2-b]pyrrole-5-carboxylic acid;
6-(3,5-Dichlorophenyl)-3-(piperidin-4-yloxy)-4H-thieno[3,2-b]pyrrole-5-carboxylic acid;
4-(3-Chloro-4-((methylsulfonyl)methyl)phenyl)-6H-thieno[2,3-b]pyrrole-5-carboxylic acid;
4-(3,5-Dichlorophenyl)-2-isopropyl-6H-thieno[2,3-b]pyrrole-5-carboxylic acid;
4-(4-(N,N-Dimethylsulfamoyl)phenyl)-2-isobutyl-6H-thieno[2,3-b]pyrrole-5-carboxylic acid;
4-(3,5-Dichlorophenyl)-6H-thieno[2,3-b]pyrrole-5-carboxylic acid 1,1-dioxide;
3-Bromo-4H-furo[3,2-b]pyrrole-5-carboxylic acid;
3-Bromo-6-(3,5-dichlorophenyl)-4H-furo[3,2-b]pyrrole-5-carboxylic acid;
3-Cyclopropyl-6-(3,5-dichlorophenyl)-4H-furo[3,2-b]pyrrole-5-carboxylic acid;
6-(3,5-Dichlorophenyl)-3-(prop-1-en-2-yl)-4H-furo[3,2-b]pyrrole-5-carboxylic acid;
6-(3,5-Dichlorophenyl)-3-isopropyl-4H-furo[3,2-b]pyrrole-5-carboxylic acid;
3-Cyclopropyl-1,4-dihydropyrrolo[3,2-c]pyrazole-5-carboxylic acid;
3-Cyclopropyl-1-((2-(trimethylsilyl)ethoxy)methyl)-1,4-dihydropyrrolo[3,2-c]pyrazole-5-carboxylic acid;
3-Cyclopropyl-6-(3,5-dichlorophenyl)-1-((2-(trimethylsilyl)ethoxy)methyl)-1,4-dihydropyrrolo[3,2-c]pyrazole-5-carboxylic acid;
3-Cyclopropyl-6-(3,5-dichlorophenyl)-1,4-dihydropyrrolo[3,2-c]pyrazole-5-carboxylic acid;
3-Cyclopropyl-1-(N,N-dimethylsulfamoyl)-1,4-dihydropyrrolo[3,2-c]pyrazole-5-carboxylic acid;
3-Cyclopropyl-6-(3,5-dichlorophenyl)-1-(N,N-dimethylsulfamoyl)-1,4-dihydropyrrolo[3,2-c]pyrazole-5-carboxylic acid;
6-(3-Chloro-4-((methylsulfonyl)methyl)phenyl)-3-cyclopropyl-1-(N,N-dimethylsulfamoyl)-1,4-dihydropyrrolo[3,2-c]pyrazole-5-carboxylic acid;
(E)-3-(piperidin-4-yl)-6-styryl-3,4-dihydropyrrolo[2,3-d]imidazole-5-carboxylic acid;
3-(2-chlorobenzyl)-5,8-dihydro-1H,6H-pyrano[3′,4′:4,5]thieno[3,2-b]pyrrole-2-carboxylic acid;
3-(3-chlorophenyl)-8-isopropyl-1,8-dihydropyrrolo[2,3-b]indole-2-carboxylic acid;
3-(3,5-dichlorophenyl)-4-methyl-1,4-dihydropyrrolo[3,2-b]indole-2-carboxylic acid;
7-(2-aminoethoxy)-3-(3,5-dichlorophenyl)-1H-benzofuro[3,2-b]pyrrole-2-carboxylic acid; or
6-(3,5-dichlorophenyl)-3-ethyl-2-methyl-4H-furo[3,2-b]pyrrole-5-carboxylic acid 3,6-bis(3-chloro-4-((methylsulfonyl)methyl)phenyl)-4H-thieno[3,2-b]pyrrole-5-carboxylic acid;
6-(3-chloro-4-((methylsulfonyl)methyl)phenyl)-3-cyclopropyl-4H-thieno[3,2-b]pyrrole-5-carboxylic acid;
(E)-6-(3-chloro-4-((methylsulfonyl)methyl)phenyl)-3-(1-((2-(dimethylamino)ethoxy)imino)ethyl)-4H-thieno[3,2-b]pyrrole-5-carboxylic acid;
6-(3-chloro-4-((methylsulfonyl)methyl)phenyl)-4H-thieno[3,2-b]pyrrole-5-carboxylic acid;
3-acetyl-6-(3,5-dichlorophenyl)-4H-thieno[3,2-b]pyrrole-5-carboxylic acid;
6-acetyl-3-(3,5-dichlorophenyl)-2-methyl-4H-thieno[3,2-b]pyrrole-5-carboxylic acid 6-acetyl-3-(3,5-dichlorophenyl)-4H-thieno[3,2-b]pyrrole-5-carboxylic acid;
6-(3,5-dichlorophenyl)-3-(1H-tetrazol-5-yl)-4H-thieno[3,2-b]pyrrole-5-carboxylic acid;
3-(1-(tert-butoxycarbonyl)-1,2,3,6-tetrahydropyridin-4-yl)-6-(3,5-dichlorophenyl)-4H-thieno[3,2-b]pyrrole-5-carboxylic acid;
3-(1-(tert-butoxycarbonyl)piperidin-4-yl)-6-(3,5-dichlorophenyl)-4H-thieno[3,2-b]pyrrole-5-carboxylic acid;
6-(3,5-dichlorophenyl)-3-(5-(hydroxymethyl)-1,2,4-oxadiazol-3-yl)-4H-thieno[3,2-b]pyrrole-5-carboxylic acid 6-(3,5-dichlorophenyl)-3-(1-(methylsulfonyl)piperidin-4-yl)-4H-thieno[3,2-b]pyrrole-5-carboxylic acid;
6-(3,5-dichlorophenyl)-3-(piperidin-4-yl)-4H-thieno[3,2-b]pyrrole;
2-benzyl-6-(3-chloro-4-((methylsulfonyl)methyl)phenyl)-3-isopropyl-1-methyl-1,2-dihydropyrrolo[3,2-c]pyrazole-5-carboxylic acid;
3-isopropyl-2-methyl-6-(4-((methylsulfonyl)methyl)phenyl)-2,4-dihydropyrrolo[3,2-c]pyrazole-5-carboxylic acid;
3-isopropyl-1-methyl-6-(4-((methylsulfonyl)methyl)phenyl)-1,4-dihydropyrrolo[3,2-c]pyrazole-5-carboxylic acid;
6-(3-chloro-4-((methylsulfonyl)methyl)phenyl)-3-isopropyl-1,2-dimethyl-1,2-dihydropyrrolo[3,2-c]pyrazole-5-carboxylic acid;
6-(3-chloro-4-((methylsulfonyl)methyl)phenyl)-3-isopropyl-1-methyl-1,4-dihydropyrrolo[3,2-c]pyrazole-5-carboxylic acid;
6-(3-chloro-4-((methylsulfonyl)methyl)phenyl)-3-isopropyl-2-methyl-2,4-dihydropyrrolo[3,2-c]pyrazole-5-carboxylic acid;
2-benzyl-6-(3-chloro-4-((methylsulfonyl)methyl)phenyl)-3-cyclopropyl-2,4-dihydropyrrolo[3,2-c]pyrazole-5-carboxylic acid;
3-cyclopropyl-6-(4-((methylsulfonyl)methyl)phenyl)-2,4-dihydropyrrolo[3,2-c]pyrazole-5-carboxylic acid;
6-(3-chloro-4-((methylsulfonyl)methyl)phenyl)-3-cyclopropyl-1,4-dimethyl-1,4-dihydropyrrolo[3,2-c]pyrazole-5-carboxylic acid;
1-benzyl-3-cyclopropyl-1,4-dihydropyrrolo[3,2-c]pyrazole-5-carboxylic acid;
3-cyclopropyl-6-(4-fluorophenyl)-1,4-dihydropyrrolo[3,2-c]pyrazole-5-carboxylic acid;
3-cyclopropyl-6-(4-fluorophenyl)-2-methyl-2,4-di hydropyrrolo[3,2-c]pyrazole-5-carboxylic acid; or
3-cyclopropyl-6-(4-fluorophenyl)-1-methyl-1,4-di hydropyrrolo[3,2-c]pyrazole-5-carboxylic acid.

15. A pharmaceutical composition comprising a compound according to claims 1 to 14, or a pharmaceutically acceptable salt or solvate thereof, in admixture with a pharmaceutically acceptable diluent or carrier.

16. A pharmaceutical composition according to claim 15, for use in the treatment of a bacterial infection in combination with a beta-lactam antibiotic.

17. A method of treating a bacterial infection in a patient in need of such treatment, said method comprising administering to said patient a therapeutically effective amount of a compound according to claims 1 or 3 to 14, or a pharmaceutically acceptable salt or solvate thereof, or a pharmaceutical composition as defined in claim 15, in combination with a beta-lactam antibiotic.
